SEQUENCE LISTING (38 sequences)

SEQ ID NO: 1 - DNA, 10 bp, Artificial sequence
  5' - 3' sequence of ATC
ttactaccta

SEQ ID NO: 2 - DNA, 10 bp, Artificial sequence
  5' - 3' sequence of A*TC
  misc_feature (3)..(3): n is 7-deaza-2'-deoxyadenosine
  misc_feature (6)..(6): n is 7-deaza-2'-deoxyadenosine
  misc_feature (9)..(9): n is 7-deaza-2'-deoxyadenosine
ttnctnccnt

SEQ ID NO: 3 - DNA, 10 bp, Artificial sequence
  5' - 3' sequence of A**TC
  misc_feature (3)..(3): n is 7-deaza-8-aza-2'deoxyadenosine
  misc_feature (6)..(6): n is 7-deaza-8-aza-2'deoxyadenosine
  misc_feature (9)..(9): n is 7-deaza-8-aza-2'deoxyadenosine
ttnctnccnt

SEQ ID NO: 4 - DNA, 38 bp, Artificial sequcence
  Generic sequence of the oligonucleotide I
  misc_feature (13)..(13): n is C9 spacer
caaatccgttcanaggtcggtgtgaacggatttgagtc

SEQ ID NO: 5 - DNA, 16 bp, Artificial sequence
  Generic sequence of the oligonucleotide II
  misc_feature (2)..(2): n is dU with organic moiety attached
  misc_feature (5)..(12): each n is independently of each other C, T, 7-deaza adenosine, 7-deaza-8-aza adenosine or 7-deaza-8-aza guanosine
  misc_feature (13)..(13): n is 7-deaza adenosine or 7-deaza-8-aza adenosine
cnctnnnnnnnnncct

SEQ ID NO: 6 - DNA, 24 bp, Artificial sequence
  Generic sequence of the oligonucleotide II'
  misc_feature (9)..(9): n is inosine
  misc_feature (10)..(10): n is an abasic site
  misc_feature (11)..(11): n is an abasic site
  misc_feature (12)..(12): n is inosine
  misc_feature (13)..(13): n is an abasic site
  misc_feature (14)..(14): n is an abasic site
  misc_feature (15)..(15): n is inosine
  misc_feature (16)..(16): n is inosine
taggaggtnnnnnnnnagaggact

SEQ ID NO: 7 - DNA, 35 bp, Artificial sequence
  Generic sequence of the oligonucleotide III
cctagacttgactgacctcaactacatggtctaca

SEQ ID NO: 8 - DNA, 31 bp, Artificial sequence
  Generic sequence of the olionucleotide III`
tgtagaccatgtagttgaggtcagtcaagtc

SEQ ID NO: 9 - DNA, 21 bp, Artificial sequence
  Forward primer
aggtcggtgtgaacggatttg

SEQ ID NO: 10 - DNA, 23 bp, Artificial sequence
  Reverse primer
tgacctcaactacatggtctaca

SEQ ID NO: 11 - DNA, 23 bp, Artificial sequence
  Complementary sequence to reverse primer
tgtagaccatgtagttgaggtca

SEQ ID NO: 12 - DNA, 16 bp, Artificial sequence
  Sequence of exemplary oligonucleotide II (code A)
  misc_feature (2)..(2): n is T
  misc_feature (9)..(9): n is 7-deaza adenosine
  misc_feature (10)..(10): n is 7-deaza adenosine
  misc_feature (13)..(13): n is 7-deaza adenosine
cnctctttnnctncct

SEQ ID NO: 13 - DNA, 16 bp, Artificial sequence
  Sequence of exemplary oligonucleotide II (code B)
  misc_feature (2)..(2): n is T
  misc_feature (9)..(9): n is 7-deaza-8-aza adenosine
  misc_feature (10)..(10): n is 7-deaza-8-aza adenosine
  misc_feature (13)..(13): n is 7-deaza-8-aza adenosine
cnctctttnnctncct

SEQ ID NO: 14 - DNA, 16 bp, Artificial sequence
  Sequence of exemplary oligonucleotide II (code C)
  misc_feature (2)..(2): n is T
  misc_feature (9)..(9): n is 7-deaza-8-aza guanosine
  misc_feature (10)..(10): n is 7-deaza-8-aza guanosine
  misc_feature (13)..(13): n is 7-deaza-8-aza adenosine
cnctctttnnctncct

SEQ ID NO: 15 - DNA, 54 bp, Artificial sequence
  Forward primer
tcgtcggcagcgtcagatgtgtataagagacagaggtcggtgtgaacggatttg

SEQ ID NO: 16 - DNA, 57 bp, Artificial sequence
  Reverse primer
gtctcgtgggctcggagatgtgtataagagacagtgtagaccatgtagttgaggtca

SEQ ID NO: 17 - DNA, 24 bp, Artificial sequence
  Sequence of the oligonucleotide II`
  misc_feature (9)..(9): n is inosine
  misc_feature (10)..(10): n is an abasic site
  misc_feature (11)..(11): n is an abasic site
  misc_feature (12)..(12): n is inosine
  misc_feature (13)..(13): n is inosine
  misc_feature (14)..(14): n is an abasic site
  misc_feature (15)..(15): n is an abasic site
  misc_feature (16)..(16): n is inosine
atacaggtnnnnnnnnagaggact

SEQ ID NO: 18 - DNA, 12 bp, Artificial sequence
  Sequence of the oligonucleotide III
  misc_feature (1)..(8): each n is independently of each other C, T, A or G
gtatcaagcagg

SEQ ID NO: 19 - DNA, 12 bp, Artificial sequence
  Sequence of the oligonucleotide III`
  misc_feature (5)..(12): each n is independently of each other C, T, A or G
taggcctgcttg

SEQ ID NO: 20 - DNA, 35 bp, Artificial sequence
  Sequence of the oligonucleotide IV
  misc_feature (5)..(12): each n is independently of each other C, T, A or G
cctactctcgtatgacctcaactacatggtctaca

SEQ ID NO: 21 - DNA, 31 bp, Artificial sequence
  Sequence of the oligonucleotide IV`
  misc_feature (1)..(8): each n is independently of each other C, T, A or G
tgtagaccatgtagttgaggtcatacgagag

SEQ ID NO: 22 - DNA, 23 bp, Artificial
  Forward primer
aggtcggtgtgaacggatttgag

SEQ ID NO: 23 - DNA, 22 bp, Artificial
  Reverse Primer
gtagaccatgtagttgaggtca

SEQ ID NO: 24 - DNA, 10 bp, Artificial
  5' - 3' sequence of ATGC
gtcatgatct

SEQ ID NO: 25 - DNA, 10 bp, Artificial
  5' - 3' sequence of TG*C
  misc_feature (3)..(3): n is 7-deaza-8-aza-2'-deoxyguanosine
  misc_feature (6)..(6): n is 7-deaza-8-aza-2'-deoxyguanosine
  misc_feature (9)..(9): n is 7-deaza-8-aza-2'-deoxyguanosine
ttnctnccnt

SEQ ID NO: 26 - DNA, 10 bp, Artificial
  5' - 3' sequence of A*TG*C
  misc_feature (1)..(1): n is 7-deaza-8-aza-2'-deoxyguanosine
  misc_feature (4)..(4): n is 7-deaza-2'-deoxyadenosine
  misc_feature (6)..(6): n is 7-deaza-8-aza-2'-deoxyguanosine
  misc_feature (7)..(7): n is 7-deaza-2'-deoxyadenosine
ntcntnntct

SEQ ID NO: 27 - DNA, 16 bp, Artificial
  5' - 3' sequence of A*TC
  misc_feature (9)..(10): n is 7-deaza-2'-deoxyadenosine
  misc_feature (13)..(13): n is 7-deaza-2'-deoxyadenosine
ctctctttnnctncct

SEQ ID NO: 28 - DNA, 16 bp, Artificial
  5' - 3' sequence of A*TG*C
  misc_feature (6)..(6): n is 7-deaza-8-aza-2'-deoxyguanosine
  misc_feature (7)..(7): n is 7-deaza-2'-deoxyadenosine
  misc_feature (11)..(11): n is 7-deaza-8-aza-2'-deoxyguanosine
  misc_feature (13)..(13): n is 7-deaza-8-aza-2'-deoxyadenosine
ctctcnnttcncncct

SEQ ID NO: 29 - DNA, 15 bp, Artificial
  template sequence
  misc_feature (9)..(10): n is 7-deaza-8-aza-2'-deoxyadenosine
  misc_feature (12)..(12): n is a, c, g, or t
  misc_feature (13)..(13): n is 7-deaza-8-aza-2'-deoxyadenosine
ctctctttnctncct

SEQ ID NO: 30 - DNA, 16 bp, Artificial
  Code sequence IIa
  misc_feature (7)..(7): n is 7-deaza-2'-deoxyadenosine
  misc_feature (11)..(11): n is 7-deaza-2'-deoxyadenosine
  misc_feature (13)..(13): n is 7-deaza-2'-deoxyadenosine
ctctctntctntncct

SEQ ID NO: 31 - DNA, 16 bp, Artificial
  Code sequence IIb
  misc_feature (7)..(8): n is 7-deaza-2'-deoxyadenosine
  misc_feature (13)..(13): n is 7-deaza-2'-deoxyadenosine
ctcttcnnttccncct

SEQ ID NO: 32 - DNA, 16 bp, Artificial
  Code sequence IIc
  misc_feature (5)..(5): n is 7-deaza-2'-deoxyadenosine
  misc_feature (7)..(7): n is 7-deaza-2'-deoxyadenosine
  misc_feature (12)..(13): n is 7-deaza-2'-deoxyadenosine
ctctncnctttnncct

SEQ ID NO: 33 - DNA, 16 bp, Artificial
  Code sequence IId
  misc_feature (6)..(7): n is 7-deaza-2'-deoxyguanosine
  misc_feature (10)..(10): n is 7-deaza-2'-deoxyguanosine
  misc_feature (12)..(13): n is 7-deaza-2'-deoxyguanosine
ctctcnnttncnncct

SEQ ID NO: 34 - DNA, 16 bp, Artificial
  Code sequence IIe
  misc_feature (5)..(5): n is 7-deaza-2'-deoxyadenosine
  misc_feature (9)..(9): n is 7-deaza-2'-deoxyadenosine
  misc_feature (13)..(13): n is 7-deaza-2'-deoxyadenosine
ctctncctncttncct

SEQ ID NO: 35 - DNA, 16 bp, Artificial
  Code sequence IIf
  misc_feature (7)..(7): n is 7-deaza-8-aza-2'-deoxyguanosine
  misc_feature (11)..(11): n is 7-deaza-8-aza-2'-deoxyguanosine
  misc_feature (13)..(13): n is 7-deaza-2'-deoxyadenosine
ctctctntctntncct

SEQ ID NO: 36 - DNA, 16 bp, Artificial
  Code sequence IIg
  misc_feature (6)..(6): n is 7-deaza-8-aza-2'-deoxyguanosine
  misc_feature (7)..(7): n is 7-deaza-2'-deoxyadenosine
  misc_feature (11)..(11): n is 7-deaza-8-aza-2'-deoxyguanosine
  misc_feature (13)..(13): n is 7-deaza-2'-deoxyadenosine
ctctcnnttcncncct

SEQ ID NO: 37 - DNA, 16 bp, Artificial
  Code sequence IIh
  misc_feature (6)..(6): n is 7-deaza-2'-deoxyadenosine
  misc_feature (7)..(7): n is 7-deaza-8-aza-2'-deoxyguanosine
  misc_feature (8)..(8): n is 7-deaza-2'-deoxyadenosine
  misc_feature (13)..(13): n is 7-deaza-2'-deoxyadenosine
ctctcnnntttcncct

SEQ ID NO: 38 - DNA, 16 bp, Artificial
  Code sequence IIi
  misc_feature (6)..(6): n is 7-deaza-8-aza-2'-deoxyguanosine
  misc_feature (7)..(7): n is 7-deaza-2'-deoxyadenosine
  misc_feature (8)..(8): n is 7-deaza-8-aza-2'-deoxyguanosine
  misc_feature (13)..(13): n is 7-deaza-2'-deoxyadenosine
ctctcnnncctcncct

(19) United States
(12) Patent Application Publication
(10) Pub. No.: US 2023/0257908 A1
(43) Pub. Date: Aug. 17, 2023

(54) MODIFIED DNA-ENCODED CHEMICAL LIBRARY AND METHODS RELATED THERETO

(71) Applicant: TECHNISCHE UNIVERSITAET DORTMUND, Dortmund (DE)

(72) Inventors: Andreas BRUNSCHWEIGER, Dortmund (DE); Mateja KLIKA SKOPIC, Dortmund (DE); Marco POTOWSKI, Bottrop (DE); Verena Barbara Katharina KUNIG, Dortmund (DE)

(21) Appl. No.: 17/913,458

(22) PCT Filed: Mar. 25, 2021
(86) PCT No.: PCT/EP2021/057690

§ 371 Date: Sep. 22, 2022

(30) Foreign Application Priority Data
Mar. 27, 2020 (EP) 20166145.1

(51) Int. Cl. C40B 40/06; C07H 19/052; C07H 19/14; C07H 19/167; C07H 21/04

(52) U.S. Cl. CPC C40B 40/06; C07H 19/14; C07H 19/052; C07H 19/167; C07H 21/04

(57) ABSTRACT

A compound library may include a plurality of conjugate molecules, said conjugates comprising a small organic molecule covalently coupled to a nucleic acid moiety. The nucleic acid moiety may include or consist of 7-deazapurines and/or 7-deaza-8-azapurines, and, optionally, modified pyrimidine nucleotides and/or unmodified pyrimidine nucleotides. Further, a library for screening compounds binding to a target molecule and methods of synthesizing said library is also disclosed.

### REFERENCE TO A SEQUENCE LISTING SUBMITTED VIA EFS-WEB

The content of the ASCII text file of the sequence listing named “P85424US_seq_ST25”, which is 15 kb in size was created on Mar. 27, 2020; the sequence listing is electronically submitted via EFS-Web herewith and is herein incorporated by reference in its entirety.

### CROSS-REFERENCE TO RELATED APPLICATION

The present application is a national stage entry according to 35 U.S.C. §371 of PCT Application No.: PCT/EP2021/057690 filed on Mar. 25, 2021; which claims priority to European patent application 20166145.1, filed on Mar. 27, 2020; all of which are incorporated herein by reference in their entirety and for all purposes.

### TECHNICAL FIELD

The present disclosure lies in the field of medicinal chemistry and chemical biology and relates to a compound library comprising a plurality of conjugate molecules, wherein said conjugates comprise a small organic molecule covalently coupled to a nucleic acid moiety, wherein the nucleic acid moiety comprises or consists of 7-deazapurines and/or 7-deaza-8-azapurines, and, optionally, modified and/or unmodified pyrimidine nucleotides. Further, the present disclosure relates to the use of said library for screening compounds binding to a target molecule and methods of synthesizing said library.

### BACKGROUND

DNA-encoded chemical libraries (DELs) represent a technology for drug discovery. DELs feature the display of individual small organic chemical moieties on DNA tags that serve as amplifiable identification barcodes. The DNA-tag allows the simultaneous screening of very large compound collections (up to billions of molecules), because the DNA-tag of the relevant hit compounds can be identified and quantified by PCR-amplification and sequencing. They combine an ultra-high throughput compound screening approach with unbiased affinity-based compound identification. The most common approach to DNA-encoded libraries is the combinatorial mix-and-split synthesis strategy that combines preparative organic synthesis and encoding steps performed in an iterative manner.

Library design is enabled by a combination of starting materials and synthesis methodology. However, three factors pose serious limitations upon design of encoded libraries:
the chemical lability of DNA, e.g. DNA integrity can be compromised by depurination under many reaction conditions required for small molecule synthesis (see lit:.M. Potowski, F. Losch, E. Wunnemann, J. K. Dahmen, S. Chines, A. Brunschweiger, Screening of metal ions and organocatalysts on solid support-coupled DNA oligonucleotides guides design of DNA-encoded reactions. Chem. Sci. 2019 10, 10481-10492.);
the need to perform reactions in aqueous co-solvents; and
the need to use reactions with fast kinetics due to high dilution of DNA-coupled substrates.

A prerequisite for the library synthesis is the compatibility of DNA with the synthesis methodology used for synthesis of the organic chemical moieties. The main DNA degradation reaction is caused by depurination, i.e. the cleavage of purine bases from the DNA oligomer. DNA can be depurinated under acidic conditions and upon incubation with Lewis acids under forcing reaction conditions such as elevated temperature. (M. Potowski, F. Losch, E. Wunnemann, J. K. Dahmen, S. Chines, A. Brunschweiger, Screening of metal ions and organocatalysts on solid support-coupled DNA oligonucleotides guides design of DNA-encoded reactions. Chem. Sci. 2019 10, 10481-10492.)

Consequently, any reaction methodology meeting the compatibility requirement is very limited. A current challenge for DEL synthesis research is the development of novel synthetic schemes that furnish in DNA-compatible manner DNA-conjugates of small and geometrically defined (rigid) scaffolds that serve as starting points for subsequent combinatorial library synthesis. The synthesis of such molecules would result in molecules having a drug-like structure. These drug-like structures include, but are not limited to heterocyclic structures.

To date, the use of DNA that was composed of the nucleobases thymine, cytidine and 7-deazaadenine has been shown for barcoding of one-bead-one-compound peptide libraries (M. C. Needels, D. G. Jones, E. H. Tate, G. L. Heinkel, L. M. Kochersberger, W. J. Dower, R. W. Barrett, M. A. Gallop, Proc. Natl. Acad. Sci. USA, 1993, 90, 10700-10704). This technology required elongation of the code by chemical phosphoramidate DNA synthesis. It was previously demonstrated that the use of 7-deaza-8-azapurines for PCR amplification of 7-deaza-8-azapurine-containing DNA template strands was not possible (E. Eremeeva, et al., The 5-chlorouracil:7-deazaadenine base pair as an alternative to the dT:dA base pair. Org. Biomol. Chem., 2017, 15, 168).

### SUMMARY

Surprisingly, the present inventors found a synthesis strategy that enables the synthesis of several (hetero)cyclic structures from different starting materials attached to a specific DNA sequence by various catalytic methods. DNA sequences that comprise 7-deazapurines and/or 7-deaza-8-azapurines as purine bases and, optionally, modified and/or unmodified pyrimidine nucleotides are tolerant to a broad spectrum of reaction conditions, such as the use of strong acids or high concentrations of transition metals as catalysts; whereas, as experimentally proven, DNA molecules comprising natural purines adenine and guanine are degraded under the same reaction conditions by depurination and subsequent strand fragmentation of the resulting abasic sites. (M. Potowski, F. Losch, E. Wunnemann, J. K. Dahmen, S. Chines, A. Brunschweiger, Screening of metal ions and organocatalysts on solid support-coupled DNA oligonucleotides guides design of DNA-encoded reactions. Chem. Sci. 2019 10, 10481-10492.) The possibility to use a broad spectrum of reaction conditions allows for the synthesis of several heterocyclic structures attached to DNA sequences. In turn, this possibility to apply a broader spectrum of chemical reaction conditions results in the synthesis of a broad range of drug-like small molecule structures.

DNA-barcoding of compounds may require two enzymatic steps, preferably 5′-terminal phosphorylation of a first code sequence with polynucleotide kinase and ligation with a ligase, such as T4 ligase, to a second or further code sequence. The readout of the codes by sequencing requires prior amplification of the DNA strand with a polymerase, such as Taq polymerase. These enzymatic steps must be compatible with the chemical composition of the DNA. Surprisingly, the present inventors found that DNA sequences that comprise or consist of 7-deazapurines and/or 7-deaza-8-azapurines, and, optionally, modified and/or unmodified pyrimidine nucleotides can be phosphorylated at the 5′-terminus of the DNA sequence by a polynucleotide kinase. Surprisingly, DNA sequences containing the aforementioned chemical modifications may also be ligated by a DNA ligase, such as T4 DNA ligase. Surprisingly, DNA sequences ligated from chemically modified DNA fragments containing the aforementioned modified purine nucleobases can be read with high fidelity by Taq polymerase as experimentally demonstrated by Sanger sequencing for the first time (see Examples).

An encoding scheme may enable initiation of DNA-encoded library synthesis with controlled pore glass-coupled DNA strands (M. Potowski, V. Kunig, F. Losch, A. Brunschweiger, Synthesis of DNA-coupled isoquinolones and pyrrolidines by solid phase ytterbium- and silver-mediated imine chemistry. Med. Chem. Commun. 2019, 10, 1082-1093).

Herein, the application of this barcoding strategy for the use of chemically modified DNA strands and for ligating a pool of chemically stabilized DNA sequences to a PCR primer and to a second code is shown for the first time. A pool of chemically stabilized DNA oligonucleotides of different sequences can be annealed to a DNA oligonucleotide consisting of terminal complementary partial sequences and a degenerate middle sequence comprising inosine and stable abasic sites (see FIGS. 2-4; Tables 3-8).

In a first aspect, a compound library may include a plurality of conjugate molecules, wherein said conjugates comprise a small organic molecule covalently coupled to a nucleic acid moiety, wherein the nucleic acid moiety comprises or consists of 7-deazapurines and/or 7-deaza-8-azapurines, and, optionally, modified and/or unmodified pyrimidine nucleotides.

In various embodiments, the 7-deazapurines and/or 7-deaza-8-azapurines are selected from 7-deaza-adenosine, 7-deaza-guanosine, 7-deaza-8-azaadenosine, 7-deaza-8-azaguanosine, or combinations thereof, for example 7-deaza-8-azaadenosine, 7-deaza-8-azaguanosine, or combinations thereof.

In various embodiments, the 7-deazapurines and/or 7-deaza-8-azapurines comprise 7-aza- or 7-deaza-8-aza-modified inosine, 7-aza- or 7-deaza-8-aza-modified N⁶-methyladenosine, 7-aza-or 7-deaza-8-aza-modified xanthosine, or combinations thereof

In various embodiments, the plurality of conjugate molecules in the compound library comprises at least ten (different) molecules, preferably at least 15, at least 20, at least 30, at least 40 or at least 50 different molecules, more preferably at least 60, at least 70, at least 80, at least 90 or at least 100 different molecules. The molecules differ in the small organic moiety and, since each small organic moiety is identified by a specific nucleic acid sequence, also in their nucleic acid moiety.

In various embodiments, the nucleic acid moiety consists of at least 2, preferably at least 3, at least 4, at least 5, at least 6, at least 7, at least 8, at least 10 or more nucleotides. The upper limit is typically about 150 nucleotides in size, preferably up to 120 or up to 100. In various embodiments the nucleic acid moiety may comprise 60 to 90 nucleotides, such as 70 to 90 or 75 to 85. The nucleic acid moiety may comprise a code sequence that is used to identify the small organic moiety and additionally functional sequences needed for its detection, for example primer binding sites and the like.

In various embodiments, the nucleic acid moiety of the conjugate molecules comprises a sequence element of 3 to 18 nucleotides in length on its 5′ or 3′ end, preferably on the 5′ end, that consists exclusively of 7-deazapurines and/or 7-deaza-8-azapurines, and optionally modified and/or unmodified pyrimidine nucleotides.

In various embodiments, each conjugate has the structure

$$\text{Org-SNS-} \left(\text{INS}\right) \text{-TNS}$$

wherein “Org” represents the small organic molecule,
“SNS” represents a first nucleic acid identifier sequence that consists of 7-deazapurines and/or 7-deaza-8-azapurines, and, optionally, modified and/or unmodified pyrimidine nucleotides, “INS” represents an optional second nucleic acid identifier sequence that consists of 7-deazapurines and/or 7-deaza-8-azapurines, and, optionally, modified and/or unmodified pyrimidine nucleotides, and “TNS” represents a terminal nucleic acid identifier sequence that comprises unmodified purine residues,
wherein each of SNS and TNS is 3 to 18, 4 to 15, 4 to 12 or 5 to 10 nucleotides in length and INS is 3 to 50, 3 to 40, 3 to 18, 4 to 15, 4 to 12 or 5 to 10 nucleotides in length.

In various embodiments, the nucleic acid moiety is RNA, DNA or a mixture thereof. In more preferred embodiments, the nucleic acid moiety is DNA, i.e. the sugar moiety is deoxyribose.

In various embodiments, the nucleic acid moiety and the organic molecule are linked by an amide bond.

In further embodiments, a compound library may include conjugates that differ from each other by comprising different nucleic acid moieties and/or different small organic molecules. In various embodiments, each of the different small organic molecules is identifiable by a unique nucleic acid moiety that differs, in its sequence, from those coupled to other small organic molecules. This unique part of each nucleic acid moiety is also referred to herein as “code sequence”. The remainder of the nucleic acid moiety that provides the desired functionality, such as amplification primer binding sites, ligation sites, etc. may be the same for all nucleic acid moiety of a given population of conjugates, as long as the code sequence is still unique for each small organic moiety.

In various embodiments, the conjugate molecules of the library further comprise a linker portion between the nucleic acid moiety and the organic moiety molecule, this linker portion may be an polyoxyalkylene group, for example polyethylene glycol (PEG), an alkylene group, such as a methylene, ethylene or propylene group, or any other suitable linker group.

In various embodiments, the organic molecule consists of 2 or more carbon atoms, such as 3, 4, 5 or more carbon atoms, preferably up to 50 carbon atoms, more preferably up to 30 carbon atoms. It may comprise heteroatoms, for example selected from O, N, and S. In various embodiments, it may be a cyclic molecule, for example a heterocyclic molecule comprising one or more heteroatoms, for example selected from N, O and S. A more detailed definition of organic molecule is provided below.

In various embodiments, the organic molecule has a molecular weight of at most 900 daltons, preferably at most 700 daltons, and more preferably at most 500 daltons.

In various embodiments, the modified or unmodified pyrimidine nucleotides are selected from the group consisting of thymidine, cytidine, uridine, 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, 1-methylpseudouridine, 3-methylcytidine, 5-methylcytidine, 5-methylaminomethyluridine, 5-methoxyaminomethyl-2-thiouridine, 5-methoxycarbonylmethyl-2-thiouridine, 5-methoxycarbonylmethyluridine, 5-methoxyuridine, uridine-5-oxyacetic acid-methylester, uridine-5-oxyacetic acid, pseudouridine, 2-thiocytidine, 5-methyl-2-thiouridine, 2-thiouridine, 4-thiouridine, 5-methyluridine, 2′-O-alkyluridine, 2′-O-alkylthymidine, 2′-O-alkylcytidine and 3-(3-amino-3-carboxy-propyl)uridine. Also encompassed are combinations thereof.

In a further aspect, a method is disclosed for synthesizing a compound library, wherein said library comprises a plurality of conjugate molecules, wherein said conjugates comprise a small organic molecule covalently coupled to a nucleic acid moiety, the synthesis of each conjugate molecule comprises: (1) reacting a first nucleic acid consisting of 7-deazapurines and/or 7-deaza-8-azapurines and, optionally, modified and/or unmodified pyrimidine nucleotides (also referred to as “first code sequence”) with an organic molecule under conditions that allow the conjugation of said molecules; optionally, (2) subjecting the conjugate obtained in step (1) to reaction conditions that allow modification of the small organic molecule (without affecting the nucleic acid moiety); and, optionally, (3) elongating the first nucleic acid moiety of the conjugate obtained in step (2) by adding a further nucleic acid sequence (also referred to as “second or further code/nucleic acid sequence”). In step (1) of said method, each organic molecule is conjugated to a unique nucleic acid molecule that allows its identification. To avoid that the nucleic acid moiety is affected by the reaction in step (2), the nucleic acid moiety in step (1) consists of 7-deazapurines and/or 7-deaza-8-azapurines, and, optionally, modified and/or unmodified pyrimidine nucleotides, i.e. does not contain unmodified purine bases. The nucleic acid sequence added in step (3) can comprise modified and/or unmodified purine and pyrimidine nucleotides, as the reaction in step (2) is already completed. In various embodiments, it is also possible to repeat steps (2) and (3) multiple times. This allows synthesis of more complex organic molecules. Depending on the type of subsequent reaction the conjugate is subjected to after the first step (3), this requires that the elongation in step (3) is carried out using a suitable nucleic acid sequence, e.g. a nucleic acid sequence that consists of 7-deazapurines and/or 7-deaza-8-azapurines, and, optionally, modified and/or unmodified pyrimidine nucleotides, and only after the final reaction step (2) is completed, the elongation may be carried out with unmodified purine and, optionally pyrimidine bases. The nucleic acid sequences referred to in this context are the code sequences that serve to identify the coupled organic moiety. It is however contemplated in various embodiments that in addition to adding a code sequence that is used for identification of the organic moiety present in the conjugate, additional nucleotide sequence elements are added that, for example, provide for detectability of the conjugate, such as primer binding sites and the like.

In various preferred embodiments, the method comprises steps (2) and (3).

In various embodiments, the further nucleic acid sequence added in the elongation step (3) serves as an identifier nucleic acid sequence for the specific reaction step (2) and the first nucleic acid sequence identifies the organic molecule of step (1).

In the methods, steps (2) and (3) may be repeated at least once, i.e. steps (2) and (3) are followed by at least another cycle of steps (2) and (3). In such embodiments, the further nucleic acid sequence added in the first elongation step (3) is a second nucleic acid sequence and the further nucleic acid sequence added in the second or further elongation step (3) is a third or further nucleic acid sequence, wherein the second, third and further nucleic acid sequences are different from the fist nucleic acid sequence and different from each other. In such embodiments, the further nucleic acid sequence added in the final elongation step (3) may comprise unmodified purine nucleotides. In such embodiments, the further nucleic acid sequence(s) added in any elongation step (3) that is followed by another round of steps (2) and (3), consist of 7-deazapurines and/or 7-deaza-8-azapurines and, optionally modified and/or unmodified pyrimidine nucleotides, i.e. do not comprise unmodified purines.

In various embodiments of the method, the conjugate molecule obtained in step (1) is further used in a reaction comprising a) a strong acid, preferably an acid having a pKₐ of less than 3; and/or b) the presence of a metal catalyst selected from the group consisting of zinc, copper, silver, gold, ruthenium, iron, osmium, cobalt, rhodium, iridium, nickel, palladium, platinum, ytterbium, and derivatives thereof, preferably zinc, copper, silver, gold, ruthenium, ytterbium and derivatives thereof. This reaction may serve the purpose to convert the small organic molecule, the core structure/scaffold, to the desired drug candidate molecule. Due to the specific nature of the nucleic acid identifier sequence used in the conjugate, these are able to withstand these reaction conditions without adversely influencing nucleic acid structural integrity. This is, in various embodiments, the reaction generally referred to in step (2) above.

In a further aspect, a compound library may be obtainable by the methods.

In another aspect, a compound library may be used for screening a compound capable of binding to a target molecule. The compound may be screened based on its affinity for the target molecule, depending on reaction conditions for binding.

In various embodiments, the target molecule is a protein.

The disclosure also encompasses such methods, i.e. methods for screening a compound for its binding to target molecule, the target molecule preferably being a protein, said method comprising the use of a compound library.

It is understood that all combinations of the above disclosed embodiments are also intended to fall within the scope of the present invention.

### BRIEF DESCRIPTION OF THE DRAWINGS

The non-limiting embodiments will be better understood with reference to the detailed description when considered in conjunction with the non-limiting examples and the accompanying drawings.

FIG. 1 shows the synthesis of target molecules on controlled pore glass(CPG, black dot)-coupled DNA barcodes (two or three-cycle library). The designation of the sequence code 1 may be found in Tables 3-8, and code 1 identifies building block R_{A1-x}; FG is Functional group, and CPG is Controlled pore glass; the cleavage occurs with aqueous ammonia.

FIG. 2 shows the enzymatic phosphorylation of chemically modified code 1 DNA sequences II and degenerate counter strand II′ using a polynucleotide kinase (PNK).

FIG. 3 illustrates a two-cycle encoding strategy for short single-stranded DNA conjugates. The designation of the sequences may be found in Tables 3-8.

FIG. 4 shows a hairpin (three cycle) encoding strategy for short single-stranded DNA conjugates. The designation of the sequences may be found in Tables 3-8.

FIG. 5 depicts nucleosides used for barcoding of compounds. Sequence II is composed of chemically modified nucleosides; sequence II′ is composed of natural nucleosides and a degenerate region composed of inosine and stable abasic building blocks.

FIG. 6 shows an embodiment of a design of a final DNA-tagged compound and a PCR amplicon thereof.

FIG. 7 shows exemplary bifunctional organic starting compounds

FIG. 8 shows the gel electrophoresis of the stepwise ligation (steps 1 and 2) of 5 different 7-deazaA-modified DNA barcodes as schematically presented in FIG. 4. Gel analysis of the ligation reaction of the hairpin DNA I with the chemically modified duplex DNA IIₐ₋ₑ/II′ and native duplex DNA III/III′. Lane 1-5: one-pot ligation of hairpin DNA Iwith DNA duplex IIₐ₋ ₑ/II′ and DNA duplex III/III′, lane 6: DNA hairpin I. 7-deaza adenosine was used.

FIG. 9 shows the gel electrophoresis of the ligation (step 3) of 5 different 7-deazaA-modified DNA barcodes as schematically presented in FIG. 4. Gel analysis of the ligation reaction of the chemically modified ligation product I-IIₐ₋ₑIII/II′-III′ with native duplex DNA IV/IV′. Lane 1-5: ligation of I-IIₐ₋ₑIII/II′-III′ with DNA duplex IV/IV′. 7-deaza adenosine was used.

FIG. 10 shows the gel electrophoresis of the stepwise ligation (steps 1 and 2) of 4 different 7-deazaA-and 7-deaza-8azaG-modified DNA barcodes as schematically presented in FIG. 4. Gel analysis of the ligation reaction of the hairpin DNAI with the chemically modified duplex DNA II_{f-i}/II′ and native duplex DNA III/III′. Lane 1: DNA hairpin I; lane 2-5: one-pot ligation of hairpin DNA I with DNA duplex II_{f-i}/II′ and DNA duplex III/III′. 7-deaza adenosine and 7-deaza-8-aza guanosine was used.

FIG. 11 shows the gel electrophoresis of the ligation (step 3) of 4 different 7-deazaA-and 7-deaza-8azaG-modified DNA barcodes as schematically presented in FIG. 4. Gel analysis of the ligation reaction of the chemically modified ligation product I-II_{f-i}III/II′-III′ with native duplex DNA IV/IV′. Lane 1-4: ligation of I-II_{f-i}III/II′-III′ with DNA duplex IV/IV′. 7-deaza adenosine and 7-deaza-8-aza guanosine was used.

FIG. 12 shows the gel electrophoresis of PCR amplification products from the ligation products that are presented in FIG. 9. PCR amplification of chemically modified DNA ligation product. Lane 1-5: PCR product of DNA ligation product containing DNA hairpin I, chemically modified DNA duplex IIₐ₋ₑ/II′, native DNA duplexes III/III′ and IV/IV′. 7-deaza adenosine was used.

FIG. 13 shows the gel electrophoresis of PCR amplification products from the ligation products that are presented in FIG. 11. PCR amplification of chemically modified DNA ligation product. Lane 1-4: PCR product of DNA ligation product containing DNA hairpin I, chemically modified DNA duplex II_{f-i}/II′, native DNA duplexes III/III′ and IV/IV′. 7-deaza adenosine and 7-deaza-8-aza guanosine was used.

FIG. 14 shows (A) the analysis of an enzymatic DNA amplification with Taq polymerase by real-time PCR for one 7-deazaA-modified DNA ligation product shown in FIG. 9 and a control ligation product assembled from native DNA oligonucleotides. Amplification curves (qPCR) using different concentrations of DNA sequences containing native or stabilized DNA barcodes II_{d}. (B) the comparison of the melting curves of the amplicons of a7-deazaA-modified DNA ligation product shown in FIG. 9 and a control ligation product assembled from native DNA oligonucleotides. Melting curves of the PCR products after qPCR of different concentrations of DNA sequences containing native or stabilized DNA barcodes II_{d,} (C) the standard concentration curve for the analysis of an enzymatic DNA amplification with Taq polymerase by real-time PCR for one 7-deazaA-modified DNA ligation product shown in FIG. 9 and a control ligation product assembled from native DNA oligonucleotides. Standard curve (qPCR) using different concentrations of DNA sequences containing native or stabilized DNA barcodes II_{d}.

FIG. 15 shows(A) the analysis of an enzymatic DNA amplification with Taq polymerase by real-time PCR for one 7-deazaA- and 7-deaza-8-azaG-modified DNA ligation product shown in FIG. 9 and a control ligation product assembled from native DNA oligonucleotides. Amplification curves (qPCR) using different concentrations of DNA sequences containing native or stabilized DNA barcodes II_{f}; (B) the comparison of the melting curves of the amplicons of a 7-deazaA- and 7-deaza-8-azaG-modified DNA ligation product shown in FIG. 9 and a control ligation product assembled from native DNA oligonucleotides. Melting curves of the PCR products after qPCR of different concentrations of DNA sequences containing native or stabilized DNA barcodes II_{f}, (C) the standard concentration curve for the analysis of an enzymatic DNA amplification with Taq polymerase by real-time PCR for one 7-deazaA- and 7-deaza-8-azaG-modified DNA ligation product shown in FIG. 9 and a control ligation product assembled from native DNA oligonucleotides. Standard curve (qPCR) using different concentrations of DNA sequences containing native or stabilized DNA barcodes II_{f}..

### DETAILED DESCRIPTION

The present inventors surprisingly found that 7-deazapurines and/or 7-deaza-8-azapurines are not degraded under conditions commonly used for the synthesis of heterocyclic chemical compounds (for example reactions involving the use of strong acids or metal catalysts, such as zinc, copper, silver, gold, ruthenium, iron, osmium, cobalt, rhodium, iridium, nickel, palladium, platinum, ytterbium, and derivatives thereof), which is contrasted to most purine nucleotides that are degraded under the same conditions. Based on these results, the present inventors concluded that chemical compounds being labeled with DNA can be reacted with a broad spectrum of chemical structures, including but not limited to those necessary for forming heterocycles, if the DNA-label consists of 7-deazapurines and/or 7-deaza-8-azapurines (for example instead of unmodified purine bases) and optionally modified and/or unmodified pyrimidine nucleotides. If the reaction comprises multiple steps, after each step the conjugate may be further modified by adding a nucleic acid sequence by elongation of the existing sequence that serves to identify the reaction steps already carried out and thus identify the organic molecule part of the conjugate at each step of the reaction process. Said nucleic acid sequence added between those steps is typically of the same type as the one used for the first labeling step, as it needs to withstand the reaction conditions. At a later stage of the reaction, for example after the organic moiety of the compound has been reacted to its final chemical structure, it is also possible to elongate the DNA-label with further nucleotides including additional purine and/or pyrimidine nucleotides, with these additional nucleotides including the naturally occurring non-modified nucleotides. Specifically, the nucleic acid moiety (DNA-label) may be elongated after each reaction step/cycle that changes the organic moiety with additional DNA barcodes that comprise or consist of pyrimidine nucleotides; 7-deazapurines and/or 7-deaza-8-azapurines; native DNA nucleotides, or combinations thereof by an enzymatic ligation step, with the selection of the nucleotides used being dependent on the following reaction steps, if any, to allow the synthesis of the conjugate molecule. In all these elongation steps, the DNA barcodes may be selected such that they identify the organic moiety after the reaction. The elongated DNA label thus allows determination of the starting organic moiety (before the reaction by virtue of the original DNA label sequence) and the organic moiety after it has been subjected to a specific type of reaction (by virtue of the elongated DNA label sequence, in particular the added sequence).

In various embodiments, the nucleic acid moiety can comprise, in addition to the nucleotide sequence stretches used for identification of the small organic moiety, additional nucleic acid sequences that are not used for identification purposes but rather to allow processing of the conjugate, such as immobilization, further conjugation to other nucleic acid molecules, amplification or detection. These nucleic acid sequences may be added in an elongation step as defined herein, with “elongation step” generally referring to any type of reaction that adds nucleotides to the first code sequence, i.e. the nucleotide sequence added to the small organic moiety used as a starting scaffold/educt for its identification. Typically, these are added after the reaction of the organic moiety is completed to avoid limitation as to their sequence.

Thus, in a first aspect, a compound library may include a plurality of conjugate molecules, wherein said conjugates comprise a small organic molecule covalently coupled to a nucleic acid moiety, wherein the nucleic acid moiety comprises or consists of 7-deazapurines and/or 7-deaza-8-azapurines, and, optionally, modified and/or unmodified pyrimidine nucleotides. Each conjugate molecule has a different organic molecule from the other conjugate molecules, i.e. the pairs of organic molecule and nucleic acid moiety are unique in that each specific organic molecule is identified by one specific nucleic acid moiety that differs in sequence from other nucleic acid moieties used for conjugation to other organic molecules. “Different organic molecules”, as used in this context, refers to the type of organic molecule and not the individual molecule. For example, all phenyl moieties are paired with one sequence-specific nucleic acid moiety that thus serves as an identifier for a phenyl group in general, while all pyrazole moieties are paired with another sequence-specific nucleic acid moiety that consequently serves as an identifier for a pyrazole group. The difference in the nucleic acid sequence is, for example, limited to the so-called code sequence which may comprise or consist of a first and a second and, optionally, further code sequence, while other parts of the nucleic acid moiety may be similar in that they provide for a functionality desired for all conjugates, such as the ability to allow their amplification, etc. However, in various embodiments, also these sequences used for functional purposes may differ between the different conjugates, for example, so that discrimination can already be achieved at the amplification stage.

Since the compound library is produced by different steps that involve reacting a starting nucleic acid-organic moiety conjugate with other organic groups to build the desired organic molecule and in the course of these steps additional nucleic acid sequences are attached to the nucleic acid sequence of the starting nucleic acid-organic moiety conjugate that serve to identify the respective variations of the starting organic moiety caused by the reactions, the conjugate molecules of the library comprise a nucleic acid moiety that consists of different nucleotide sequence parts, namely a starting nucleotide sequence that is exclusively made from 7-deazapurines and/or 7-deaza-8-azapurines, and, optionally, modified and/or unmodified pyrimidine nucleotides, i.e. which does not contain any unmodified purine residues, and later added parts that consist of nucleotide sequences. Only the last added sequence part may comprise unmodified purine nucleotides, as only after all reactions of the organic moiety have been completed, the final nucleic acid tag is no longer subjected to the necessary reaction conditions and thus no longer at risk of being depurinated.

That step-wise synthesis strategy of the organic group and the corresponding nucleic acid tag, imparts a very specific structure to the nucleic acid tag in that it comprises a 5′ or 3′ region that is devoid of any unmodified purine nucleotides, while the respective other end may comprise such unmodified purine nucleotides. The 5′ or 3′ region of the nucleic acid tag that does not comprise any unmodified purine nucleotides corresponds to the starting nucleotide sequence, and optionally further sequences added in intermediary reaction steps, while the other end comprising unmodified purine residues corresponds to the nucleic acid sequence added as a final identifier tag part after all reactions of the organic group have been completed. All conjugates of the library thus have the schematic structure:

$$\text{Org-SNS-} \left(\text{INS}\right) \text{-TNS}$$

wherein “Org” represents the final organic group after all modifying reactions have been carried out, “SNS” represents the starting nucleic acid, i.e. the first short identifier sequence, that is exclusively made from 7-deazapurines and/or 7-deaza-8-azapurines, and, optionally, modified and/or unmodified pyrimidine nucleotides, “INS” represents the optional intermediary nucleic acid sequence identifier that may be added if the organic group is subjected to more than one reaction and then serve to identify each modification of the organic group, with said optional intermediary nucleic acid sequence identifiers being typically also free of unmodified purine nucleotides, as these may be depurinated in the following reaction steps, and “TNS” representing the terminal nucleic acid identifier sequence that may also comprise unmodified purine residues, as this conjugate will then not be subjected to further reactions of the organic group. The respective nucleotide sequences are of a length that allows identification of the starting organic group and all subsequent steps it has been subjected to. In various embodiments, each of these nucleotide sequence stretches is 3 to 18, 4 to 15, 4 to 12 or 5 to 10 nucleotides in length. The lower limit of the nucleic acid sequence at this stage may be 3, 4 or 5 nucleotides. The upper limit is typically 12, 11, 10, 9 or 8, for example 8-12 nucleotides. Accordingly, the at least 3, typically 3 to 18, 4 to 15, 4 to 12 or 5 to 10 nucleotides, closest to the linkage to the organic moiety consist exclusively of 7-deazapurines and/or 7-deaza-8-azapurines, and, optionally, modified and/or unmodified pyrimidine nucleotides but do not comprise an unmodified purine nucleotide.

The term “compound”, as used herein, relates to a substance formed when two or more chemical elements are chemically bonded together. A typical type of chemical bond in the compounds is a covalent bond. However, atoms within the compound may also be bound to each other by ionic bonds, metallic bonds and/or coordinate covalent bonds. “Compounds” refer generally to molecules potentially capable of structural interactions with extracellular or cellular constituents through covalent or non-covalent interactions, such as, for example, through hydrogen bonds, ionic bonds, van der Waals attractions, or hydrophobic interactions. For example, compounds will most typically include molecules with moieties necessary for structural interaction with proteins, glycoproteins, and/or other macromolecules. “Compound library”, as used herein, refers to any collection of agents/compounds that includes a plurality of molecular structures. Compound libraries can include, for example, combinatorial chemical libraries, natural products libraries, and peptide and cyclized peptide libraries. Compound libraries contain compounds that comprise a small organic molecule covalently coupled to a nucleic acid moiety. Such libraries are known as DNA-encoded chemical libraries (DELs).

“Barcode” and “barcoding” and “coding” may be used interchangeably and are defined herein to mean the nucleic acid moiety attached to the organic molecule. The nucleic acid moiety acts as a barcode to unambiguously identify each of the conjugates in the respective binding assays. These nucleic acid sequences having barcoding properties are also referred to as “code sequences” herein.

“Plurality”, as used herein, is defined as two or more than two, in particular 2, 3, 4, 5, 6, 7, 8, 9, 10 or more, more preferably 20 or more, 50 or more, 100 or more, 500 or more, 1000 or more, 1500 or more, 3000 or more, 5000 or more, 10000 or more or 50000 or more. In various embodiments, the library comprises at least 10 (different) molecules, preferably at least 20, at least 30, at least 40 or at least 50 different molecules, more preferably at least 100 different molecules. The term “plurality” as used herein refers to the type of molecule, not the number of molecules. A plurality of conjugates thus means that a multitude of conjugate molecules is present that differ in their organic moiety and their corresponding nucleic acid moiety independent on whether each type of these molecules is present in multiple copies or only a single copy. Typically, a library as defined herein comprises a plurality of different conjugates, with each type of conjugate being represented by a high number of identical molecules.

The term “conjugate molecule”, as used herein, refers to a compound comprising two or more molecules (e.g., organic molecules or nucleic acid molecules) that are chemically linked. The two or more molecules are chemically linked using any suitable chemical bond (e.g., covalent bond). Suitable chemical bonds are well known in the art and include but are not limited to amide bonds, disulfide bonds, thioester bonds, and ester bonds. According to non-limiting embodiments, the conjugates comprise a small organic molecule covalently coupled to a nucleic acid moiety. In various embodiments, the nucleic acid moiety and the organic molecule are linked by an amide bond. The linkage may be direct or via a linker moiety, such as those described herein below.

The term “organic molecule” refers to carbon-based chemicals, including small organic molecules and macromolecules, as well as derivatives and analogues thereof. The organic molecules may display biological activity such as, but not limited to pharmaceutical, antibiotic, pesticidical, herbicidical, or fungicidical activity. Preferably, the organic molecule, in particular the final organic molecule after reaction of the conjugate, contains a heterocyclic structure. The term “heterocyclic,” as used herein, means an aromatic or non-aromatic saturated mono-, bi-, or tricyclic ring system having 2 to 14 ring carbon atoms, containing 1-8 ring heteroatom or heteroatom groups, for example chosen from N, NH, N-(CO)-C₁₋₆-alkyl, NC₁₋₆-alkyl, O, SO, SO₂, S, P, or PO₄⁻³ alone or in combination. Bi- and tricyclic heterocyclic groups are fused at 2 or 4 points or joined at one point via a bond or a heteroatom linker (O, S, NH, or N(C₁-C₆ alkyl). The mono-, bi-, or tricyclic heterocyclic group can be optionally substituted on the ring by replacing an available hydrogen on the ring by one or more substituents which may be the same or different, each being independently selected from any suitable group, including but not limited to C1-10 alkyl, alkoxy, alkenyl, alkenyloxy, alkynyl or alkynyloxy, 5- to 14-membered aryl, heteroaryl, or cycloaliphatic, halogen, nitro, cyano, hydroxy, amino, and carboxy. In a non-limiting embodiment, there are no adjacent oxygen and/or sulfur ring atoms present in the ring system. Alternatively, adjacent oxygen and/or sulfur ring atoms are present in the ring system. The nitrogen or sulfur atom of the heterocyclic ring system can be optionally oxidized to the corresponding N-oxide, S-oxide or S-dioxide. Non-limiting examples of suitable heterocyclic ring system include furanyl, imidazolyl, isoxazolyl, oxadiazolyl, oxazolyl, pyrazolyl, pyrrolyl, pyridyl, pyrimidyl, pyridazinyl, thiazolyl, triazolyl, tetrazolyl, thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, isoindolyl, acridinyl, or benzoisoxazolyl. Non-limiting examples of suitable heterocyclic rings include also aziridinyl, piperidinyl, pyrrolidinyl, piperazinyl, tetrahydropyranyl, tetrahydrofuranyl, tetrahydrothiophenyl, morpholinyl, thiomorpholinyl and the like. Also included within the scope of the term “heterocyclic” as it is used herein is a group in which the heterocyclic ring is fused at two points or j oined at one point via a bond or a heteroatom linker (O, S, NH, or N(C₁-C₆ alkyl), to one aromatic, or cycloalkyl ring, non-limiting examples include isoindoline-1,3-dione 1-methyl-2-phenyl-1H-pyrazole-3(2H)-one, indoline, pyridoindole and the like.

The “organic molecule”, as used herein, may also refer to molecules of different classes such as small molecules conforming or not conforming to Lipinski’s rule of five, (cyclic) peptides, proteins, nucleotides, lipids, sugars and derivatives thereof. In one embodiment, the organic molecule can be selected from the group comprising small molecules conforming or not conforming to Lipinski’s rule of five, (cyclic) peptides, proteins, nucleotides and mixtures thereof.

“Small” in the context of the term “organic molecule”, as used herein, relates to compounds that consist of 2 or more, such as 3, 4, 5 or more carbon atoms, preferably up to 50 carbon atoms, more preferably up to 30 carbon atoms. In various embodiments, the small organic molecule has a molecular weight of at most 1500 daltons, preferably at most 700 daltons and more preferably at most 500 daltons. It may comprise heteroatoms, for example selected from O, N, and S. In various embodiments, it may be a cyclic molecule, for example a heterocyclic molecule comprising one or more heteroatoms, for example selected from N, O and S.

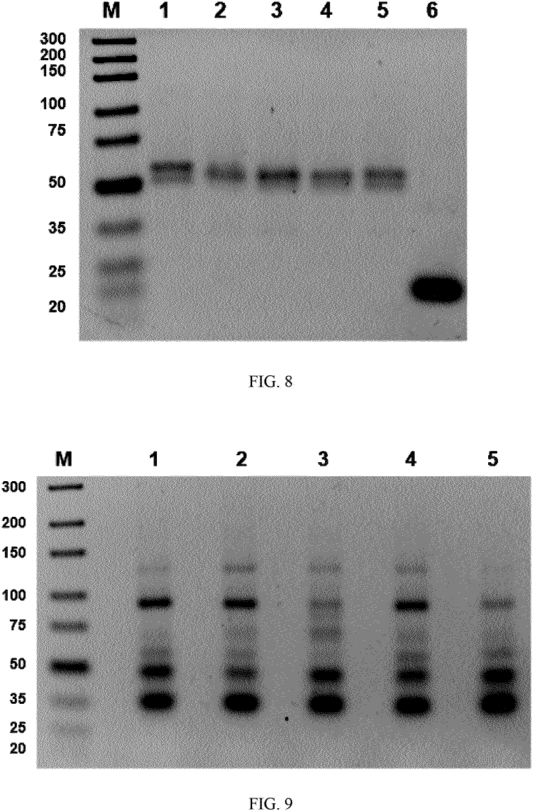

In various embodiments, the organic molecule used as a starter material, i.e. before it is subjected to a reaction as defined in step (2) of the methods, may be selected from the (bifunctional starting) compounds depicted in FIG. 7. It is understood that these compounds are then coupled to the nucleic acid tag by any suitable group and reaction.

The term “nucleic acid moiety”, as used herein, refers to a nucleic acid sequence that is covalently coupled to an organic moiety, as defined above, to form a conjugate molecule. The terms “nucleotide”, “nucleic acid molecule” or “nucleic acid sequence”, as interchangeably used herein, may relate to DNA (deoxyribonucleic acid) molecules, RNA (ribonucleic acid) molecules or molecules comprising both, DNA and RNA. Said molecules may appear independent of their natural genetic context and/or background. The term “nucleic acid molecule/sequence” further refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded form (helix). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms.

“RNA” or “ribonucleic acid”, as interchangeably used herein, relates to a chain of nucleotides wherein the nucleotides contain the sugar ribose and bases selected from the group of adenine (A), cytosine (C), guanine (G), or uracil (U). “DNA” or “deoxyribonucleic acid”, as interchangeably used herein, relates to a chain of nucleotides wherein the nucleotides contain the sugar 2′-deoxyribose and bases selected from adenine (A), guanine (G), cytosine (C) and thymine (T). The term “mRNA” refers to messenger RNA. If DNA is used, it is preferably double-stranded, with both strands preferably having the same length and being paired by Watson-Crick base-pairing. Irrespective of whether DNA or RNA or both are used, the respective indication identifies the backbone only, as both types of nucleic acid will comprise the modified nucleotides defined herein.

The term “base” or “nucleobase”, as interchangeably used herein, relates to nitrogen-containing biological compounds (nitrogenous bases) found linked to a sugar within nucleosides - the basic building blocks of deoxyribonucleic acid (DNA) and ribonucleic acid (RNA). Their ability to form base pairs and to stack upon one another lead directly to the helical structure of DNA and RNA. The primary, or canonical, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Uracil and thymine are identical except that uracil lacks the 5′ methyl group. Adenine and guanine belong to the double-ringed class of molecules called purines (abbreviated as R). Cytosine, thymine, and uracil are all pyrimidines (abbreviated as Y). Other bases that do not function as normal parts of the genetic code are termed non-canonical.

The modified and/or unmodified pyrimidine nucleobases that can be used to be coupled to the organic molecule to form a compound of the library include, but are not limited to, thymine, cytosine, uracil, 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, 1-methylpseudouridine, 3-methylcytidine, 5-methylcytidine, 5-methylaminomethyluridine, 5-methoxyaminomethyl-2-thiouridine, 5-methoxycarbonylmethyl-2-thiouridine, 5-methoxycarbonylmethyluridine, 5-methoxyuridine, uridine-5-oxyacetic acid-methylester, uridine-5-oxyacetic acid, pseudouridine, 2-thiocytidine, 5-methyl-2-thiouridine, 2-thiouridine, 4-thiouridine, 5-methyluridine, 2′-O-alkyluridine, 2′-O-alkylthymidine, 2′-O-alkylcytidine and 3-(3-amino-3-carboxy-propyl)uridine. Also possible are combinations thereof. In some embodiments, it may be advantageous to use unmodified pyrimidine bases while in other the use of modified ones may be indicated. According to various embodiments, these nucleobases are used in form of their corresponding nucleosides or nucleotides, typically the deoxynucleosides/deoxynucleotides.

The term “alkyl”, as used in the context of 2′-O-alkyluridine, 2′-O-alkylthymidine, 2′-O-alkylcytidine, refers to a saturated or unsaturated hydrocarbon containing 1-20 carbon atoms including both acyclic and cyclic structures (such as methyl, ethyl, propyl, isopropyl, butyl, isobutyl, sec-butyl, pentyl, hexyl, cyclopropyl, cyclobutyl, cyclopentyl, cyclohexyl, propenyl, butenyl). In various embodiments, it is lower alkyl, i.e. acyclic C₁-C₄ alkyl.

In various embodiments, the nucleic acid moiety that is coupled to the small organic molecule in the first labeling step consists of 2 to 20 nucleotides, preferably 3 to 18, 3 to 15, 3 to 12 or 3 to 10 nucleotides. The lower limit of the nucleic acid sequence at this stage may be 2, 3, 4 or 5 nucleotides. The upper limit is typically 12, 10 or 8, for example 8-12 nucleotides. The length should be selected such as to allow identification of each different conjugate/organic molecule. Said nucleic acid moiety may later be elongated by addition of further nucleotides to reach its final length, as defined above. Said nucleic acid moiety used in the first labeling step may comprise only the actual barcode/code sequence and may thus consist of 7-deazapurines and/or 7-deaza-8-azapurines, and, optionally, modified and/or unmodified pyrimidine nucleotides, to avoid depurination during the following reaction steps. Further sequence elements may then be added at a later stage during the elongation steps. This strategy provides for the specific structure of the nucleic acid moieties of the final conjugates, as explained above.

In various embodiments, the nucleic acid moiety in the final conjugate is at least 8, preferably at least 10, 12, 14, 15, 20, 25, 30 or more nucleotides in length. The upper limit is typically about 150 nucleotides in size, preferably up to 120, or up to 100, or up to 90, or up to 80. These size limitations do, in various embodiments, relate to the part of the nucleic acid that serves as an identifier (barcode). In addition to those, the nucleic acid moiety in the final conjugate may also comprise primer binding sites to allow amplification and/or sequencing and, optionally, other sequence elements necessary for its intended use or its generation. In various embodiments, the barcode part of the sequence may be 8 to 50 nucleotides in length, for example 8 to 40 nucleotides, while the remainder of the total length are other sequence elements not used for barcoding. The upper limits recited above are thus preferably also the upper limits for total size of the nucleic acid moiety including all sequence elements necessary for its intended use that are not the actual barcode sequence.

In various embodiments, the code sequence may, for example, comprise adaptor sequences at its 5′ or 3′ end, preferably on both, that allow ligation of further code sequences and/or primer sequences.

In various embodiments, the nucleic acid moiety is RNA, DNA or a mixture thereof. In more preferred embodiments, the nucleic acid portion is DNA. The DNA is preferably double-stranded, as defined above. If the DNA is double-stranded, all organic reactions and the like, i.e. the synthesis of the conjugate may be performed with a single-stranded nucleic acid moiety and the counterstrand be synthesized only after all organic reactions, i.e. the reactions of the organic moiety, are completed.

In further embodiments, a compound library may include each conjugate molecule that has a different organic molecule from the plurality of conjugate molecules, with each organic molecule identified by a specific nucleic acid moiety. Basically, the methods for producing such a library are designed to ensure that each molecule of the plurality of conjugate molecules comprises a nucleic acid moiety that is distinct from the nucleic acid moieties of the other conjugate molecules: to achieve this, in a first step, all individual types of organic moieties are coupled to individual sequences of 7-deazapurine and/or 7-deaza-8-azapurine nucleotides and, optionally, modified/unmodified pyrimidines. By this method step, the different conjugate molecules can be discriminated from each other directly after the coupling to the individual nucleic acid sequences. These sequences may include or be adaptor nucleic acids that are suitable for subsequent elongation/reaction steps that affect the nucleic acid moiety. The nucleic acid sequence may be further elongated with additional nucleotides after the synthesis steps have been applied on the organic molecule. The nucleotides used in this elongation step of the adaptor nucleic acid can include pyrimidine nucleotides and purine nucleotides, both of which can be modified and/or unmodified (with unmodified purine nucleotides only being used if no further synthesis steps are performed on the organic molecule/group). The elongation step is used to individualize the nucleic acid moiety sequence attached to each individual organic molecule after it has been subjected to a reaction that led to its modification. The nucleic acid sequence added in the elongation step is thus an identifier for the respective preceding reaction/synthesis step of the organic group/moiety and together with the original individual nucleic acid sequence identifier of the starting organic moiety thus creates a unique identifier tag. A preferred method for the elongation of the nucleic acid moiety is DNA ligation with a ligase, such as T4 ligase, or with a chemical condensing agent, both strategies using nucleotides that comprises a nucleic acid sequence that hybridizes with the adaptor nucleic acid and that additionally comprises a nucleic acid sequence which encodes for a sequence that allows the individualization of the conjugate molecule (“second or further code sequence”). The adaptor sequences disclosed herein that may be present in the first code sequence, for example as flanking sequences, thus allow hybridization of a second nucleic acid molecule that comprises further sequence elements, such as a second or further code sequence or element necessary for functionality, and subsequently ligation in the elongation step. The to-be-ligated sequence may be a partly single-stranded hairpin structure, with the single-stranded part being used for hybridization to the nucleic acid moiety already present on the conjugate. The elongation step is thus in various embodiments a ligation step that uses a ligase to attach a further nucleotide sequence to the nucleic acid moiety of the conjugate. It has been surprisingly found that ligases, such as T4 ligase, can carry out the ligation reaction also for the modified sequences used herein for barcoding, i.e. in particular the 7-deazapurine and/or 7-deaza-8-azapurine nucleotides.

“Covalent” or “covalent bond”, as used herein, refers to a chemical bond that involves the sharing of electron pairs between atoms. These electron pairs are known as shared pairs or bonding pairs and the stable balance of attractive and repulsive forces between atoms when they share electrons is known as covalent bonding. For many molecules, the sharing of electrons allows each atom to attain the equivalent of a full outer shell, corresponding to a stable electronic configuration. Covalent bonding includes many kinds of interactions, including σ-bonding, π-bonding, metal-to-metal bonding, agnostic interactions, bent bonds, and three-center two-electron bonds.

In various embodiments, the conjugate molecules of the library further comprise a linker portion between the nucleic acid moiety and the organic moiety molecule. In various embodiments, this linker group may be a polyoxyalkylene moiety or an alkylene moiety, in some embodiments this linker portion is polyethylene glycol (PEG), comprising, for example, 3 (PEG₃) or 4 (PEG₄) ethylene glycol units. Further linker groups are described below. The linkers are also referred to as “chemical linkers” to distinguish over nucleotide linkers. Chemical linkers may also be incorporated into the assembly reactants. The chemical linkers may be included between any of the moieties. Chemical linkers may optionally connect two or more of the moieties to introduce additional functionality or facilitate synthesis. The chemical linker can be a bond between any of the moieties. The bond can include a physical interaction, such as chemical bonds (either directly linked or through intermediate structures), or a non-physical interaction or attractive force, such as electrostatic attraction, hydrogen bonding, and van der Waals/dispersion forces. The linker may be conjugated to any given nucleotide within the nucleic acid moiety. In addition, any position of the ribose or the nucleobase within a given nucleotide of the nucleic acid moiety may be used to the coupling of the linker. In various embodiments, the linker is coupled to the nucleic acid moiety by the free 5′-position, namely a phosphate group, of the most 5′-terminal nucleotide of the nucleic acid moiety. Alternatively, it may also be coupled to the 5′ penultimate nucleotide. Such coupling to a nucleotide close to one of the ends, in particular the 5′ end, for example the 5′-penultimate nucleotide, may even be preferred in various embodiments. For example, ethynyl-dU (i.e. dT where the 5-methyl group is replaced by the ethynyl or other alkynyl group) may be used as the penultimate 5′-terminal nucleotide. As described below, such an alkynyl group allows attachment of linker groups by using alkynyl-azide cyclization reaction. The nucleotide used for attachment of the linker group and the organic moiety is typically referred to herein, in the examples, as T*.

The chemical linkers may aid in facilitating spatial separation of the moieties, increasing flexibility of the moieties relative to each other, introducing a cleavage site or modification site to the templated assembly reactant, facilitating synthesis of the templated assembly reactant, improving physical or functional characteristics (such as solubility, hydrophobicity, charge, cell-permeability, toxicity, biodistribution, or stability) of a templated assembly reactant, or any combination of the above. In various embodiments, the chemical linker is derived from a crosslinker that facilitates connecting the assembly reactant moieties via bioconjugation chemistry. “Bioconjugation chemistry”, as used herein, refers to the chemical synthesis strategies and reagents that ligate common functional groups together under mild conditions, facilitating the modular construction of multi-moiety compounds. Due to the mild reaction conditions, bioconjugate chemistry approaches can be suitable for ligating biomolecules, such as nucleic acids, peptides, or polysaccharides. Some non-limiting examples can include chains of one or more of the following: alkyl groups, alkenyl groups, amides, esters, thioesters, ketones, ethers, thioethers, disulfides, ethylene glycol, cycloalkyl groups, benzyl groups, heterocyclic groups, maleimidyl groups, hydrazones, urethanes, azoles, imines, haloalkyl groups, carbamates, or combinations of any of these. However, since the starting nucleic acid moiety is designed to withstand harsh reaction conditions, alternative conjugation chemistries may also be used. In some embodiments, the linker is polyethylene glycol (PEG).

Generally, suitable linker chemistries are known to those skilled in the art and some are exemplified in the examples section. For example, the nucleic acid moiety may comprise an alkyne group, such as a ethynyl or propynyl group, which is reacted with a linker, such as a PEG linker group, functionalized with an azide (N₃) group. The alkynyl and azide group react to form a triazol ring that links the nucleic acid moiety to the linker group, such as the PEG linker group, for example (PEG)₃ or (PEG)₄, i.e. 3 or 4 linked ethylene glycol units, which is turn is functionalized on the other end to allow attachment of the organic moiety. Said functionalization may, for example, beNH-Fluorenylmethoxycarbonyl (Fmoc).

In addition to chemical linkers between moieties, additional functionality may optionally be introduced to the conjugate molecule by the addition of accessory groups to the moieties. Some non-limiting examples of accessory groups can include appending a chemical tag or fluorophore to track the location of the conjugation molecule, or appending an agent that improves delivery of the conjugate molecule to a given target, stabilizing groups or groups that improve purification of the conjugate molecule.

During any of the synthesis steps described herein, including the synthesis of the starting conjugate or the synthesis/modification of the organic moiety, the nucleic acid moiety/conjugate may be coupled to a solid support. Said coupling may be achieved via the 5′ end of the nucleic acid moiety. The solid support may be controlled pore glass (CPG).

“At least one”, as used herein, relates to one or more, in particular 1, 2, 3, 4, 5, 6, 7, 8, 9, 10 or more.

In a further aspect, a method may be used for synthesizing a compound library, wherein said library comprises a plurality of conjugate molecules, wherein said conjugates comprise a small organic molecule covalently coupled to a nucleic acid moiety, the synthesis of each conjugate molecule comprises: reacting a nucleic acid consisting of 7-deazapurines and/or 7-deaza-8-azapurines, and, optionally, modified and/or unmodified pyrimidine nucleotides with an organic molecule under conditions that allow the conjugation of said molecules, wherein each conjugate molecule has a different organic molecule and a different nucleic acid label that identifies its corresponding organic molecule. As further detailed above, the nucleic acid moiety attached in this first step is also referred to as starting nucleic acid sequence identifier and is distinguished over any other nucleotide stretches later added by elongation of the nucleic acid moiety in that is consists of 7-deazapurines and/or 7-deaza-8-azapurines, and, optionally, modified and/or unmodified pyrimidine nucleotides and does not contain any unmodified purine nucleotides. The respective nucleotide sequence is of a length that allows identification of the starting organic group. In various embodiments, said starting nucleic acid moiety is therefore 3 to 18, 4 to 15, 4 to 12 or 5 to 10 nucleotides in length. The lower limit of the nucleic acid sequence at this stage may be 3, 4 or 5 nucleotides. The upper limit is typically 12, 11, 10, 9 or 8, for example 8-12 nucleotides.

As described above, the method for synthesizing a compound library may comprise the following steps: (1) reacting a nucleic acid moiety/sequence consisting of 7-deazapurines and/or 7-deaza-8-azapurines and, optionally, modified and/or unmodified pyrimidine nucleotides, typically 3 to 18 nucleotides in length and not comprising any unmodified purine nucleotides, with an organic molecule under conditions that allow the conjugation of said molecules; (2) subjecting the conjugate obtained in step (1) to further reactions to modify the small organic molecule, typically reaction conditions that would result in depurination of unmodified purine nucleotides; and (3) elongating the nucleic acid moiety of the conjugate obtained in step (2) by a further nucleic acid sequence. In step (1) of said method, each organic molecule is conjugated to a unique nucleic acid molecule that allows its identification. This means that the nucleic acid molecule has a certain length as defined below, typically at least 3, preferably at least 4, or more preferably at least 5 nucleotides. To avoid that the nucleic acid moiety is affected by the reaction in step (2), the nucleic acid moiety in step (1) consists of 7-deazapurines and/or 7-deaza-8-azapurines, and, optionally, modified and/or unmodified pyrimidine nucleotides, i.e. does not contain unmodified purine bases. In some embodiments, the nucleic acid sequence added in step (3) can comprise modified and/or unmodified purine and pyrimidine nucleotides, as the reaction in step (2) is already completed. In various embodiments, it is however also possible to repeat steps (2) and (3) multiple times. This allows synthesis of more complex organic molecules. In such embodiments, the elongation step (3) is carried out again with a further nucleic acid sequence that has the same restrictions as the starting nucleic acid sequence, i.e. consists of 7-deazapurines and/or 7-deaza-8-azapurines and, optionally, modified and/or unmodified pyrimidine nucleotides, typically 3 to 18 nucleotides in length and does not comprise any unmodified purine nucleotides. Only the last elongation steps after all cycles of step (2) have been completed may use a further nucleic acid sequence comprising modified or unmodified purine nucleotides other than 7-deazapurines and/or 7-deaza-8-azapurines. This means, depending on the type of subsequent reaction the conjugate is subjected to after the first step (3), this requires that the elongation in step (3) is carried out using a suitable nucleic acid sequence, e.g. a nucleic acid sequence that consists of 7-deazapurines and/or 7-deaza-8-azapurines, and, optionally, modified and/or unmodified pyrimidine nucleotides but no unmodified purine nucleotides, and only after the final cycle of reaction step (2) is completed, the elongation may be carried out with unmodified purine and, optionally pyrimidine bases.

The term “synthesizing” or “synthesis”, as used herein, refers to a purposeful execution of chemical reactions to obtain a product, or several products. This happens by physical and chemical manipulations usually involving one or more reactions. This may imply that the process is reproducible, reliable, and established to work in multiple laboratories. This term is predominantly used herein to refer to the synthesis of the starting conjugate from an organic molecule and a starting nucleic acid moiety as well as to the synthesis of the desired final organic moiety of the conjugate from a starting organic group present in the starting conjugate (reagent/reactant/educt) by a sequence of one or more reaction steps.

A chemical synthesis begins by selection of compounds that are known as reagents or reactants or educts. Various reaction types can be applied to these to synthesize the product, or an intermediate product. This requires mixing the compounds in a reaction vessel, such as but not limited to 96-well plate, an Eppendorf tube, etc., a chemical reactor or a simple round-bottom flask. Many reactions require some form of work-up procedure before the final product is isolated. The amount of product in a chemical synthesis is the reaction yield. Typically, chemical yields are expressed as a weight in grams (in a laboratory setting) or as a percentage of the total theoretical quantity of product that could be produced. A side reaction is an unwanted chemical reaction taking place that diminishes the yield of the desired product.

“Reacting” as used with regard to the method for synthesizing a compound library refers to contacting the educts under conditions that allow formation of the product, namely the conjugate molecule. While the coupling step of organic starting group and nucleic acid starting moiety may also be considered to be such a reaction, synthesis of the final conjugate moiety mostly refers to the modification of the organic moiety of the starting conjugate to obtain the desired final conjugate of the compound library. Exemplary reactions and reaction conditions are described below and in the examples. The reactions are typically carried out under conditions that require the specific nucleic acid moieties described herein to avoid that these are also affected and modified by the reaction carried out.

In various embodiments of the method, the conjugate molecule obtained by coupling a nucleic acid moiety (starting nucleic acid moiety) to a starting organic molecule, i.e. the educt conjugate, or in any previous reaction steps, for example as described above, is (further) used in a reaction comprising a) a strong acid, preferably an acid having a pKₐ of less than 3; and/or b) a catalyst selected from the group consisting of zinc, copper, silver, gold, ruthenium, iron, osmium, cobalt, rhodium, iridium, nickel, palladium, platinum, ytterbium, and derivatives thereof, preferably zinc, copper, silver, gold, ruthenium, ytterbium and derivatives thereof. Said reactions may be those referred to as step (2) above. This means that these reactions are those intended to modify or synthesize the desired organic molecule of the conjugate compound which may adversely affect integrity of a nucleic acid identifier sequence comprising unmodified purine nucleotides, thus being responsible for the requirement that the nucleic acid moiety used for identification of the conjugated subjected to said reaction is as defined herein, i.e. consists of of 7-deazapurines and/or 7-deaza-8-azapurines and, optionally, modified and/or unmodified pyrimidine nucleotides.

In various embodiments of the method, the conjugate molecule is used, for example, in a reaction involving any one or more of the following, without being limited thereto: a Povarov reaction, for example one that leads to densely substituted tetrahydroquinolines; an aza-Diels-Alder reaction; a Petasis reaction; an Ugi reaction; an Ugi azide reaction; an Ugi-aza-Wittig reaction, for example leading to oxadiazoles; a Grobke-Blackburn-Bienayme reaction; a Cu(II) promoted “SnAP” chemistry reaction; aminoindolizine, pyrazoline and/or pyrazole syntheses by Au(I) catalysis; an In(III)-mediated reaction, for example one that uses carbohydrates for heterocycle synthesis, and metal-promoted aldehyde-alkyne-amine three-component reactions.

The strength of an acid refers to its ability or tendency to lose a proton (H⁺). A strong acid is one that completely ionizes (dissociates) in a solution. In water, one mole of a strong acid HA dissolves yielding one mole of H⁺ (as hydronium ion H₃O⁺) and one mole of the conjugate base, A⁻. Essentially, none of the non-ionized acid HA remains. Examples of strong acids are hydrochloric acid (HCl), hydroiodic acid (HI), hydrobromic acid (HBr), perchloric acid (HClO₄), nitric acid (HNO₃), trifluoroacetic acid, trichloroacetic acid, and sulfuric acid (H₂SO₄). In aqueous solution, each of these essentially ionizes 100%. In contrast, a weak acid only partially dissociates. Examples in water include carbonic acid (H₂CO₃) and acetic acid (CH₃COOH). At equilibrium, both the acid and the conjugate base are present in solution. Stronger acids have a larger acid dissociation constant (Kₐ) and a smaller logarithmic constant (pKₐ = -log Kₐ) than weaker acids. Strong acids typically have a pKₐ <1.74.

The term “transition metal”, as used herein, is a synonym for elements of the groups 3 to 12 of modern IUPAC numbering. Examples of transition metals are copper (Cu), silver (Ag), and gold (Au) and other described herein.

“Catalyst”, as used herein, refers to any substance that increases the rate of a reaction without itself being consumed. In general, catalytic action is a chemical reaction between the catalyst and a reactant, forming chemical intermediates that are able to react more readily with each other or with another reactant, to form the desired end product. During the reaction between the chemical intermediates and the reactants, the catalyst is regenerated. The modes of reactions between the catalysts and the reactants vary widely. Typical of these reactions are acid-base reactions, oxidation-reduction reactions, formation of coordination complexes, and formation of free radicals.

In various embodiments, the afore-mentioned reaction conditions require that the nucleic acid moiety of the conjugate consists of 7-deazapurines and/or 7-deaza-8-azapurines and, optionally, modified and/or unmodified pyrimidine nucleotides, since unmodified purine bases have been shown to be adversely affected by said reaction conditions (Potowski et al., supra). Therefore, any elongation that took place before the conjugate is subjected to reaction conditions to modify the organic moiety is carried out using the appropriate nucleotides or nucleic acid sequences, i.e. nucleic acid sequences that consist of 7-deazapurines and/or 7-deaza-8-azapurines and, optionally, modified and/or unmodified pyrimidine nucleotides. Only after the organic moiety has undergone its final reaction, the last elongation step can also be carried out with DNA barcode oligonucleotides containing unmodified purine nucleotides and, optionally, pyrimidine nucleotides.

In various embodiments of the method, the nucleic acid moiety is therefore elongated in an additional reaction step with pyrimidine and/or purine nucleotides after the conjugation step. As described above, the elongation of the nucleic acid molecule may be put into practice by using PCR and a primer nucleic acid molecule, which comprises a nucleic acid sequence that hybridizes with the adaptor nucleic acid and a nucleic acid sequence which comprises or encodes for a sequence that allows the individualization of the conjugate molecule.

It was surprisingly found that 7-deazapurines and/or 7-deaza-8-azapurines are not degraded under conditions used for the synthesis of heterocyclic chemical compounds (for example reactions involving the use of transition metals as catalysts or strong (protic) acids). However, the variety of chemical reactions carried out on the organic molecule is not limited to reactions that are only tolerated by 7-deazapurines and/or 7-deaza-8-azapurines nucleotides. The chemical reactions have already been listed above and may comprise, but are not limited to, Povarov reaction, aza-Diels-Alder reaction, Petasis reaction, Ugi reaction, Ugi azide reaction, Ugi-aza-Wittig reaction leading to oxadiazoles, Grobke-Blackburn-Bienayme reaction, Tin Amine Protocol (SnAP) chemistry, aminoindolizine, pyrazole and/or pyrazoline synthesis by Au(I) catalysis, In(III)-mediated reactions, for example those that use carbohydrates for heterocycle synthesis, and metal-promoted aldehyde-alkyne-amine three-component reactions. This variety of chemical reactions may also include, without limitation, amide synthesis, stepwise substitution of cyanuric chloride, protective group chemistry, amide bond formation, carbonyl reactions, such as synthesis of diamine-containing cyclic or linear compounds, aromatic substitutions, such as nucleophilic aromatic substitutions of reactive heteroaromatic halides, cross-coupling reactions, such as palladium-catalyzed synthesis of biaryl compounds, Diels-Alder reactions, benzimidazole formation, macrocycle synthesis, substitution with N-nucleophiles, nucleophilic substitution of reactive aliphatic halides, Cu(I)-catalyzed alkyne-azide cycloaddition, Horner-Wadsworth-Emmons reaction with reactive phosphonium ylids, reduction of aromatic nitro groups and appendage of cyanuric chloride.

In a third aspect, a compound library may be used for screening a compound binding to a target molecule.

The term “screening”, as used herein, means that a plurality of candidate compounds (this can be an amount of 10 or less compounds or up to 10.000.000 compounds or more) can be screened in a single experiment for their ability to bind to a given target molecule. In various embodiments, 10 or more, 20 or more, 50 or more, 100 or more, 1000 or more, 1500 or more, 2000 or more, 5000 or more or 10000 or more of the candidate compounds are pooled in a single vessel and tested for their ability to bind to target molecule. Thus, said candidate compounds pooled in a single vessel and exposed to the target molecule compete for binding to said target molecule. Upon being brought into contact with the target molecule, the candidate compounds may bind to the target molecule or being washed out by one or more washing steps. Subsequently, the candidate compounds bound to the target molecule can be identified via their individual DNA label. Methods to identify candidate compounds based on their DNA label are described herein and are well-known in the art.

A “target molecule”, as used herein, may be any molecule which can be bound by a binding molecule and may for example be a biological substance such as a biomolecule, complexes, cell fractions or cells. Preferably, a target molecule is a protein and/or a nucleic acid, e.g. DNA, or RNA molecule. Even more preferred are target molecules such as a peptide, a protein, a drug molecule, a small molecule, or a vitamin. In some embodiments, a “target molecule” may be a biomarker. A target molecule may also refer to a biological molecule that is either naturally present in a cell or subject or has been previously introduced into a cell or subject. A “target molecule” can be any polypeptide, lipid, nucleic acid, small molecule, saccharide or a vitamin in, on or of the cell. It may be located in the nucleus, nucleolus, cytoplasm, mitochondria, Golgi apparatus, endoplasmic reticulum or membrane of the cell. In various embodiments, the target molecules of the methods are polypeptide molecules. In various embodiments, the target molecule is a protein.

The term “protein”, as used herein, relates to one or more associated polypeptides, wherein the polypeptides consist of amino acids linked by peptide (amide) bonds. The term polypeptide refers to a polymeric compound comprised of covalently linked amino acid residues. The amino acids are preferably the 20 naturally occurring amino acids glycine, alanine, valine, leucine, isoleucine, phenylalanine, cysteine, methionine, proline, serine, threonine, glutamine, asparagine, aspartic acid, glutamic acid, histidine, lysine, arginine, tyrosine and tryptophan.

Suitable targets may include one or more of peptides, proteins (e.g., antibodies, affibodies, or aptamers), nucleic acids (e.g., polynucleotides, DNA, RNA, or aptamers); polysaccharides (e.g., lectins or sugars), lipids, enzymes, enzyme substrates, ligands, receptors, antigens, or haptens. One or more of the aforementioned targets may be characteristic for particular cells, while other targets may be associated with a particular disease or condition. In some embodiments, targets in a tissue sample that may be detected and analyzed using the methods disclosed herein may include, but are not limited to, prognostic targets, hormone or hormone receptor targets, lymphoid targets, tumor targets, hematopoietic targets, cell cycle associated targets, neural tissue and tumor targets, or cluster differentiation targets.

Suitable examples of prognostic targets may include enzymatic targets such as galactosyl transferase II, neuron specific enolase, proton ATPase-2, or acid phosphatase.

Suitable examples of hormone or hormone receptor targets may include human chorionic gonadotropin (HCG), adrenocorticotropic hormone, carcinoembryonic antigen (CEA), prostate-specific antigen (PSA), estrogen receptor, progesterone receptor, androgen receptor, gClq-R/p33 complement receptor, IL-2 receptor, p75 neurotrophin receptor, PTH receptor, thyroid hormone receptor, or insulin receptor.

Suitable examples of hematopoietic targets may include CD45, CD34, CD133, HLA-DR, CD115, CD116, CD117, CD33, CD38, CD90, CD71, Ki67, Flt3, CD163, CD45RA, CD3, IgD, CD105, CD45, and also c-kit, - the receptor for stem cell factor.

Suitable examples of lymphoid targets may include alpha-1-antichymotrypsin, alpha-1-antitrypsin, B cell target, bcl-2, bcl-6, B lymphocyte antigen 36 kD, BMl (myeloid target), BM2 (myeloid target), galectin-3, granzyme B, HLA class I Antigen, HLA class II (DP) antigen, HLA class II (DQ) antigen, HLA class II (DR) antigen, human neutrophil defensins, immunoglobulin A, immunoglobulin D, immunoglobulin G, immunoglobulin M, kappa light chain, kappa light chain, lambda light chain, lymphocyte/histocyte antigen, macrophage target, muramidase (lysozyme), p80 anaplastic lymphoma kinase, plasma cell target, secretory leukocyte protease inhibitor, T cell antigen receptor (JOVI 1), T cell antigen receptor (JOVI 3), terminal deoxynucleotidyl transferase, or unclustered B cell target.

Suitable examples of tumor targets may include alpha fetoprotein, apolipoprotein D, BAG-I (RAP46 protein), CA19-9 (sialyl lewisa), CA50 (carcinoma associated mucin antigen), CAl 25 (ovarian cancer antigen), CA242 (tumor associated mucin antigen), chromogranin A, clusterin (apolipoprotein J), epithelial membrane antigen, epithelial-related antigen, epithelial specific antigen, gross cystic disease fluid protein- 15, hepatocyte specific antigen, heregulin, human gastric mucin, human milk fat globule, MAGE-I, matrix metalloproteinases, melan A, melanoma target (HMB45), mesothelin, metallothionein, microphthalmia transcription factor (MITF), Muc-1 core glycoprotein. Muc-1 glycoprotein, Muc-2 glycoprotein, Muc-5AC glycoprotein, Muc-6 glycoprotein, myeloperoxidase, Myf-3 (Rhabdomyosarcoma target), Myf-4 (Rhabdomyosarcoma target), MyoDl (Rhabdomyosarcoma target), myoglobin, nm23 protein, placental alkaline phosphatase, prealbumin, prostate specific antigen, prostatic acid phosphatase, prostatic inhibin peptide, PTEN, renal cell carcinoma target, small intestinal mucinous antigen, tetranectin, thyroid transcription factor- 1, tissue inhibitor of matrix metalloproteinase 1, tissue inhibitor of matrix metalloproteinase 2, tyrosinase, tyrosinase-related protein-1, villin, or von Willebrand factor.

Suitable examples of cell cycle associated targets may include apoptosis protease activating factor- 1, bcl-w, bcl-x, bromodeoxyuridine, CAK (cdk-activating kinase), cellular apoptosis susceptibility protein (CAS), caspase 2, caspase 8, CPP32 (caspase-3), CPP32 (caspase-3), cyclin dependent kinases, cyclin A, cyclin Bl, cyclin Dl, cyclin D2, cyclin D3, cyclin E, cyclin G, DNA fragmentation factor (N-terminus), Fas (CD95), Fas-associated death domain protein, Fas ligand, Fen-1, IPO-38, McI-I, minichromosome maintenance proteins, mismatch repair protein (MSH2), poly (ADP- Ribose) polymerase, proliferating cell nuclear antigen, pl6 protein, p27 protein, p34cdc2, p57 protein (Kip2), pi 05 protein, Stat 1 alpha, topoisomerase I, topoisomerase II alpha, topoisomerase III alpha, or topoisomerase II beta.

Suitable examples of neural tissue and tumor targets may include alpha B crystallin, alpha-internexin, alpha synuclein, amyloid precursor protein, beta amyloid, calbindin, choline acetyltransferase, excitatory amino acid transporter 1, GAP43, glial fibrillary acidic protein, glutamate receptor 2, myelin basic protein, nerve growth factor receptor (gp75), neuroblastoma target, neurofilament 68 kD, neurofilament 160 kD, neurofilament 200 kD, neuron specific enolase, nicotinic acetylcholine receptor alpha4, nicotinic acetylcholine receptor beta2, peripherin, protein gene product 9, S-100 protein, serotonin, SNAP-25, synapsin I, synaptophysin, tau, tryptophan hydroxylase, tyrosine hydroxylase, or ubiquitin.

Suitable examples of cluster differentiation targets may include CDIa, CDIb, CDIc, CDId, CDIe, CD2, CD3delta, CD3epsilon, CD3gamma, CD4, CD5, CD6, CD7, CD8alpha, CD8beta, CD9, CDlO, CDl Ia, CDl Ib, CDl Ic, CDwl2, CD13, CD14, CD15, CD15s, CD16a, CD16b, CDwl7, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD44R, CD45, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD65s, CD66a, CD66b, CD66c, CD66d, CD66e, CD66f, CD68, CD69, CD70, CD71, CD72, CD73, CD74, CDw75, CDw76, CD77, CD79a, CD79b, CD80, CD81, CD82, CD83, CD84, CD85, CD86, CD87, CD88, CD89, CD90, CD91, CDw92, CDw93, CD94, CD95, CD96, CD97, CD98, CD99, CDlOO, CDlOl, CD102, CD103, CD104, CD105, CD106, CD107a, CD107b, CDwlO8, CD109, CDl 14, CDl 15, CDl 16, CDl 17, CDwI 19, CD120a, CD120b, CD121a, CDwl21b, CD122, CD123, CD124, CDwl25, CD126, CD127, CDwl28a, CDwl28b, CD130, CDwl31, CD132, CD134, CD135, CDwl36, CDwl37, CD138, CD139, CD140a, CD140b, CD141, CD142, CD143, CD144, CDwl45, CD146, CD147, CD148, CDwl49, CDwl50, CD151, CD152, CD153, CD154, CD155, CD156, CD157, CD158a, CD158b, CD161, CD162, CD163, CD 164, CD 165, CD 166, and TCR-zeta.

Other suitable prognostic targets hormone or hormone receptor targets lymphoid targets tumor targets cell cycle associated targets neural tissue and tumor targets include centromere protein-F (CENP-F), giantin, involucrin, lamin A&C (XB 10), LAP-70, mucin, nuclear pore complex proteins, pi 80 lamellar body protein, ran, cathepsin D, Ps2 protein.

The term “contacting”, as used herein in the context of conjugate molecules and target molecules, refers generally to providing access of one component, reagent, analyte or sample to another. For example, contacting can involve mixing a solution comprising a conjugate molecule and a target molecule. The solution comprising one component, reagent, analyte or sample may also comprise another component or reagent, such as dimethyl sulfoxide (DMSO) or a detergent, which facilitates mixing, interaction, uptake, or other physical or chemical phenomenon advantageous to the contact between components, reagents, analytes and/or samples.

The term “identify”, as used herein in the context of screening, relates to the recognition of a conjugate molecule that has the ability to completely or partially bind to the biological target molecule, and/or completely or partly revert molecular changes of a cell induced by pathological conditions or to inhibit or activate enzymatic functions.

The term “sequence”, as used herein, relates to the primary nucleotide sequence of nucleic acid molecules or the primary amino acid sequence of a protein.

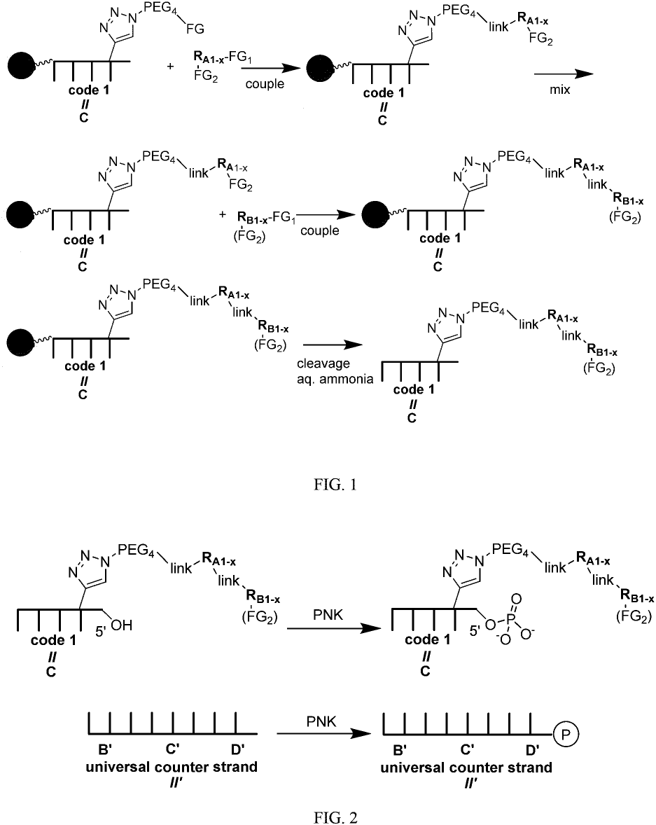

FIG. 1 shows the synthesis of conjugates on controlled pore glass-coupled DNA barcodes (two or three-cycle library). The controlled pore glass (CPG) is schematically shown as a black dot. The designation of the sequence code 1 may be found in Tables 3-8. In the methods and conjugates, said code 1 is an oligonucleotide consisting of 7-deazapurines and/or 7-deaza-8-azapurines and, optionally, modified and/or unmodified pyrimidine nucleotides. Said code 1 identifies the first building block R_{A1-x} and may after the reaction depicted in FIG. 1 is completed be extended to further identify the moiety resulting from the addition of the second building block, R_{Bx-1}. This allows coupling the first building block in different reactions with different second building blocks and use elongation by different nucleotide sequences for the different second building blocks used. If only one second building block is used, the nucleic acid moiety of code 1 may also serve to identify the reaction product of first and second building blocks without further elongation.

The CPG-bound oligonucleotide has a linker, the 1,2,3-triazole-PEG₄- moiety between the nucleic acid moiety and a functional group ‘FG’. A building block R_{A1-x} coupled to FG₁ and FG₂ is then added to the CPG-bound oligonucleotide where FG₁ reacts with FG to form a ‘link’ between the PEG and the building block R_{A1-x}. A second building block is coupled similarly to the first building block where the FG₁ of the second building block reacts with FG₂ of the first building block to form a ‘link’ between the first building block and the second building block. The second building block has an optional second functional group, FG₂, in particularly if it is intended to further modify it by another cycle of reaction. Two building blocks (R_{A1-x} and R_{B1-} ₓ) are depicted here (known as a 2-cycle library), but a 3-cycle library that further comprises R_{CX-1} coupled via its functional group FG₁ to FG₂ of the second building block is also possible. Up to 5 building blocks, i.e. a 5-cycle library may also be useful with the compound library disclosed herein. The controlled pore glass is then cleaved with aqueous ammonia after the addition of building blocks is complete.

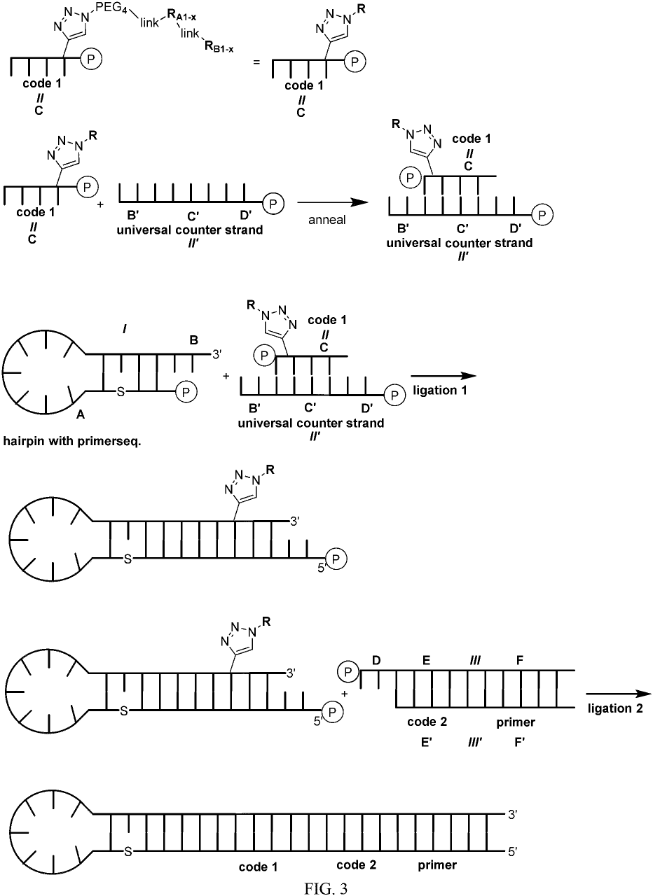

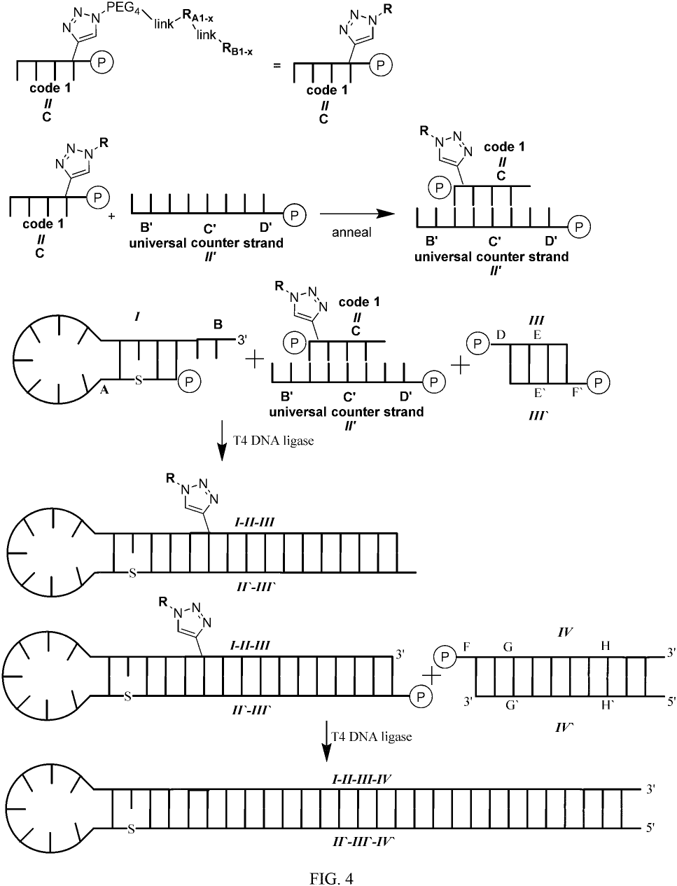

FIG. 2 shows the enzymatic phosphorylation of chemically modified code 1 DNA sequences II and degenerate counter strand II′ using a polynucleotide kinase (PNK). Once the building blocks have been added, i.e. the organic moiety has been synthesized, it is phosphorylated at the 5′-end with a polynucleotide kinase, such as T4 PNK (as a non-limiting example for a suitable kinase). A universal counter strand II′ is depicted that is also phosphorylated at its 5′-end. The DNA barcode sequence has the sequence II (C) and the universal counter strand has the sequence II′ and comprises a sequence stretch C′ that can hybridize to the C sequence in the barcode identifier as well as two flanking sequences B′ and D′. These flanking sequences form an overhang to create sticky ends and allow further elongation, annealing and/or ligation (See, e.g., FIG. 3).

The universal counter strand II′ is then annealed to the oligonucleotide sequence with the building blocks as depicted in FIG. 3. In FIG. 3 the linked first and second building blocks are, for ease of representation, only shown as “R”. FIG. 3 illustrates a two-cycle encoding strategy for short single-stranded DNA conjugates. The designation of the sequences may be found in Tables 3-8. A primer sequence is then added (here depicted as a hairpin primer sequence comprising a C₉ spacer referred to as “S”) that comprises a nucleotide sequence B that can hybridize to the B′ sequence of the universal counter strand to allow elongating the annealed sequence. After annealing and ligation of said primer, a second elongation (e.g. ligation) occurs to add a III/III′ double-stranded oligonucleotide comprising a sticky end sequence D that hybridizes to the D′ overhang, a code 2 sequences (E/E′) and a primer sequence (F/F′). Such elongation may be repeated and occur as many times as desired by one skilled in the art. The elongation may occur with additional nucleotides that may be pyrimidines and/or purines, both of which may be modified and/or unmodified. For example, the pyrimidines and/or purines of the hairpin with primer sequence and/or primer of code 2 may be modified and/or unmodified in an embodiment. The elongation of the sequence allows for individualization of the nucleic acid moiety sequence attached to each individual organic molecule. In a non-limiting embodiment, T4 ligase and/or a chemical condensing agent may be used for elongation purposes.

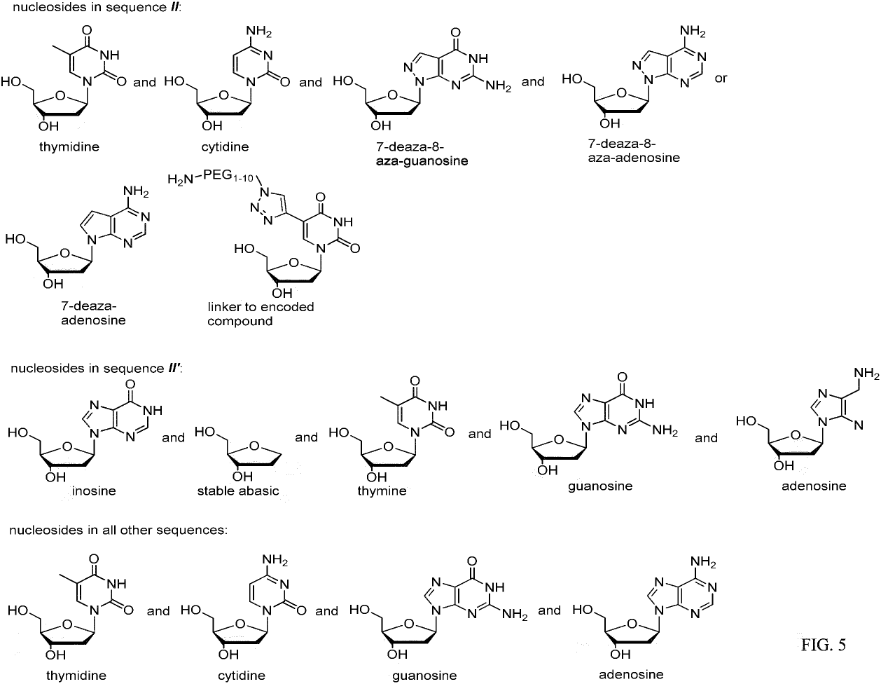

FIG. 4 shows a hairpin encoding strategy for short single-stranded DNA conjugates. The designation of the sequences may be found in Tables 3-8. The process depicted in FIG. 4 is similar to that of FIG. 3 but differs in that two additional sequences are ligated to the annealed strand comprising the phosphorylated code 1 strand and the phosphorylated universal counter strand II′.

More specific examples of the above procedures are described below.

### EXAMPLES

### Example 1a: Synthesis of the 7-deazaadenine Building Block for Automated DNA Synthesis

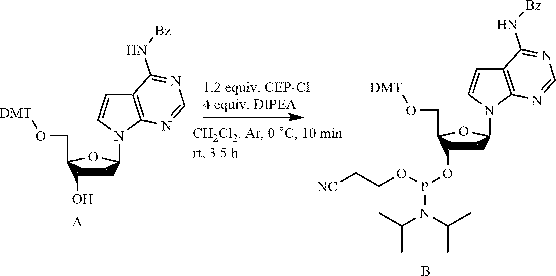

N⁶-Benzoyl-2′-deoxy-5′-O-DMT-7-deaza-2′-deoxyadenosine 3′-CE phosphoramidite was synthesized.

Before setting up the reaction, N⁶-Benzoyl-2′-deoxy-5′-O-DMT-7-deaza-2′-deoxyadenosine was dried in high vacuum (1 mbar, 0.1 kPa) overnight. CEP-Cl (215 µL, 0.91 mmol, 1.2 equiv.) under Argon at 0° C. was added to the stirred solution of N⁶-Benzoyl-2′-deoxy-5′-O-DMT-7-deaza-2′-deoxyadenosine (500 mg, 0.76 mmol) in dry dichloromethane (5 mL) and DIPEA (523 µL, 3.05 mmol, 4 equiv.). The cooling bath was removed after 10 minutes and the solution was stirred at ambient temperature for 3.5 hours. The solution was filtered through a syringe filter and diluted with 5 mL dichloromethane. The organic phase was washed with a saturated aqueous solution of NaHCO₃ (2x 10 mL) and brine (10 mL), then dried over anhydrous MgSO₄, filtered and concentrated in vacuo.

DMT = 4,4′-Dimethoxytrityl,

CEP-Cl = 2-Cyanoethyl-N,N-diisopropylchlorophosphoramidite Cl

DIPEA = N,N-Diisopropylethylamine

### Example 1b: Synthesis of N6-DMF-2′-deoxy-5′-O-DMT-7-deaza-8-aza-2′-deoxyadenosine

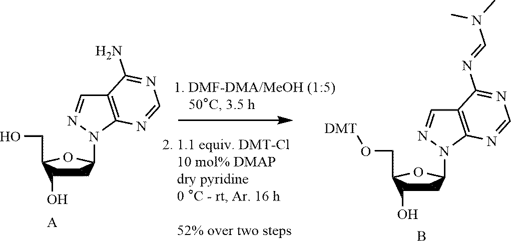

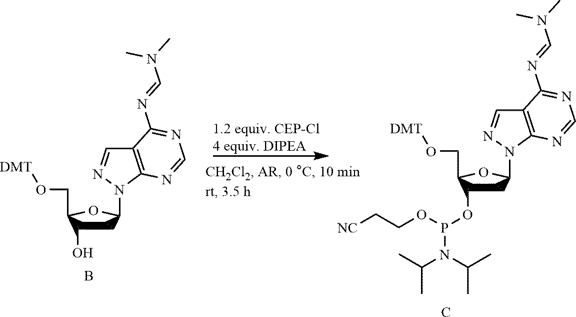

7-deazaaza-2′-deoxyadenosine was dried in high vacuum (1 mbar, 0.1 kPa) overnight before setting up the reaction.

Step 1: The solution of 7-deaza-8-aza-2′-deoxyadenosine A (500 mg, 2.00 mmol) in dry methanol (10 mL) and DMF-DMA (2 mL) was stirred at 50° C. for 3.5 hour. The solvents were removed under at 10 mbar (1 kPa). The orange residue was coevaporated twice with each 5 mL dry methanol and 5 mL diethyl ether, dried under vacuum, and then immediately used in Step 2.

Step 2: 4-(Dimethylamino)pyridin (DMAP;22.6 mg, 0.20 mmol, 10 mol%, in 0.4 mL dry pyridine) and DMT-Cl (752.1 mg, 2.20 mmol, 1.1 equiv., in 3.6 mL dry pyridine) at 0° C. under argon were added to the solution of N₆-DMF-2′-deoxy-7-deaza-8-azaadenosine (612 mg, 2.00 mmol) in dry pyridine (6 mL). The solution was stirred overnight allowing it to warm up to 25° C. The solvent was removed under reduced pressure and the residue was dissolved in 100 mL dichloromethane. The organic layer was washed with ice-cold brine (0° C., 3 x 50 mL) and ice-cold water (0° C., 1 x 50 mL), dried over MgSO₄ and removed under reduced pressure. The product B was purified by silica gel column chromatography using methanol/ dichloromethane as eluent.

### Synthesis of N⁶-DMF-2′-deoxy-5′-O-DMT-2′-7-deaza-8-aza-2′-deoxyadenosine 3′-CE phosphoramidite

N⁶-DMF-2′-deoxy-5′-O-DMT-2′-7-deaza-8-aza-2′-deoxyadenosine B was dried in high vacuum overnight before setting up the reaction. CEP-Cl (278 µL, 1.23 mmol, 1.2 equiv.) under Argon at 0° C. was added to the stirred solution of N⁶-DMF-2′-deoxy-5′-O-DMT-2′-7-deaza-8-aza-2′-deoxyadenosine (625 mg, 1.03 mmol) in dry dichloromethane (9 mL) and DIPEA (706 µL, 4.11 mmol, 4 equiv.). The cooling bath was removed after 10 minutes and the solution was stirred at ambient temperature for 3.5 hours. The solution was filtered through a stringe filter and diluted with 15 mL dichloromethane. The organic phase was washed with a saturated aqueous solution of NaHCO3 (2x 30 mL), brine (30 mL) then dried over anhydrous MgSO₄, filtered and concentrated in vacuo.

### Example 2: Synthesis of the 7-deaza-8-aza-guanine Building Block for Automated DNA Synthesis

Synthesis of N6-DMF-2′-deoxy-5′-O-DMT-7-deaza-8-aza-2′-deoxyguanosine

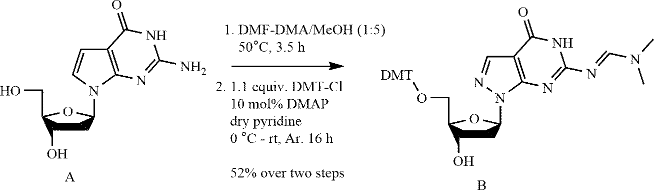

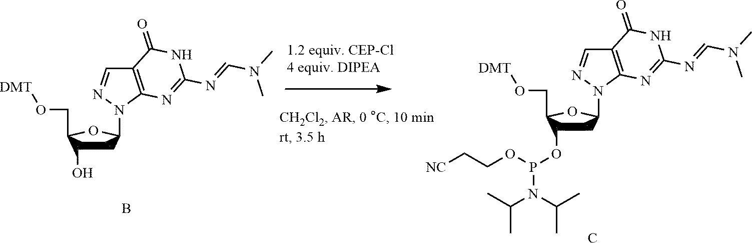

7-deazaaza-2′-deoxyguanosine was dried in high vacuum overnight was dried before setting up the reaction.

Step 1: The solution of 7-deaza-8-aza-2′-deoxyguanosine (500 mg, 2.00 mmol) in dry methanol (10 mL) and DMF-DMA (2 mL) was stirred at 50° C. for 3.5 hours. The solvents were removed under at 10 mbar (1 kPa). The orange residue was coevaporated twice with each 5 mL dry methanol and 5 mL diethyl ether, dried under vacuum, and then immediately used in the next step.

Step 2: DMAP (22.6 mg, 0.20 mmol, 10 mol%, in 0.4 mL dry pyridine) and DMT-Cl (752.1 mg, 2.20 mmol, 1.1 equiv., in 3.6 mL dry pyridine) at 0° C. under argon were added to the solution of N₆-DMF-2′-deoxy-7-deaza-8-azaguanosine (612 mg, 2.00 mmol) in dry pyridine (6 mL). The solution was stirred overnight allowing it to warm up to 25° C. The solvent was removed under at 10 mbar (1 kPa), and the residue was dissolved in 100 mL dichloromethane. The organic layer was washed with ice-cold brine (0° C., 3 x 50 mL) and ice-cold water (0° C., 1 x 50 mL), dried over MgSO₄ and removed under reduced pressure. The product was purified by silica gel column chromatography using methanol/ dichloromethane as eluent.

### Synthesis of N⁶-DMF-2′-deoxy-5′-O-DMT-2′-7-deaza-8-aza-2′-deoxyguanosine 3′-CE phosphoramidite

N⁶-DMF-2′-deoxy-5′-O-DMT-2′-7-deaza-8-aza-2′-deoxyguanosine was dried in high vacuum overnight before setting up the reaction. CEP-Cl (278 µL, 1.23 mmol, 1.2 equiv.) under Argon at 0° C. was added to the stirred solution of N⁶-DMF-2′-deoxy-5′-O-DMT-2′-7-deaza-8-aza-2′-deoxyguanosine (625 mg, 1.03 mmol) in dry dichloromethane (9 mL) and DIPEA (706 µL, 4.11 mmol, 4 equiv.). The cooling bath was removed after 10 minutes, and the solution was stirred at ambient temperature for 3.5 hours. The solution was filtered through a stringe filter and diluted with 15 mL dichloromethane. The organic phase was washed with a saturated aqueous solution of NaHCO3 (2x 30 mL) and brine (30 mL), then dried over anhydrous MgSO₄, filtered and concentrated in vacuo.

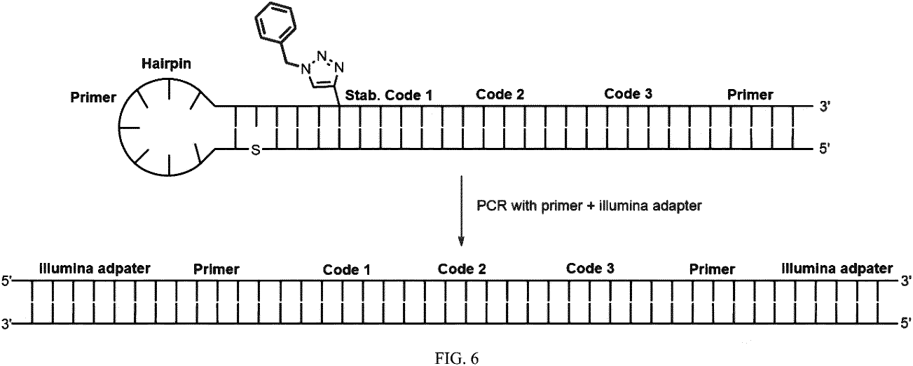

FIG. 5 illustrates the stabilized purines usable for automated DNA oligonucleotide synthesis and screening of the DNA oligonucleotides for stability. Examples 1a-1c and 2a-2b illustrate the synthesis of stabilized purines from readily available precursors, which obviates lengthy nucleoside synthesis; the stabilized purines can also be converted to phosphoramidites for automated DNA oligonucleotide synthesis in three, high-yielding steps without chromatography. Furthermore, the chemically modified phosphoramidites give comparable yields in DNA synthesis.

### Example 3: Chemical Stability of DNA Sequences

The chemical stability of DNA sequences was evaluated as previously described, experimental details are given below:

### Example 3a: Treatment of Solid Support-coupled DNA With Acids

20 nmol of controlled pore glass (CPG)-coupled oligonucleotide were treated with 50 µL of an acid according to Table 1 (e.g. 10% trifluoroacetic acid or 3.7% HCl). The suspension was shaken at ambient temperature for 22 h. Afterwards, the solution was removed by filtration, and the CPG was washed three times with each 200 µL of 0.1 M MgCl₂ solution, water, DMF, MeOH, CAN, and CH₂Cl₂ and dried in vacuo.

Cleavage and analysis: DNA was deprotected and cleaved from CPG by shaking the DNA in 500 µL of an AMA solution (AMA = aqueous ammonia (30%)/ aqueous methylamine (40%), 1:1, vol/vol) for 4 h at room temperature. Afterwards 20 µL of 1 M Tris buffer (pH = 7.5) were added, and the mixture was dried under at 10 mbar (1 kPa) (SpeedVac) and dissolved in 200 µL of distilled water. The product was analyzed by analytical RP-HPLC and MALDI-TOF-MS.

### Example 3b: Treatment of Solid Support-coupled DNA With Metal Salts

20 nmol of CPG-coupled oligonucleotide were treated with 200 equiv. of metal salt (4 µmol) dissolved in 50 µL dry solvent. The suspension was shaken at ambient temperature for 22 h. Afterwards, the solvent was removed by filtration, and the CPG was washed three times with each 200 µL of 0.1 M EDTA solution, 0.1 M MgCl₂ solution, water, DMF, MeOH, ACN and CH₂Cl₂ and dried in vacuo.

Cleavage and analysis: DNA was deprotected and cleaved from the CPG by shaking with 500 µL of an AMA solution (AMA = aqueous ammonia (30%)/ aqueous methylamine (40%), 1:1, vol/vol) for 4 h at room temperature. Afterwards, 20 µL of 1 M Tris buffer (pH = 7.5) were added, and the mixture was dried at 10 mbar (1 kPa) (SpeedVac), and the DNA was dissolved in 200 µL distilled water. The product was analyzed by Analytical RP-HPLC and MALDI-TOF-MS.

### Example 3c: Treatment of Solid Support-coupled DNA With Organocatalysts

20 nmol of CPG-coupled oligonucleotide (ca. 0.7 mg of solid phase) were treated with 200 equiv. of organocatalyst (4 µmol) dissolved in 50 µL dry solvent. The suspension was shaken at ambient temperature for 22 h. Afterwards, the solvent was removed by filtration, and the CPG was washed three times with each 0.1 M MgCl₂ solution, water, DMF, MeOH, ACN and CH₂Cl₂ and dried in vacuo.

Cleavage and analysis: DNA was deprotected and cleaved from CPG by shaking in 500 µL of an AMA solution (AMA = aqueous ammonia (30%)/ aqueous methylamine (40%), 1:1, vol/vol) for 4 h at room temperature. Afterwards, 20 µL of 1 M Tris buffer (pH = 7.5) were added, the mixture was dried at 10 mbar (1 kPa) (SpeedVac), and the DNA was dissolved in 200 µL distilled water. The product was analyzed by Analytical RP-HPLC and MALDI-TOF-MS.

TABLE 1

Stability of stabilized DNA oligonucleotides against aqueous acids, metal salts, and organic reagents^{a}. The percentages represent the amount of DNA degradation.

| Entry | Acid/metal salt/catalyst | Solvent | DNA oligonucleotides | | | | | |
|---|---|---|---|---|---|---|---|---|
| | | | ATC | ATGC | A*TC | A**TC | TG*C | A*TG*C |
| 1 | 10% TFA | | > 61% | > 61% | 0-20% | 0-20% | 0-20% | 21-40% |
| 2 | 3.7% HCl | | > 61% | > 61% | 0-20% | 0-20% | 41-60% | 41-60% |
| 3 | Bi(OTf)₃ | MeOH | 0-20% | 21-40% | 0-20% | 0-20% | --- | --- |
| 4 | Ce(NH₄)₂(NO₃)₆ | MeOH | > 61% | > 61% | 0-20% | 0-20% | 41-60% | 21-40% |
| 5 | Co(acac)₃ | ACN | 0-20% | 0-20% | 0-20% | 0-20% | 0-20% | 21-40% |
| 6 | Cu(MeCN)₄PF₆ | ACN | 21-40% | 21-40% | 0-20% | 0-20% | 0-20% | 0-20% |
| 7 | FeCl_{2●}4 H₂O | ACN | 0-20% | 0-20% | 0-20% | 0-20% | 0-20% | 0-20% |
| 8 | La(Oi-Pr)₃ | THF | 0-20% | 0- 20% | 0-20% | 0-20% | 0-20% | 0-20% |
| 9 | LiBr | ACN | 0-20% | 0-20% | 0-20% | 0-20% | --- | --- |
| 10 | Ni(acac)₂ | ACN | 0-20% | 0-20% | 0-20% | 0-20% | 0-20% | 0-20% |
| 11 | Pd(OAc)₂ | ACN | > 61% | > 61% | > 61% | > 61% | > 61% | > 61% |
| 12 | RuCl₃ | ACN | 41-60% | 21-40% | 21-40% | 21-40% | 21-40% | 21-40% |
| 13 | [Ru(p-cymene)Cl₂]₂ | CH₂Cl₂ | 21-40% | 41-60% | 21-40% | 0-20% | --- | --- |
| 14 | Grubbs 1^{st} Gen. | CH₂Cl₂ | 21-40% | 41-60% | 0-20% | 0-20% | 0-20% | 21-40% |
| 15 | Sc(OTf)₃ | ACN | 0-20% | 0-20% | 0-20% | 0-20% | 0-20% | 21-40% |
| 16 | Sc(OTf)₃, 40° C. | ACN | 41-60% | > 61% | 21-40% | 21-40% | 21-40% | 41-60% |
| 17 | SeO₂ | MeOH | 0-20% | 0-20% | 0-20% | 0-20% | 0-20% | 0-20% |
| 18 | VO(acac)₂ | MeOH | 0-20% | 0-20% | 0-20% | 0-20% | --- | --- |
| 19 | ZnCl₂ | ACN | 0-20% | 0-20% | 0-20% | 0-20% | 0-20% | 0-20% |
| 20 | DDQ | EtOH | 21-40% | 21-40% | 0-20% | 0-20% | 0-20% | 0-20% |
| 21 | PIDA | ACN | 41-60% | 41-60% | 0-20% | 0-20% | 0-20% | 21-40% |
| 22 | TEMPO | ACN | 0-20% | 20% | 0-20% | 0-20% | 0-20% | 0-20% |

^{a} for each: 20 nmol DNA, 200 eq. metal salt or organic reagent, 50 µL solvent, r.t., 22 h.
ACN = acetonitrile,
MeOH = methanol,
THF = tetrahydrofuran,
OTf = trifluoromethanesulfonate;
acac = acetylacetonate,
Grubbs 1^{st} Gen. = Benzylidenbis(tricyclohexylphosphin)dichlororuthenium,
DDQ = 2,3-dichloro-5,6-dicyanobenzoquinone,
PIDA = phenyliodine(III) diacetate,
TEMPO = 2,2,6,6-tetramethylpiperidinyloxyl
ATC = 5′-TTA CTA CCT A-3′-CPG (SEQ ID NO:1),
A*TC = 5′-TTA*CTA*CCA*T-3′-CPG (SEQ ID NO:2) with A* = 7-deaza-2′-deoxyadenosine,
A**TC = 5′-TTA**CTA**CCA**T-3′-CPG (SEQ ID NO:3) with A** = 7-deaza-8-aza-2′-deoxyadenosine;
ATGC = 5′-GTC ATG ATC T-3′-CPG (SEQ ID NO: 24);
TG*C = 5′-TTG* CTG* CCG* T-3′-CPG (SEQ ID NO: 25) with G* = 7-deaza-8-aza-2′-deoxyguanosine, A*TG*C = 5′-G*TC A*TG* A*TC T-3′-CPG (SEQ ID NO: 26) with A* = 7-deaza-2′-deoxyadenosine and G* = 7-deaza-8-aza-2′-deoxyguanosine.

A systematic screening of DNA stability in presence of aqueous acids, metal salts and organic reagents revealed that an ATC-sequence was strongly degraded in the presence of strong protic acids (see Table 1, entries 1-2), several metals (Table 1, entries 11-12) or metals with a high redox potential (see Table 1, entry 4). Higher temperature in combination with metals also led in several cases to high degrees of DNA degradation compared to the results under ambient temperature (Table 1, entries 15-16). Chemically stabilized DNA-strands containing 7-deaza-or 7-deaza-8-azaadenosine revealed a much higher stability under these conditions than the native DNA. For example, the treatment of the chemically modified DNA-strands with strong protic acids lead to no DNA-degradation (see Table 1, entries 1-2). Also in the presence of Ce(III) with a high redox potential, or the hypervalent iodine oxidant PIDA, the chemically modified DNA was completely stable (see Table 1, entries 4 and 21). Thus, the usage of 7-deaza- as well as 7-deaza-8-azaadenosine increased the stability of DNA. Consequently, the 8-aza-7-deaza-purines are compatible with established DNA-barcoding strategies relying on double-stranded DNA and offer the desired stability profile.

### Example 4: Exemplary Library Synthesis

Example 4a: Copper(I)-catalyzed alkyne-azide cycloaddition to install a linker moiety on chemically stabilized code I

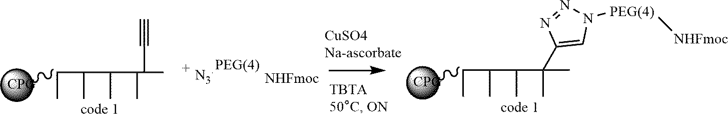

The CPG(controlled pore glass)-bound oligonucleotide (the oligonucleotide sequences, SEQ ID NO:27 and 28, used in this example and examples 4b, and 5-22 are provided in Table 2 below) alkyne conjugate (50 nmol) was suspended in 100 µL DMF and diluted with 380 µL of H₂O/MeOH (1:1). Subsequently, the azide (125 µmol, 2500 equiv.) was dissolved in 100 µL of DMF(9:1)/H₂O, Tris((1-benzyl-4-triazolyl)methyl)amine (TBTA) (6.25 µmol, 125 equiv.) dissolved in 20 µL of DMF, Na-ascorbate (6.25 µmol, 125 equiv.) dissolved in 10 µL of H₂O, and CuSO₄*5H₂O (0.625 µmol, 12.5 equiv.) dissolved in 10 µL of H₂O were added to the suspension in this order. Stock solutions of all reactants were prepared before the reaction was started. The reaction mixtures were shaken at 50° C. overnight. Then the CPG-bound conjugate was filtered over a filter column and washed three times with each 200 µL of 0.1 M EDTA solution, 0.1 M MgCl₂ solution, water, DMF, MeOH, ACN and CH₂Cl₂ and dried in vacuo.

The completeness of the reaction was controlled by cleavage of a small portion (~20 nmol) of CPG-bound oligonucleotide conjugate with 500 µL AMA (AMA = aqueous ammonia (30%)/ aqueous methylamine (40 %), 1:1, vol/vol) for 4 h at ambient temperature. To this solution 20 µL of 1 M Tris buffer (pH = 7.5) were added, the mixture was dried in a SpeedVac, re-dissolved in 200 µL of distilled water. The crude reaction product was analyzed by analytical RP-HPLC (Method II) and MALDI-MS.

Example 4b: Amide coupling to couple a bifunctional starting material to the code I

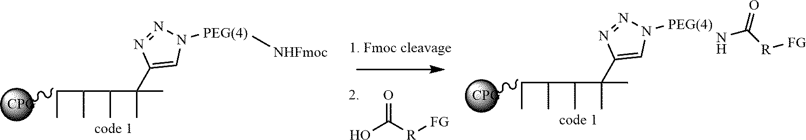

Protocol:
Step 1: The Fmoc-protecting group of the CPG-bound oligonucleotide (250 nmol) was cleaved off by addition of 200 µL 20 % piperidine in dry DMF and shaken for 5 min. Afterwards, the CPG-bound deprotected oligonucleotide was washed three times with each 200 µL of DMF, MeOH, ACN and CH₂Cl₂ and then dried in vacuo.
Step 2: CPG-coupled oligonucleotide, carboxylic acid (4-formyl-phenoxyacetic acid, N-Boc-tryptophan, N-Fmoc-piperidine-4-carboxylic acid, N-Fmoc-glycine, or 4-ethynylbenzoic acid) and HATU (O-(7-Azabenzotriazol-1-yl)-N,N,N′,N′-tetramethyluronium-hexafluorophosphate) were dried in vacuo for 15 min. Stock solutions of all reactants in dry DMF were prepared before the reaction was started. HATU (25 µmol, 100 equiv.) dissolved in 75 µL dry DMF and DIPEA (62.5 µmol, 250 equiv.) were added to the solution of carboxylic acid ((4-formyl-phenoxyacetic acid, N-Boc-tryptophan, N-Fmoc-piperidine-4-carboxylic acid, N-Fmoc-glycine, or 4-ethynylbenzoic acid) (25 µmol, 100 equiv.) in 75 µL dry DMF. The mixture was shaken for 5 min and added to CPG-coupled DNA suspended in 75 µL dry DMF (250 nmol, 1 equiv.). The amide coupling reaction was shaken at ambient temperature for 2 hours. Next, the CPG-coupled conjugate was filtered over a filter column, washed three times with each 200 µL of DMF, MeOH, ACN and CH₂Cl₂ and dried in vacuo. Amide coupling was repeated two times.

Completion of amide coupling was checked by cleaving off a 5% of CPG-coupled oligonucleotide conjugate (0.7-0.9 mg, ~20 nmol) with 500 µL AMA (AMA = aqueous ammonia (30%)/ aqueous methylamine (40%), 1:1, vol/vol) for 30 min (hexT) or 4 h (ATGC-sequences) at ambient temperature. Afterwards, 20 µL of 1 M Tris buffer (pH = 7.5) were added, the mixture was dried under reduced pressure (SpeedVac) and DNA was dissolved in 200 µL distilled water. The crude reaction mixture was analyzed by analytical RP-HPLC and MALDI-MS. In the case of uncompleted coupling (<90%) the reaction was repeated a third time.

Unreacted amines were capped with acetic acid anhydride (three times 200 µL, 30 s, 1:1 mixture of THF/methylimidazole, 9:1, vol/vol, and THF/pyridine/acetic acid anhydride 8:1:1, vol/vol). The capped CPG-coupled oligonucleotide conjugate was washed three times with each 200 µL of DMF, MeOH, ACN and CH₂Cl₂ and dried in vacuo.

### Example 5: Barcoded Compound Synthesis by Ugi Four-Component Reaction

Ugi four-component reaction on CPG-coupled code I

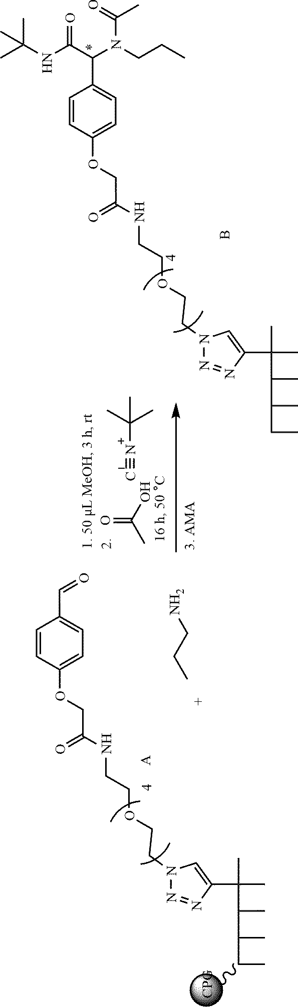

### Protocol

Prior to use, CPG-bound oligonucleotide aldehyde conjugate A was dried in vacuo for 15 min. A solution of propargylamine (1000 equiv., 20 µmol) in 50 µL MeOH was added to the CPG-bound DNA-aldehyde conjugate A. The reaction mixture was shaken at ambient temperature for 3 h to effect imine formation. Afterwards, acetic acid (1000 equiv., 20 µmol, solid acids were dissolved in 15 µL MeOH) was pipetted to the reaction mixture, followed by the addition of tert-butyl isocyanide (1000 equiv., 20 µmol). The reaction mixture was shaken for 16 h at 50° C. The CPG-bound conjugate B was filtered over a filter column, washed three times with each 200 µL of DMF, MeOH, ACN and CH₂Cl₂ and dried in vacuo. The CPG-bound DNA conjugate B was cleaved from the solid phase and deprotected with 500 µL AMA solution for 4 h at ambient temperature. Afterwards the mixture was dried in a SpeedVac and the remaining DNA pellet was dissolved in 200 µL of distilled water. The crude was analyzed by analytical RP-HPLC and MALDI-MS. The product was isolated by preparative RP-HPLC.

### Example 6: Barcoded Compound Synthesis by Ugi-azide Four-Component Reaction

Ugi-azide four-component reaction on CPG-coupled code I

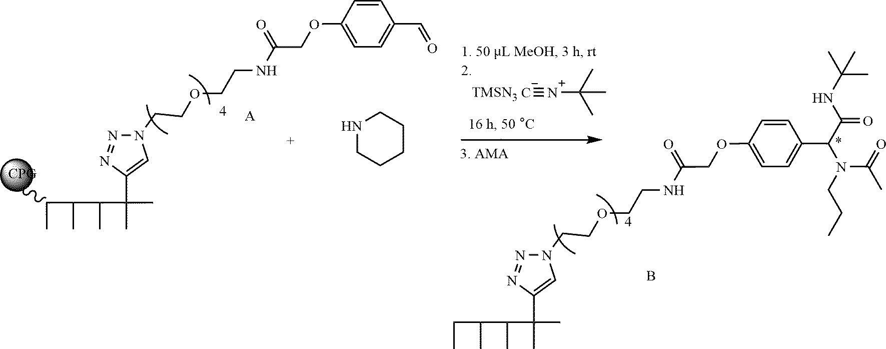

### Protocol

CPG-bound oligonucleotide A was dried in vacuo for 15 min. A solution of piperidine (1000 equiv., 20 µmol) in 50 µL MeOH was added to the CPG-bound DNA-aldehyde conjugate A. The reaction mixture was shaken at ambient temperature for 3 h to effect imine formation. Afterwards, tert-butylisocyanide (1000 equiv., 20 µmol) was pipetted to the reaction mixture, followed by the addition of azidotrimethylsilane (1000 equiv., 20 µmol). The reaction mixture was shaken for 16 h at 50° C. The CPG-bound conjugate B was filtered over a filter column, washed three times with each 200 µL of DMF, MeOH, ACN and CH₂Cl₂ and dried in vacuo. The CPG-bound DNA conjugate B was cleaved from the solid phase and deprotected with 500 µL AMA solution for 4 h at ambient temperature. Afterwards the mixture was dried in a SpeedVac and the remaining DNA pellet was dissolved in 200 µL of distilled water. The crude was analyzed by analytical RP-HPLC and MALDI-MS. The product was isolated by preparative RP-HPLC.

### Example 7: Barcoded Compound Synthesis by by Acid-Mediated Groebke-Blackburn-Bienaymé Three-Component Reaction

Acetic acid-mediated Groebke-Blackburn-Bienayme three-component reaction on CPG-coupled code I

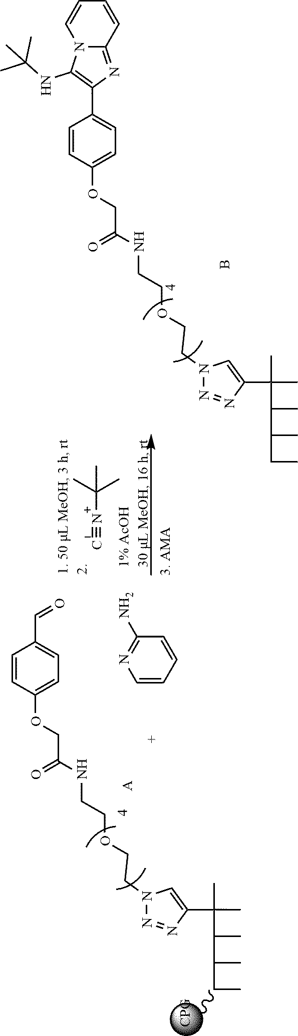

Protocol: Prior to use, CPG-bound oligonucleotide aldehyde conjugate A and 2-aminopyridine were dried in vacuo for 15 min. 2-aminopyridine (1000 equiv., 20 µmol) was added to the CPG-bound DNA-aldehyde conjugate A in 50 µL MeOH. The reaction mixture was shaken at ambient temperature for 6 h to effect imine formation. Afterwards, tert-butylisocyanide 16 (1000 equiv., 20 µmol) was pipetted to the reaction mixture, followed by the addition of acetic acid as Brønsted acid (final volume: 80 µL, acid concentration: 1%). The reaction mixture was shaken for 16 h at ambient temperature. The CPG-bound conjugate B was filtered over a filter column, washed three times with each 200 µL of DMF, MeOH, ACN and CH₂Cl₂ and dried in vacuo. The CPG-bound DNA conjugate B was cleaved from the solid phase and deprotected with 500 µL AMA solution for 4 h at ambient temperature. Afterwards the mixture was dried in a SpeedVac and the remaining DNA pellet was dissolved in 200 µL of distilled water. The crude was analyzed by analytical RP-HPLC and MALDI-MS. The product was isolated by preparative RP-HPLC.

### Example 8: Barcoded Compound Synthesis by Ugi Four-Component/Aza-Wittig Reaction

Ugi four-component/aza-Wittig reaction on CPG-coupled code I

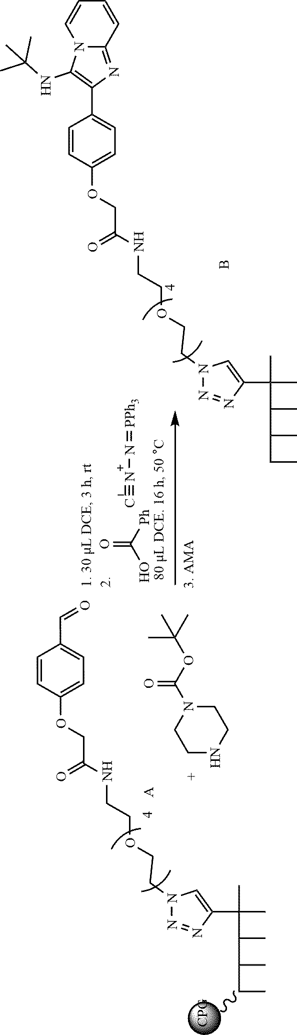

### Protocol

CPG-bound oligonucleotide A, N-Boc-piperazine, (isocyanoimino)triphenylphosphorane and solid acids were dried in vacuo for 15 min. N-Boc-piperazine (1000 equiv., 20 µmol) was added to the CPG-bound DNA-aldehyde conjugate A in 30 µL 1,2-dichloroethane. The reaction mixture was shaken at ambient temperature for 3 h to effect imine formation. Then, the acid (1000 equiv., 20 µmol) was dissolved in 80 µL 1,2-dichloroethane, transferred to (isocyanoimino)triphenylphosphorane (1000 equiv., 20 µmol) and this mixture was added to the CPG-bound conjugate A. The reaction mixture was shaken for 16 h at 50° C. The CPG-bound conjugate B was filtered over a filter column, washed three times with each 200 µL of DMF, MeOH, ACN and CH₂Cl₂ and dried in vacuo. The CPG-bound DNA conjugate B was cleaved from the solid phase and deprotected with 500 µL AMA solution for 4 h at ambient temperature. Afterwards the mixture was dried in a SpeedVac and the remaining DNA pellet was dissolved in 200 µL of distilled water. The crude was analyzed by analytical RP-HPLC and MALDI-MS. The product was isolated by preparative RP-HPLC.

### Example 9: Barcoded Compound Synthesis by Acid-Mediated Biginelli Reaction

(R)-(-)-BNDHP-mediated Biginelli reaction on CPG-coupled code I

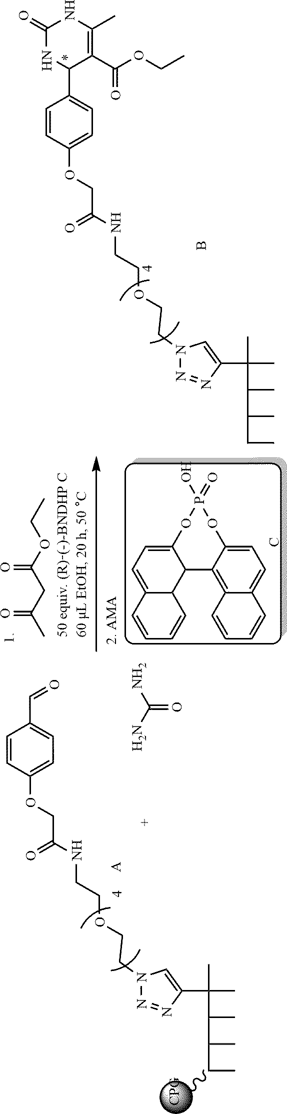

### Protocol

The CPG-bound oligonucleotide A, urea and (R)-(-)-BNDHP C were dried in vacuo for 15 min. Urea (10 µmol, 500 equiv.) and (R)-(-)-BNDHP C (1 µmol, 50 equiv.) were dissolved both in 30 µL ethanol. The solutions were added to CPG-coupled oligonucleotide-aldehyde conjugate A (20 nmol) followed by ethyl acetoacetate (10 µmol, 500 equiv.). The reaction mixture was shaken at 50° C. for 20 h. Then the CPG-bound oligonucleotide conjugate B was filtered over a filter column, washed three times with each DMF, MeOH, ACN and CH2Cl2 and dried in vacuo. CPG-bound oligonucleotide conjugates B were cleaved from solid support and deprotected with 500 µL AMA at ambient temperature for 4 h. Afterwards 20 µL of 1 M Tris buffer (pH = 7.5) were added, the mixture was dried under reduced pressure (SpeedVac) and DNA was dissolved in 200 µL distilled water. The crude reaction mixture was analyzed by analytical RP-HPLC and MALDI-TOF-MS. The product was purified by preparative RP-HPLC.

### Example 10: Barcoded Compound Synthesis by Acid-Mediated Povarov Reaction

(R)-(-)-BNDHP-mediated Povarov reaction on CPG-coupled code I

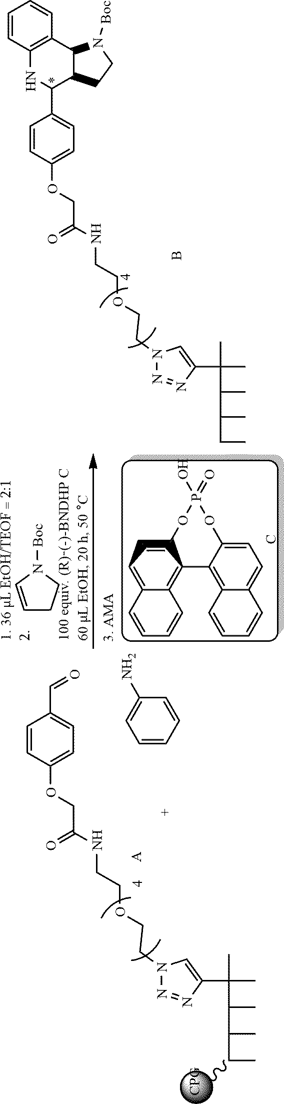

### Protocol

Prior to use, CPG-bound oligonucleotide A, solid anilines and (R)-(-)-BNDHP C were dried in vacuo for 15 min. Aniline (10 µmol, 500 equiv.) was dissolved in 24 µL ethanol. The solution was added to CPG-bound oligonucleotide-aldehyde conjugate A (20 nmol) suspended in 12 µL triethyl orthoformate. The suspension was shaken at ambient temperature for 4 h. Afterwards 30 µL of (R)-(-)-BNDHP C (2 µmol, 100 equiv.) in ethanol followed by N-Boc-2,3-dihydro-1H-pyrrole 30 (10 µmol, 500 equiv.) was added. The reaction mixture was shaken at 50° C. for 16 h. Then the CPG-bound oligonucleotide conjugate B was filtered over a filter column, washed three times with each DMF, MeOH, ACN and CH₂Cl₂ and dried in vacuo. CPG-bound oligonucleotide conjugates B were cleaved from solid support and deprotected with 500 µL AMA at ambient temperature for 4 h. Afterwards 20 µL of 1 M Tris buffer (pH = 7.5) were added, the mixture was dried under reduced pressure (SpeedVac) and DNA was dissolved in 200 µL distilled water. The crude reaction mixture was analyzed by analytical RP-HPLC and MALDI-TOF-MS. The product was purified by preparative RP-HPLC.

### Example 11: Barcoded Compound Synthesis by Acid-mediated Povarov Reaction

(R)-(-)-BNDHP-mediated Povarov reaction and following Boc-cleavage on CPG-coupled code I

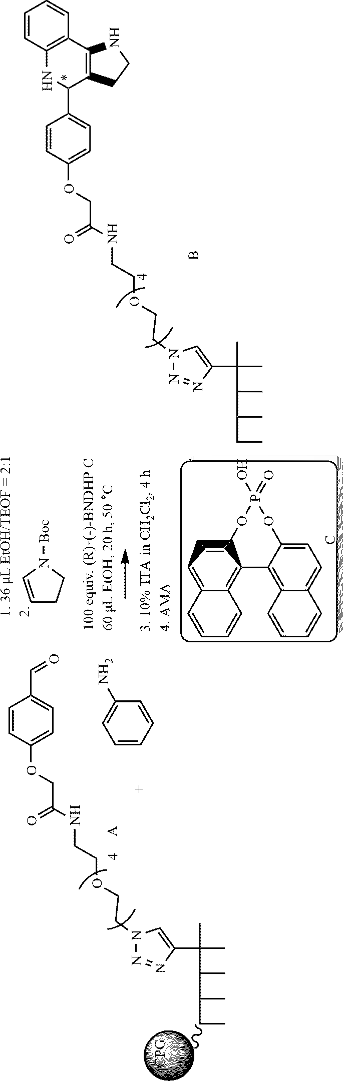

### Protocol

Prior to use, CPG-coupled oligonucleotide, solid anilines, and (R)-(-)-BNDHP (1,1′-Binaphthyl-2,2′-diyl hydrogenphosphate) were dried in vacuo for 15 min. Aniline (10 µmol, 500 equiv.) was dissolved in 24 µL ethanol. The solution was added to CPG-coupled oligonucleotide-aldehyde conjugate A (20 nmol) suspended in 12 µL triethyl orthoformate. The suspension was shaken at ambient temperature for 4 h. Afterwards, 30 µL of (R)-(-)-BNDHP C (2 µmol, 100 equiv.) in ethanol followed by N-Boc-2,3-dihydro-1H-pyrrole (10 µmol, 500 equiv.) were added. The reaction mixture was shaken at 50° C. for 16 h. Then, the CPG-coupled oligonucleotide conjugate was filtered over a filter column, washed three times with each DMF, MeOH, CAN, and CH₂Cl₂ and dried in vacuo. The Boc-protecting group was removed by addition of 100 µL 10% trifluoroacetic acid in CH₂Cl₂ for 4 h. Afterwards, CPG-coupled DNA was filtered over a filter column, washed with excess of 1% TEA and three times with each 200 µL of DMF, MeOH, ACN and CH₂Cl₂ and dried in vacuo. CPG-coupled oligonucleotide conjugates B were cleaved from solid support and deprotected with 500 µL AMA (AMA = aqueous ammonia (30%)/ aqueous methylamine (40%), 1:1, vol/vol) for 4 hours at ambient temperature. Afterwards, 20 µL of 1 M Tris buffer (pH = 7.5) were added, the mixture was dried under reduced pressure (SpeedVac) and DNA was dissolved in 200 µL distilled water. The crude reaction mixture was analyzed by analytical RP-HPLC (Method I) and MALDI-TOF-MS. The product was purified by preparative RP-HPLC.

### Example 12: Barcoded Compound Synthesis by Acid-Mediated Pictet-Spengler Reaction

TFA-mediated Pictet-Spengler reaction on CPG-coupled code I

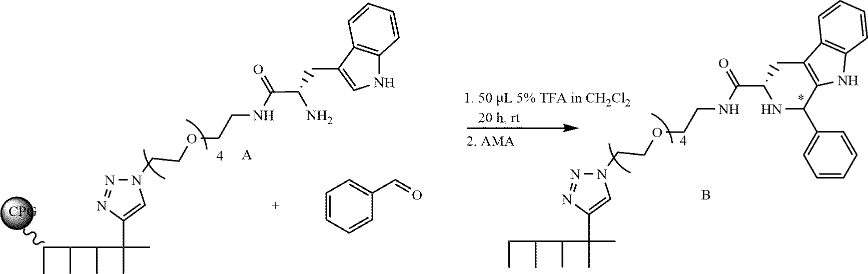

### Protocol

Prior to use, CPG-bound oligonucleotide A and the aldehyde (for solids) were dried in vacuo for 15 min. Aldehyde (30 µmol, 1500 equiv.) was dissolved in 50 µL of a 5% trifluoroacetic acid in CH₂Cl₂ solution. This solution was added to CPG-bound oligonucleotide-tryptophan conjugate A (20 nmol) and the reaction mixture was shaken at ambient temperature for 20 h. Afterwards CPG bound DNA was filtered over a filter column, washed with excess of 1% TEA and three times with each 200 µL of DMF, MeOH, ACN and CH₂Cl₂ and dried in vacuo. CPG-bound oligonucleotide conjugates B were cleaved from solid support and deprotected with 500 µL AMA at ambient temperature for 4 h. Afterwards 20 µL of 1 M Tris buffer (pH = 7.5) were added, the mixture was dried under reduced pressure (SpeedVac) and DNA was dissolved in 200 µL distilled water. The crude reaction mixture was analyzed by analytical RP-HPLC and MALDI-TOF-MS. The product was purified by preparative RP-HPLC.

### Example 13: Barcoded Compound Synthesis by Lewis Acid-Mediated Aza-Diels-Alder Reaction

Zn(II)-mediated aza-Diels-Alder reaction on CPG-coupled code I

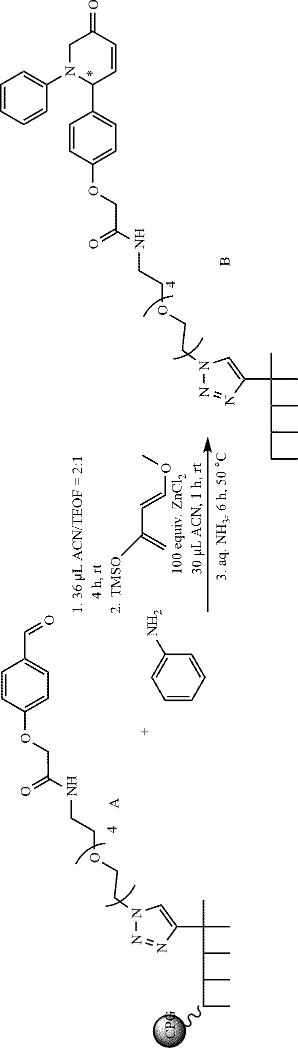

### Protocol

CPG-bound oligonucleotide A, and ZnCl₂ were dried in vacuo for 15 min. Aniline (10 µmol, 500 equiv.) was dissolved in 24 µL acetonitrile. The solution was added to CPG-coupled oligonucleotide-aldehyde conjugate A (20 nmol) suspended in 12 µL triethyl orthoformate. The suspension was shaken at ambient temperature for 4 h. Afterwards 30 µL of ZnCl₂ (2 µmol, 100 equiv.) in ACN followed by Danisheskys’s diene (20 µmol, 1000 equiv.) was added. The reaction mixture was shaken for 1 h at ambient temperature. Then the CPG-coupled oligonucleotide conjugate B was filtered over a filter column, washed three times with each 200 µL of 0.1 M EDTA solution, 0.1 M MgCl₂ solution, water, DMF, MeOH, ACN and CH₂Cl₂ and dried in vacuo. CPG-coupled oligonucleotide conjugate B was cleaved from solid support and deprotected with 200 µL aqueous ammonia (30%) at 50° C. for 6 h. Afterwards 20 µL of 1 M Tris buffer (pH = 7.5) were added, the mixture was dried under reduced pressure (SpeedVac) and DNA was dissolved in 200 µL distilled water. The crude reaction mixture was analyzed by analytical RP-HPLC and MALDI-TOF-MS. The product was purified by preparative RP-HPLC.

### Example 14: Barcoded Compound Synthesis by Lewis Acid-Mediated Petasis Reaction

Cu(I)/bpy-mediated Petasis reaction on CPG-coupled code I

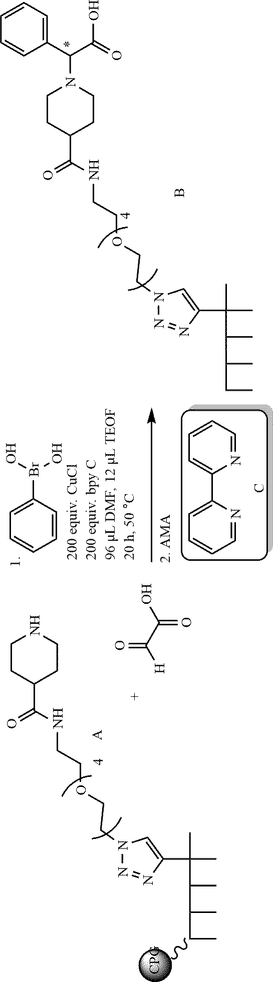

### Protocol

Prior to use all solid materials were dried in vacuo for 30 min. CuCl (4.0 µmol, 200 equiv., 40 mM calculated for the final volume of 100 µL) and 2,2′-bipyridine (bpy C, 4.0 µmol, 200 equiv., 40 mM calculated for the final volume of 100 µL) were dissolved in 48 µL DMF. The solution was shaken at 50° C. for 1 h. Phenylboronic acid (50 µmol, 2500 equiv., 500 mM calculated for the final volume of 100 µL) were dissolved in the CuCl/bpy C solution in DMF. 12 µL triethyl orthoformate and glyoxylic acid (40 µmol, 2000 equiv., 400 mM calculated for the final volume of 100 µL) dissolved in 40 µL DMF were added. The solution was added to CPG-coupled-DNA-secondary amine conjugate A (20 nmol, 1 equiv.) and the suspension was shaken at ambient temperature for 24 h. Then the CPG-bound DNA conjugate B was filtered over a filter column, washed three times with each 200 µL of 0.1 M EDTA solution, 0.1 M MgCl₂ solution, water, DMF, MeOH, ACN and CH₂Cl₂ and dried in vacuo. CPG-bound oligonucleotide conjugated α-aryl glycine B were cleaved from solid support and deprotected with 500 µL AMA for 4 h at ambient temperature. Afterwards 20 µL of 1 M Tris buffer (pH = 7.5) were added, the mixture was dried under reduced pressure (SpeedVac) and the DNA was dissolved in 200 µL distilled water. The crude reaction mixture was analyzed by analytical RP-HPLC and MALDI-TOF-MS. The product was purified by preparative RP-HPLC.

### Example 15: Barcoded Compound Synthesis by Lewis Acid-Mediated 1,3-dipolar Cycloaddition Reaction

Ag(I)-mediated 1,3-dipolar cycloaddition reaction on CPG-coupled code I

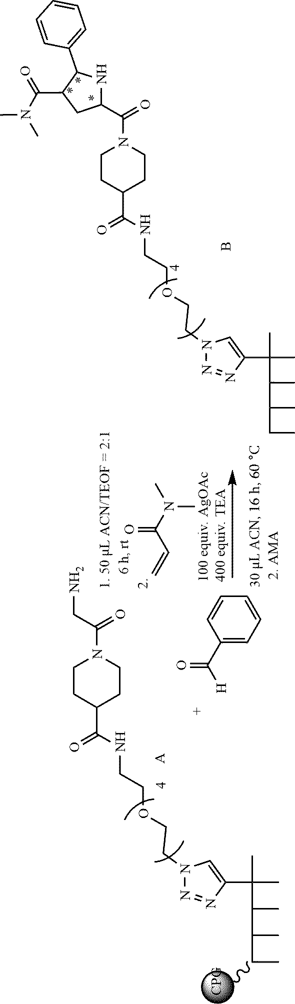

### Protocol

CPG-bound oligonucleotide A was dried in vacuo for 15 min. Benzaldehyde (1000 equiv., 20 µmol) was added to the CPG-bound DNA-glycine conjugate A in 50 µL ACN/triethyl orthoformate (2:1). The reaction mixture was shaken at ambient temperature for 6 h. Afterwards, 30 µL of a suspension of AgOAc (100 equiv., 2 µmol) in ACN/triethyl orthoformate (2:1) was added followed by N,N-dimethylacrylamide (4000 equiv., 80 µmol) and TEA (4000 equiv., 80 µmol). Prior addition to the reaction vessel, the AgOAc suspension was vortexed and pipetted up and down to obtain a homogeneous suspension. The reaction mixture was shaken for 16 h at 50° C. Afterwards CPG bound DNA B was filtered over a filter column, washed with excess of 1% TEA and three times with each 200 µL of 0.1 M EDTA solution, 0.1 M MgCl₂ solution, water, DMF, MeOH, ACN and CH₂Cl₂ and dried in vacuo. CPG-bound oligonucleotide conjugates B were cleaved from solid support and deprotected with 500 µL AMA at ambient temperature for 4 h. Afterwards 20 µL of 1 M Tris buffer (pH = 7.5) were added, the mixture was dried under reduced pressure (SpeedVac) and DNA was dissolved in 45 µL distilled water. 5 µL of 1,3,5-triazine-2,4,6-trithiol trisodium salt solution (15% in H₂O) were added and the solution was shaken for 30 min at ambient temperature. Afterwards the sample was centrifuged at 4° C. for 30 min (13200 rpm; Centrifuge 5415 R, Eppendorf), the supernatant was taken off and diluted with 5 µL of a 3 M sodium acetate (pH = 5.2) and 200 µL 100% ethanol. The solution was incubated overnight at -80° C. Afterwards the samples were centrifuged at 4° C. for 30 min (13200 rpm; Centrifuge 5415R, Eppendorf), the supernatant was taken off, additional 100 µL of 100% ethanol were added to the pellet and the solution was incubated again for 1 h at -80° C. Afterwards the sample was centrifuged at 4° C. for 30 min (13200 rpm; Centrifuge 5415R, Eppendorf), the supernatant was taken off, and the DNA pellets were dried at 37° C. The DNA samples were dissolved in 100 µL ddH₂O. The crude was analyzed by analytical RP-HPLC and MALDI-MS. The product was purified by preparative RP-HPLC.

### Example 16: Barcoded Compound Synthesis by Lewis Acid-Mediated Castagnoli-Cushman Reaction

Yb(III)-mediated Castagnoli-Cushman reaction on CPG-coupled code I

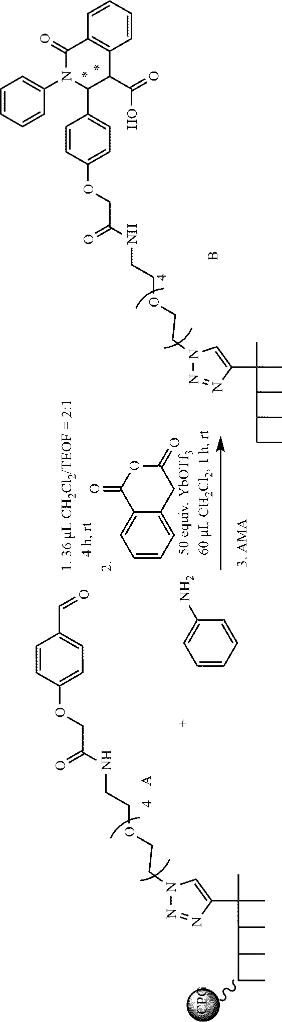

### Protocol

CPG-bound oligonucleotide A, homophtalic anhydride and Yb(OTf)₃ were dried in vacuo for 15 min. Aniline (10 µmol, 500 equiv.) was dissolved in 24 µL CH₂Cl₂. The solution was added to the CPG-bound oligonucleotide-aldehyde conjugate A (20 nmol) suspended in 12 µL triethyl orthoformate. The suspension was shaken at ambient temperature for 4 h. Afterwards 30 µL of a suspension of Yb(OTf)₃ (1 µmol, 50 equiv.) in CH₂Cl₂ was added, followed by 30 µL of a suspension of homophtalic anhydride 45 (10 µmol, 500 equiv.) in CH₂Cl₂. Prior addition to the reaction vessel both suspensions were vortexed and pipetted up and down to obtain homogeneous suspensions. The reaction mixture was shaken for 1 h at ambient temperature. Then the CPG-bound conjugate B was filtered over a filter column and washed three times with each 200 µL of 0.1 M EDTA solution, 0.1 M MgCl₂ solution, water, DMF, MeOH, ACN and CH₂Cl₂ and dried in vacuo. CPG-bound oligonucleotide conjugate Bwas then cleaved from the solid support and deprotected with 500 µL AMA solution for 4 h at ambient temperature. To this solution 20 µL of 1 M Tris buffer (pH = 7.5) were added, the mixture was dried in a SpeedVac and afterwards dissolved in 200 µL of distilled water. The crude was analyzed by analytical RP-HPLC and MALDI-MS. The product was purified by preparative RP-HPLC.

### Example 17: Barcoded Pyrazole Synthesis by Lewis Acid-Mediated Three-Component Reaction

Yb(III)-mediated three-component reaction for pyrazole synthesis on CPG-coupled code I

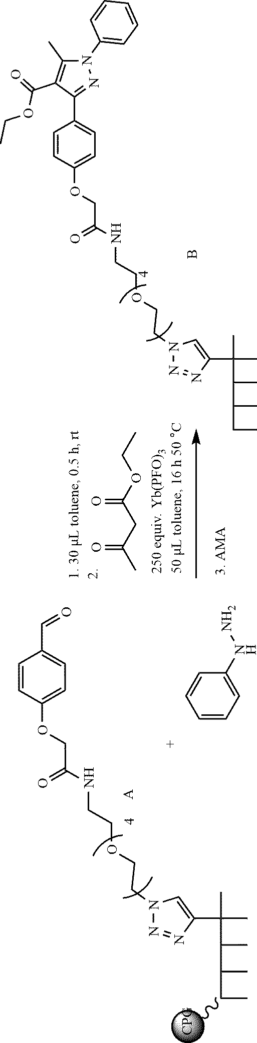

### Protocol

The catalyst Yb(PFO)₃ (PFO=perfluorooctanoate) was prepared according to a published procedure (L. Wang, J. Han, J. Sheng, H. Tian, Z. Fan, Catal. Commun. 2005, 5, 201-204.). Prior to the reaction, the hydrazine was extracted with diluted NH₃ solution and CH₂Cl₂, dried over MgSO₄ and finally dried in vacuo if the hydrazine was present as a hydrochloride salt. The hydrazine (250 equiv., 5 µmol), dissolved in 30 µL toluene was added to the CPG-bound DNA-aldehyde conjugate A and the reaction mixture was shaken at ambient temperature for 0.5 h. Afterwards, ethyl acetoacetate (3000 equiv., 60 µmol) and 50 µL of a suspension of Yb(PFO)₃ (250 equiv., 5 µmol) in toluene was added. Prior addition to the reaction vessel the Yb(PFO)₃ suspension was vortexed and pipetted up and down to obtain a homogeneous suspension. The reaction mixture was shaken at 50° C. for 16 h. The CPG-bound conjugate B was filtered over a filter column and washed with each 3x 200 µL of 0.1 M EDTA solution, 0.1 M MgCl₂ solution, water, DMF, MeOH, ACN and CH₂Cl₂ and then dried in vacuo. The CPG-bound DNA conjugate B was cleaved from the solid phase and deprotected by adding 500 µL AMA solution and shaking for 4 hours at ambient temperature. Afterwards the mixture was dried in a SpeedVac and the remaining DNA pellet was dissolved in 200 µL of distilled water.

### Example 18: Barcoded Pyrazoline-Containing Spiroheterocycle Synthesis by Lewis Acid-Mediated Three-Component Reaction

Au(I)/Ag(I)-promoted pyrazoline-containing spiroheterocycle synthesis on CPG-coupled code I

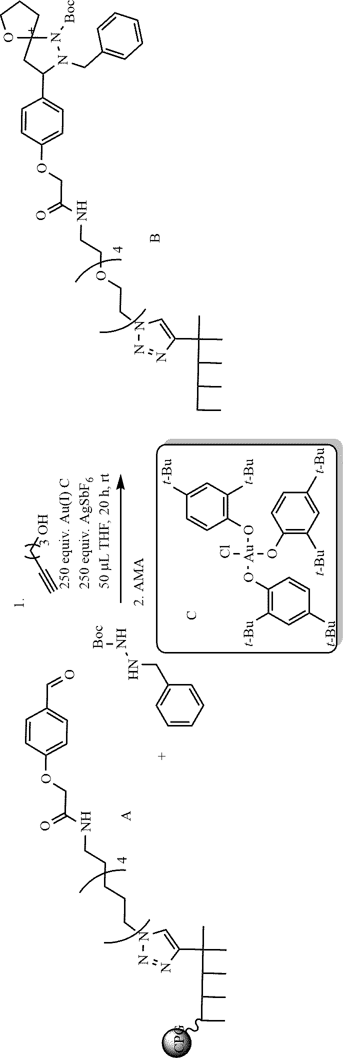

### Protocol

CPG-bound oligonucleotide A, tert-butyl 2-benzylhydrazinecarboxylate, [Tris(2,4-di-tert-butylphenyl)phosphite]gold chloride C and AgSbF₆ were dried in vacuo for 15 min. The solution of tert-butyl 2-benzylhydrazine-carboxylate (500 equiv., 15 µmol) in 20 µL THF and pent-4-yn-1-ol (1000 equiv., 30 µmol) were added to CPG- bound DNA-aldehyde conjugate A (30 nmol) followed by equimolar mixture of Au(I)/AgSbF₆ (250 equiv., 7.5 µmol) suspended in 30 µL THF. Prior addition to the reaction vessel the mixture was vortexed and pipetted up and down. The reaction mixture was shaken at room temperature for 20 h. Then the CPG-bound conjugate B was filtered over a filter column and washed three times with each 200 µL of 0.1 M EDTA solution, 0.1 M MgCl₂ solution, water, DMF, MeOH, ACN and CH₂Cl₂ and dried in vacuo. CPG-bound oligonucleotide conjugate B was then cleaved from the solid support and deprotected with 500 µL AMA solution for 4 h at ambient temperature. To this solution 20 µL of 1 M Tris buffer (pH = 7.5) were added, the mixture was dried in a SpeedVac and afterwards dissolved in 45 µL of distilled water. 5 µL of 1,3,5-triazine-2,4,6-trithiol trisodium salt solution (15% in H₂O) were added and the solution was shaken for 30 min at ambient temperature. Afterwards the sample was centrifuged at 4° C. for 30 min (13200 rpm; Centrifuge 5415 R, Eppendorf), the supernatant was taken off and diluted with 5 µL of a 3 M sodium acetate (pH = 5.2) and 200 µL 100% ethanol. The solution was incubated overnight at -80° C. Afterwards the samples were centrifuged at 4° C. for 30 min (13200 rpm; Centrifuge 5415 R, Eppendorf), the supernatant was taken off, additional 100 µL of 100% ethanol were added to the pellet and the solution was incubated again for 1 h at -80° C. Afterwards the sample was centrifuged at 4° C. for 30 min (13200 rpm; Centrifuge 5415 R, Eppendorf), the supernatant was taken off, and the DNA pellets were dried at 37° C. The DNA samples were dissolved in 100 µL ddH₂O. The crude was analyzed by analytical RP-HPLC and MALDI-MS. The product was purified by preparative RP-HPLC.

### Example 19: Barcoded Pyrazoline Synthesis by Lewis Acid-Mediated Three-Component Reaction

Au(I)/Ag(I)-promoted pyrazoline synthesis on CPG-coupled code I

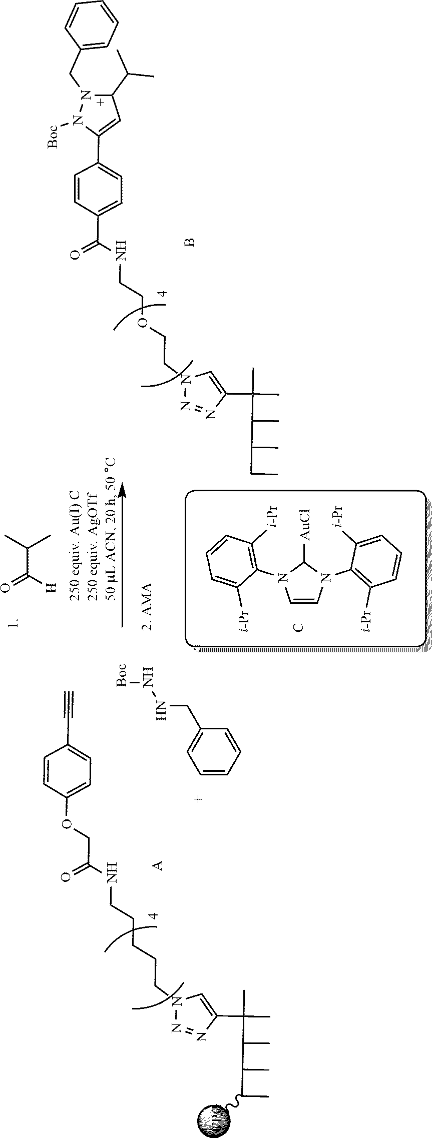

### Protocol

CPG-bound oligonucleotide A, tert-butyl 2-benzylhydrazinecarboxylate, chloro[1,3-bis(2,6-diisopropylphenyl)imidazol-2-ylidene] gold(I) C and AgOTf were dried in vacuo for 15 min. The solution of tert-butyl 2-benzylhydrazine-carboxylate (1000 equiv., 20 µmol) in 20 µL dry acetonitrile and aliphatic aldehyde (1000 equiv., 20 µmol) were added to CPG- bound DNA-alkyne conjugate A (20 nmol) followed by equimolar mixture of Au(I)/AgOTf (250 equiv., 5 µmol) suspended in dry 30 µL acetonitrile. Prior addition to the reaction vessel the mixture was vortexed and pipetted up and down. The reaction mixture was shaken at room temperature for 20 h. Then the CPG-bound conjugate B was filtered over a filter column and washed three times with each 200 µL of 0.1 M EDTA solution, 0.1 M MgCl₂ solution, water, DMF, MeOH, ACN and CH₂Cl₂ and dried in vacuo. CPG-bound oligonucleotide conjugate B was then cleaved from the solid support and deprotected with 500 µL AMA solution for 4 h at 50° C. To this solution 20 µL of 1 M Tris buffer (pH = 7.5) were added, the mixture was dried in a SpeedVac and afterwards dissolved in 45 µL of distilled water. 5 µL of 1,3,5-triazine-2,4,6-trithiol trisodium salt solution (15% in H₂O) were added and the solution was shaken for 30 min at ambient temperature. Afterwards the sample was centrifuged at 4° C. for 30 min (13200 rpm; Centrifuge 5415 R, Eppendorf), the supernatant was taken off and diluted with 5 µL of a 3 M sodium acetate (pH = 5.2) and 200 µL 100% ethanol. The solution was incubated overnight at -80° C. Afterwards the samples were centrifuged at 4° C. for 30 min (13200 rpm; Centrifuge 5415 R, Eppendorf), the supernatant was taken off, additional 100 µL of 100% ethanol were added to the pellet and the solution was incubated again for 1 h at -80° C. Afterwards the sample was centrifuged at 4° C. for 30 min (13200 rpm; Centrifuge 5415 R, Eppendorf), the supernatant was taken off, and the DNA pellets were dried at 37° C. The DNA samples were dissolved in 100 µL ddH₂O. The crude was analyzed by analytical RP-HPLC and MALDI-MS. The product was purified by preparative RP-HPLC.

### Example 20: Au(I)/Ag(I)-Promoted Pyrazole Synthesis on DNA Barcode

Au(I)/Ag(I)-promoted pyrazole synthesis on CPG-coupled code I

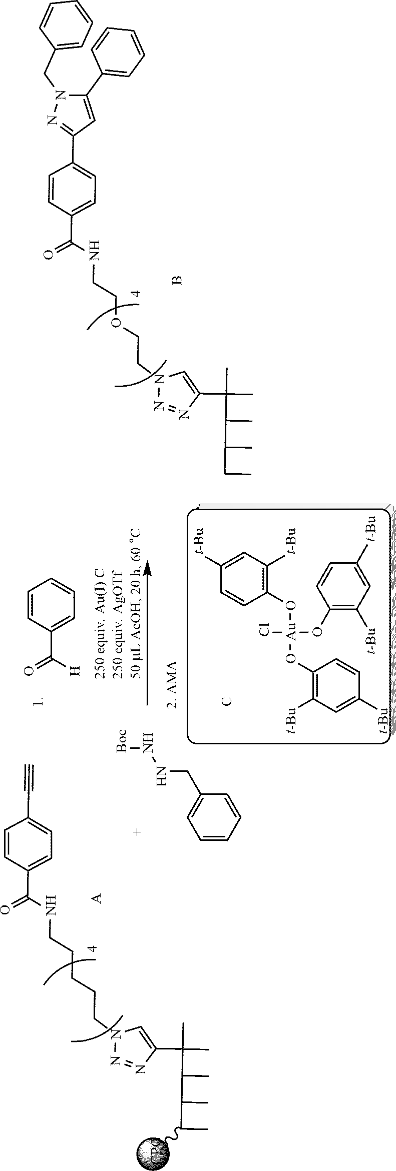

### Protocol

CPG-bound oligonucleotide A, tert-butyl 2-benzylhydrazinecarboxylate, [tris(2,4-di-tert-butylphenyl)phosphite]gold chloride C and AgOTf were dried in vacuo for 15 min. The solution of tert-butyl 2-benzylhydrazine-carboxylate (1000 equiv., 20 µmol) in 20 µL glacial acetic acid and aldehyde (1000 equiv., 20 µmol) were added to CPG- bound DNA-alkyne conjugate A (20 nmol) followed by equimolar mixture of Au(I)/AgOTf (250 equiv., 5 µmol) suspended in dry 30 µL glacial acetic acid. Prior addition to the reaction vessel the mixture was vortexed and pipetted up and down. The reaction mixture was shaken at 60° C. for 20 h. Then the CPG-bound conjugate was filtered over a filter column and washed three times with each 200 µL of 0.1 M EDTA solution, 0.1 M MgCl₂ solution, water, DMF, MeOH, ACN and CH₂Cl₂ and dried in vacuo. CPG-bound oligonucleotide conjugate B was then cleaved from the solid support and deprotected with 500 µL AMA solution for 4 h at ambient temperature. To this solution 20 µL of 1 M Tris buffer (pH = 7.5) were added, the mixture was dried in a SpeedVac and afterwards dissolved in 45 µL of distilled water. 5 µL of 1,3,5-triazine-2,4,6-trithiol trisodium salt solution (15% in H₂O) were added and the solution was shaken for 30 min at ambient temperature. Afterwards the sample was centrifuged at 4° C. for 30 min (13200 rpm; Centrifuge 5415 R, Eppendorf), the supernatant was taken off and diluted with 5 µL of a 3 M sodium acetate (pH = 5.2) and 200 µL 100% ethanol. The solution was incubated overnight at -80° C. Afterwards the samples were centrifuged at 4° C. for 30 min (13200 rpm; Centrifuge 5415 R, Eppendorf), the supernatant was taken off, additional 100 µL of 100% ethanol were added to the pellet and the solution was incubated again for 1 h at -80° C. Afterwards the sample was centrifuged at 4° C. for 30 min (13200 rpm; Centrifuge 5415 R, Eppendorf), the supernatant was taken off, and the DNA pellets were dried at 37° C. The DNA samples were dissolved in 100 µL ddH₂O. The crude was analyzed by analytical RP-HPLC and MALDI-MS. The product was purified by preparative RP-HPLC.

### Example 21: Cu(II)-Mediated Tin-Amine Protocol (SnAP) Reaction on DNA Barcode

Cu(II)-promoted piperazine synthesis on CPG-coupled code I

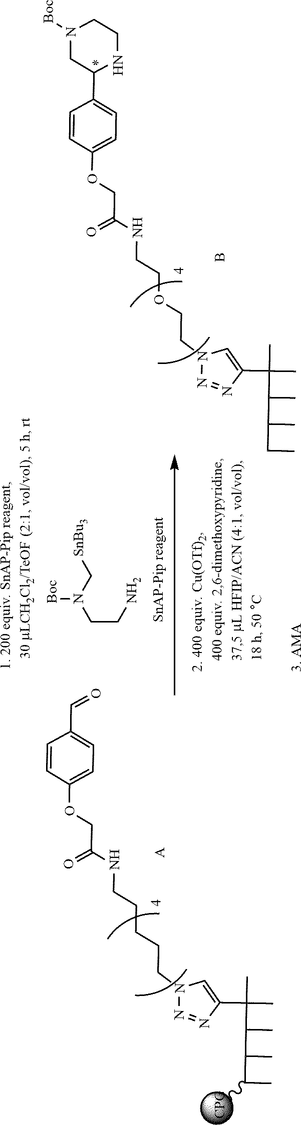

### Protocol

Prior to use, commercially available Cu(OTf)₂ (Strem) was dried under high vacuum at 110° C. for overnight. Pre-dried Cu(OTf)₂ (400 equiv., 12 µmol) was dissolved in 22.5 µL of a 2:1 mixture HFIP/ACN (vol/vol) followed by addition of 2,6-dimethoxypyridine (400 equiv., 12 µmol). The dark greenish mixture was shaken at room temperature for 5 h. Simultaneously, SnAP-Pip reagent (200 equiv., 6 µmol) was added to a suspension of 30 nmol CPG-bound DNA-aldehyde conjugate A in 30 µL of a 2:1 mixture CH₂Cl₂/TEOF (vol/vol). The reaction mixture was shaken at room temperature for 5 h. After that, 15 µL HFIP were added to the suspension of crude CPG-bound imine, followed by activated Cu(OTf)₂ solution. The reaction mixture was shaken at 50° C. for 18 h. After that, CPG-bound DNA-piperazine conjugate B was filtered over a filter column, washed three times with each 200 µL of 0.1 M EDTA, 0.1 M MgCl₂, DMF, MeOH, ACN and CH₂Cl₂ and dried in vacuo. CPG-bound oligonucleotide conjugate B was cleaved from the solid support with 500 µL AMA (AMA = aqueous ammonia (30%)/ aqueous methylamine (40%), 1:1, vol/vol) for 4 h at room temperature. Cleavage was quenched by addition of 20 µL of 1 M Tris buffer (pH = 7.5). The solvent was removed under reduced pressure (SpeedVac) and remaining DNA-conjugate was re-dissolved in 200 µL of distilled water. After filtration, the crude product was analyzed by analytical RP-HPLC and MALDI-MS.

### Example 22: Acid-Mediated Boc-Cleavage in Aqueous Solution

TFA-mediated Boc-cleavage on code I in aqueous solution

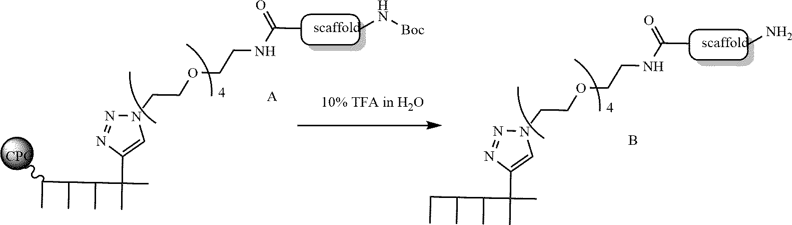

### Protocol

An isolated pellet of Boc-protected oligonucleotide conjugate A was dissolved in 20 µL of a 10% trifluoroacetic acid in H₂O solution. The solution was shaken at ambient temperature for 4 h. The Boc deprotected oligonucleotide conjugate was precipitated by adding 2 µL of a 3 M sodium acetate (pH = 5.2) and 80 µL of 100% ethanol and storing this solution for overnight at -80° C. Afterwards, the samples were centrifuged at 4° C. for 30 min (13200 rpm; Centrifuge 5415 R, Eppendorf), the supernatant was taken off and the DNA pellets were dried. Oligonucleotide conjugate was dissolved in ddH₂O and analyzed by analytical RP-HPLC and MALDI-TOF-MS

### Example 23: Degradation of CPG-bound Stabilized DNA Barcodes

TABLE 2

Overview of diverse chemical reactions on CPG-bound stabilized barcodes

| Entry | Reaction | Conversion^{a} (Degradation^{b}) | |
|---|---|---|---|
| | | A*TC | A*TG*C |
| 1 | Ugi four-component reaction (Example 5) | 92 (<5) | 91 (<5) |
| 2 | Ugi-azide four-component reaction (Example 6) | >95 (6) | >95 (<5) |
| 3 | Groebke-Blackbum-Bienayme reaction (Example 7) | >95 (<5) | 84^{c} (<5) |
| 4 | Ugi/aza-Wittig reaction (Example 8) | 39 (24) | 53 (44) |
| 5 | Biginelli reaction (Example 9) | 87 (<5) | 86 (<5) |
| 6 | Povarov reaction (Example 10) | >95 (<5) | 79^{d} (<5) |
| 7 | Boc cleavage (Example 11) | >95 (<5) | --- |
| 8 | Pictet-Spengler reaction (Example 12) | 93 (<5) | 87 (29) |
| 9 | aza-Diels-Alder reaction (Example 13) | 71 (<5) | 68 (<5) |
| 10 | Petasis reaction (Example 14) | 90 (<5) | 69 (<5) |
| 11 | 1,3-Dipolar cycloaddition (Example 15) | 49 (<5) | --- |
| 12 | Castagnoli-Cushman reaction (Example 16) | 56 (<5) | 43 (<5) |
| 13 | Three-component pyrazoles synthesis (Example 17) | 50 (33) | 17 (31) |
| 14 | Pyrazoline-spiroheterocycle synthesis (Example 18) | 69 (<5) | 59^{e} (<5) |
| 15 | Pyrazoline synthesis (Example 19) | 85 (<5) | 85 (<5) |
| 16 | Pyrazole synthesis (Example 20) | 88 (8) | 65^{f} (24) |
| 17 | Piperazine synthesis (Example 21) | --- | 55 (<5) |
| 18 | TFA-mediated Boc-cleavage in aqueous solution (Example 22) | >95 (<5) | >95 (~90) |

^{a,b} Conversion and DNA degradation determined by HPLC. ^{c} additional 11% of undefined byproduct.
^{d} additional 12% of undefined byproduct. ^{e} additional 15% of undefined byproducts. ^{f} additional 20% of undefined byproducts.
A^{∗}TC = 5′-CT*CTCTTTA*A*CTA*CCT-3 (SEQ ID NO:27);
A^{∗}TG^{∗}C = 5′-CT*CTCG*A*TTCG*CA**CCT-3′ (SEQ ID NO:28);
T^{∗}= attachment point for the respective linker/organic moiety
A^{∗} = 7-deaza-2′-deoxyadenosine;
G^{∗} = 7-deaza-8-aza-2′-deoxyguanosine;
A^{∗∗} = 7-deaza-8-aza-2′-deoxyadenosine;
n.d. = not detected.

### Example 23: DNA Amplification and Sequencing Experiments

Gel electrophoresis of two PCR experiments performed on chemically modified DNA templates. The templates were synthesized by DNA ligation of DNA barcode fragments, and the PCR was performed with primers used to read out DEL selection experiments. PCR amplification of templates consisting of (1) 8-aza-7-deaza-2′-deoxyadenosine (5′-CT^{∗}(PEG4-NH-Ac)CTCTTTA^{∗∗}A^{∗∗}CTA^{∗∗}CCT-3′ (SEQ ID NO:29); A^{∗∗}= 7-deaza-8-aza-2′-deoxyadenosine; T^{∗} = ethynyl-dU was modified by copper-mediated alkyne-azide cycloaddition with N₃-PEG4-NH-Fmoc followed by Fmoc cleavage and simple acetylation of the free amine to yield dU-triazole-PEG4-NH-Ac), and (2) 7-deazaadenosine (5′-CT^{∗}CTCTTTA^{∗}A^{∗}CTA^{∗}CCT-3′ (SEQ ID NO:27); A^{∗} = 7-deazaadenosine; T^{∗} = dU-triazole-PEG4-NH-Ac) by Taq polymerase were compared head-to-head. PCR amplification products obtained from a template containing three 7-deaza-adenines and a template containing three 8-aza-7-deazaadenines were obtained at similar efficiencies. Even more important, a Sanger sequencing run proved high fidelity copying of both modified DNA strands. This result shows that DNA modified with 8-aza-7-deazaadenosines can be copied by PCR with standard nucleotide triphosphates. 8-aza-7-deazaadenosine positions at isolated positions or two in a row were read with the same fidelity as 7-deazaadenosine (data not shown). The experiment shown in FIG. 4 was successfully repeated, i.e. similar results were obtained albeit not shown in FIG. 4, with a template containing two 8-aza-7-deazaguanosines and a single 8-aza-7-deazaadenosine.

A standard PCR amplification reaction can be performed on a template that contains 8-aza-7-deazapurines. 8-aza-7-deazapurines appear as fully functional, chemically stable substitutes in DNA barcodes and show the same chemical stability as pyrimidine bases, and are read by Taq polymerase. 8-aza-7-deazapurines also show the same chemical stability as pyrimidine bases.

### Example 24: Design of a Barcoded Compound and a PCR Amplicon

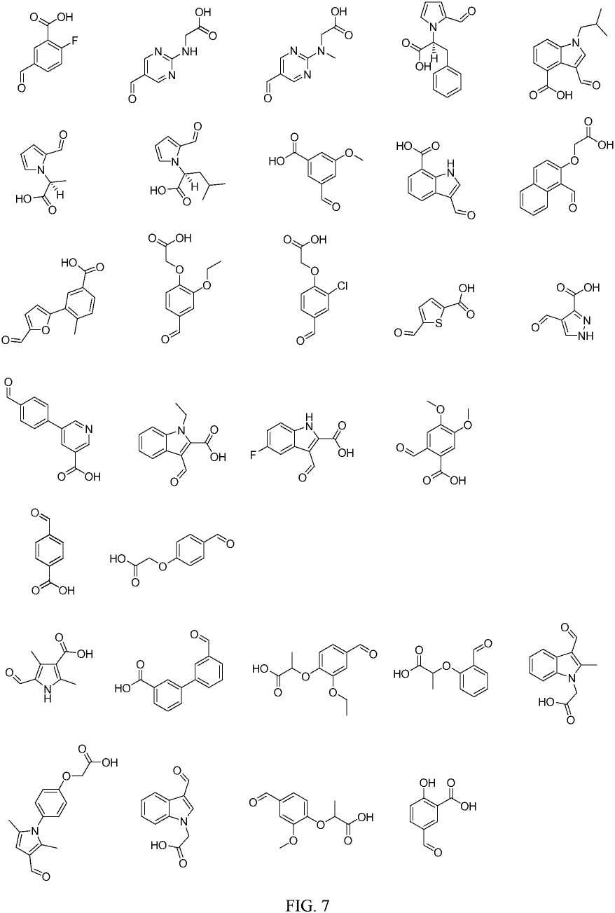

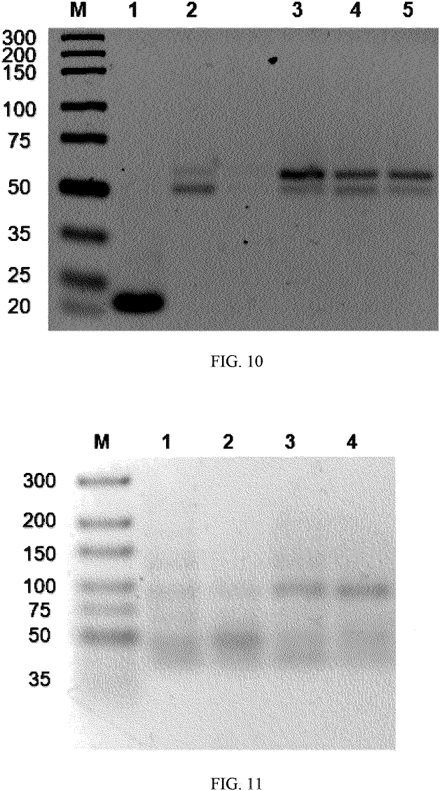

FIG. 6 shows an embodiment of a design of a final DNA-tagged compound and a PCR amplicon thereof. A DNA barcode included a linker within the sequence as opposed to at the 5′-terminal position of the sequence. Said differently, the linker was shifted from the 5′-terminal to the 5′ penultimate position as described herein. FIG. 6 illustrates the encoding strategy using this position of the linker. Bifunctional starting materials that contain a carboxylic acid and a carbaldehyde (representative examples see FIG. 8) were coupled to Code 1 through their carboxylic acid moiety, then pooled and reacted as pools to target compounds by a synthesis method discussed in Example 11 for a barcoded compound synthesis by acid-mediated Povarov reaction, above. The final compounds were cleaved from the solid phase, quality controlled, and ligated to a DNA hairpin containing the forward primer and a code 2 recording the heterocycle synthesis (FIG. 3). Alternatively, the encoded library was finalized by a DNA-compatible coupling reaction and a further DNA ligation step according to FIG. 4. The encoding strategy by ligation of chemically modified DNA barcode 1 (sequence II) to hairpin sequence I and barcode 2 was successfully validated.

This encoding strategy provides chemical diversity, can be scaled up, and provides fidelity when the more demanding chemistry, such as several heterocycle formation reactions, is performed earlier in the process. This encoding strategy provides flexibility, as e.g. DNA hairpin and codes 2 and 3 could also be composed of stable nucleobases if the chemistry done in the last step demands stable nucleobases.

### Example 25: Coding of a Two-cycle DNA-Encoded Library

Hairpin DNA, coding oligonucleotides and ligation products were phosphorylated at the 5′-end with T4 polynucleotide kinase (T4 PNK, Thermo Scientific Scientific). The phosphorylation reactions were performed in a total volume of 20 µL using 280 pmol of the oligonucleotides, 10 units of T4 polynucleotide kinase (T4 PNK, Thermo Fisher Scientific), 1x PNK Buffer A (500 mM Tris-HCl, 100 mM MgCh, 50 mM DTT, 1 mM spermidine, pH = 7.6, 25° C., Thermo Fisher Scientific) and 1 mM ATP (Thermo Fisher Scientific) were used. Reaction mixtures were incubated at 37° C. for 20 min, then heat-inactivated at 75° C. for 15 min and slowly cooled down to 4° C.

Prior to enzymatic ligation of DNA, the oligonucleotides were annealed by incubation at 85° C. for 10 min and cooling down to 4° C. For ligation (20 µL scale), 100 pmol of each oligonucleotide, 600 units of T4 DNA Ligase (T4 DNA ligase rapid, Biozym) and 1x T4 DNA Ligase Buffer (500 mM Tris-HCl, 100 mM MgCl₂, 50 mM DTT, 10 mM ATP, pH = 7.6 at 25° C.) were mixed. Ligation reactions were performed at 25° C. for 16 h, then stopped by heat inactivation at 75° C. for 15 min and cooled down to 4° C.

For analysis of DNA ligation reactions, agarose gel electrophoresis was performed. Using a 5.5% agarose gel, electrophoresis was carried out in TBE buffer (89 mM Tris-borate, 2 mM EDTA, pH = 8.3) at 100 V constant voltage for 15 min and then 150 V constant voltage for about 45 min. For staining of the DNA, Midori Green Direct (NIPPON Genetics) and as a reference, GeneRuler Ultra Low Range DNA Ladder (Thermo Fisher Scientific) was used. Imaging of the gels was performed using the Bio-Rad Gel Doc™ XR system.

Table 3 shows the functionality and the DNA sequences of the different elements, which were used in the ligation process for a two-cycle library (FIGS. 2 and 3). Table 4 shows the functionality and the DNA sequences, which were used in the ligation process for a three-cycle library (FIGS. 2 and 3).

TABLE 3

Functions of the partial sequences of oligonucleotides I, II, II′, III and III′ (FIG. 2 )

| | | | |
|---|---|---|---|
| partial sequence | function | length | sequence (5′-3′) |
| A | forward primer | 21 mer | AGG TCG GTG TGA ACG GATTTG |
| B | overhang for T4 DNA ligation | 4 mer | AGTC |
| S | spacer (Taq DNA polymerase blocker) | - | C₉ spacer |
| B′ | overhang for T4 DNA ligation | 4 mer | GACT |
| C | chemically modified DNA code (code 1) | 8 mer | XXXXXXXX |
| | | | composed of C, T, A*, A**, G* for example: |
| | | | CTT TA*(A**, G*)A*(A**, G*) CT |
| C′ | Universal sequence to chemically modified DNA code (code 1) | 8 mer | i aai iaa i |
| D | overhang for T4 DNA ligation | 4 mer | CCTA |
| D′ | overhang for T4 DNA ligation | 4 mer | TAGG |
| E | code 2 | 8 mer | XXX XXX XX |
| | | | X composed of C, T, A, G |
| E′ | complementary sequence to code 2 | 8 mer | XXX XXX XX |
| | | | X composed of C, T, A, G |
| F | reverse primer | 23 mer | TGAC CTCA ACTA CATG GTCT ACA (SEO ID NO:10) |
| F″ | complementary sequence to reverse | 23 mer | TGTA GACC ATGT AGTT GAGG TCA (SEO ID NO: 11) |

A* denotes 7-deaza adenosine; A** denotes 7-deaza-8-aza adenosine; G* denotes 7-deaza-8-aza guanosine; i denotes inosine; a denotes abasic site.

TABLE 4

Sequences of exemplary oligonucleotides I, II, II′, III, and III′ (FIG. 3 ).

| | | |
|---|---|---|
| DNA | length | sequence (5′-3′) |
| I | 37 mer | CAA ATC CGT TCA S AGG TCG GTG TGA ACG GAT TTG AGTC |
| II | 16 mer | CT*C TCT TTA* A*CT A*CC T (code A) (SEQ ID NO:12) CT*C TCT TTA** A**CT A**CC T (code B) (SEQ ID NO:13) CT*C TCT TTG* G*CT A**CC T (code C) (SEQ ID NO:14) |
| II′ | 24 mer | TAG GAG GTi aai iaa iAG AGG ACT (SEQ ID NO:6) |
| III | 35 mer | CCT AGA CTT GAC TGA CCT CAA CTA CAT GGT CTA CA (SEO ID NO:7) |
| III′ | 31 mer | TGT AGA CCA TGT AGT TGA GGT CAG TCA AGT C (SEQ ID NO:8) |

A^{∗} denotes 7-deaza adenosine; A^{∗∗} denotes 7-deaza-8-aza adenosine; G^{∗} denotes 7-deaza-8-aza guanosine; i denotes inosine; a denotes abasic site; T^{∗} denotes T that serves as an attachment point to the linker/organic molecule; for validation of the encoding strategy in this example benzyl was attached via triazole to a dU nucleotide by copper-mediated alkyne-azide cycloaddition of in situ formed benzyl azide to 5-ethynyl-dU. T^{∗} is thus U with a triazole-benzyl moiety in the 5-position

### Example 25: PCR Amplification of Chemically Modified Nucleic Acids

Ligation reaction of the hairpin DNA I with the chemically modified duplex DNA II/II′ and native duplex DNA III/III′ (as set forth in Table 4). The following reactions were carried out and analyzed by gel electrophoresis: 1: one-pot ligation of hairpin DNA I with DNA duplex II/II′ and DNA duplex III/III′; 2: ligation of hairpin DNA I with DNA duplex II/II′, 7-deaza-8-aza adenosine was used. The results show that in all reactions carried out the ligation product was obtained (data not shown).

DNA obtained by enzymatic ligation of hairpin DNA I to chemically modified duplex DNA II/II′ and duplex DNA III/III′ (FIGS. 2-3, Tables 3-4) were amplified by PCR (forward primer (54mer): 5′-TCG TCG GCA GCG TCA GAT GTG TAT AAG AGA CAG AGG TCG GTG TGA ACG GAT TTG-3′ (SEQ ID NO:15), reverse primer (57mer): 5′-GTC TCG TGG GCT CGG AGA TGT GTA TAA GAG ACA GTG TAG ACC ATG TAG TTG AGG TCA-3′ (SEQ ID NO: 16).

For PCR amplification (40 µL scale), 10 µL of ligation product, 1 µM of reverse primer, 5 units of Taq DNA polymerase (Thermo Fisher Scientific), 1x Taq DNA Polymerase Buffer (100 mM Tris-HCl, pH 8.8 at 25° C., 500 mM KCl, 0.8% (v/v) Nonidet P40), 3 mM MgCl₂ and 0.625 mM of each dNTP (dATP, dGTP, dTTP, and dCTP, corresponding to 2.5 mM of the mixture of dNTPs) were mixed.

The PCR program started with pre-denaturation at 95° C. for 3 min, followed by denaturation for 30 s at 95° C., annealing for 30 s at 55° C., and elongation for 30 s at 72° C. After 10 PCR cycles, 1 µM of forward primer were added and additional 20 PCR cycles were performed.

For analysis of DNA amplification reactions, agarose gel electrophoresis was performed. Using a 5.5% agarose gel, electrophoresis was carried out in TBE buffer (89 mM Tris-borate, 2 mM EDTA, pH = 8.3) at 100 V constant voltage for 15 min and then 150 V constant voltage for about 45 min. For staining of the DNA, Midori Green Direct (NIPPON Genetics) and as a reference, GeneRuler Ultra Low Range DNA Ladder (Thermo Fisher Scientific) was used. Imaging of the gels was performed using the Bio-Rad Gel Doc™ XR system.

Gel analysis of a PCR amplification of chemically modified DNA ligation product. 1: PCR product of DNA ligation product containing DNA hairpin I, DNA duplex II/II′ and DNA duplex III/III′; 2: one-pot ligation of hairpin DNA I with DNA duplex II/II′ and DNA duplex III/III′, 3: ligation of hairpin DNA I with DNA duplex II/II′; 7-deaza adenosine was used. The results show that in all reactions carried out the ligation product was obtained (data not shown).

PCR amplification of chemically modified DNA ligation product. 1: DNA hairpin I; 2: one-pot ligation of hairpin DNA I with DNA duplex II/II′ and DNA duplex III/III′; 3: PCR product of DNA ligation product containing DNA hairpin I, DNA duplex II/II′ and DNA duplex III/III′. 7-deaza-8-aza adenosine was used. The results show that in all reactions carried out the ligation product was obtained (data not shown).

### Example 26: Sequencing of Chemically Modified Nucleic Acids

DNA obtained by PCR amplification were purified using the QIAquick PCR Purification Kit (QIAGEN) according to the protocol. Sequencing of the DNA was done by GATC.

The sequencing data was aligned against the template DNA synthesized from chemically modified code A, code B or code C as shown in Table 4. The data showed the expected sequence (data not shown).

### Example 27: Synthesis of a Three-Cycle Library

Hairpin DNA, coding oligonucleotides and ligation products were phosphorylated at the 5′-end with T4 polynucleotide kinase (T4 PNK, Thermo Fisher Scientific). The phosphorylation reactions were performed in a total volume of 20 µL using 280 pmol of the oligonucleotides, 10 units of T4 polynucleotide kinase (T4 PNK, Thermo Fisher Scientific), 1x PNK Buffer A (50 mM Tris-HCl, 10 mM MgCl₂, 5 mM DTT, 0.1 mM spermidine, pH = 7.6, 25° C., Thermo Fisher Scientific) and 1 mM ATP (Thermo Fisher Scientific) were used. Reaction mixtures were incubated at 37° C. for 20 min, then heat-inactivated at 75° C. for 15 min and slowly cooled down to 4° C.

Prior to enzymatic ligation of DNA, the oligonucleotides were annealed by incubation at 85° C. for 10 min and cooling down to 4° C. For ligation (20 µL or 40 µL scale), 100 pmol of each oligonucleotide, 600 units of T4 DNA Ligase (T4 DNA ligase, New England Biolabs)) and 1x T4 DNA Ligase Buffer (50 mM Tris-HCl, 10 mM MgCl₂, 10 mM DTT, 1 mM ATP, pH = 7.5 at 25° C., New England Biolabs) were mixed. Ligation reactions were performed at 25° C. for 16 h, then stopped by heat inactivation at 75° C. for 15 min and cooled down to 4° C.

For analysis of DNA ligation reactions, agarose gel electrophoresis was performed. Using a 3 or 4% agarose gel, electrophoresis was carried out in TBE buffer (89 mM Tris-borate, 2 mM EDTA, pH = 8.3) at 150 V constant voltage for about 45 min. For staining of the DNA, Midori Green Direct (NIPPON Genetics) and as a reference, GeneRuler Ultra Low Range DNA Ladder (Thermo Fisher Scientific) was used. Imaging of the gels was performed using the Bio-Rad Gel Doc™ XR system.

After the first and second ligation, the DNA was precipitated by adding ⅒ volume of 3 M aq. sodium acetate (pH = 5.2) and 3 volumes of 100% ethanol and incubating this solution for about 4 h or overnight at -80° C. Afterwards the samples were centrifuged at 4° C. for 30 min (13200 rpm; Centrifuge 5415 R, Eppendorf), the supernatant was taken off, additional 100 µL of 100% ethanol were added and the solution was incubated for 1 h at -80° C. Afterwards the samples were centrifuged at 4° C. for 30 min (13200 rpm; Centrifuge 5415R, Eppendorf), the supernatant was taken off, and the DNA pellets were dried at 37° C. The DNA samples were dissolved in ddH₂O.

After the third ligation, the DNA samples were gel extracted using the “QIAquick Gel Extraction Kit” (Qiagen) according to the manufacturer protocol.

In the following table the DNA sequences, which were involved in the ligation process, are depicted (See also FIG. 4):

TABLE 5

Sequences of the oligonucleotides I, IIₐ₋ᵢ, II′, III, III′, IV and IV′ (FIG. 4 )

| | | |
|---|---|---|
| DNA | length | sequence (5′-3′) |
| I | 37 mer | CAA ATC CGT TCA S AGG TCG GTG TGA ACG GAT TTG AGT C (SEO ID NO:4) |
| IIₐ₋ᵢ | 16 mer | IIₐ: CT^{∗}C TCT A^{∗}TC TA^{∗}T A^{∗}CC T (SEQ ID NO:30) |
| | | II_{b}: CT^{∗}C TTC A^{∗}A^{∗}T TCC A^{∗}CC T (SEQ ID NO:31) |
| | | II_{c}: CT^{∗}C TA^{∗}C A^{∗}CT TTA^{∗} A^{∗}CC T (SEQ NO:32) |
| | | II_{d}: CT^{∗}C TCA^{∗} A^{∗}TT A^{∗}CA^{∗} A^{∗}CC T (SEQ ID NO:33) |
| | | IIₑ: CT^{∗}C TA^{∗}C CTA^{∗} CTT A^{∗}CC T (SEQ ID NO:34) |
| | | II_{f}: CT^{∗}C TCT G^{∗}TC TG^{∗}T A^{∗}CC T (SEQ ID NO:35) |
| | | II_{g}: CT^{∗}C TCG^{∗} A^{∗}TT CG^{∗}C A^{∗}CC T (SEQ ID NO:36) |
| | | IIₕ: CT^{∗}C TCA^{∗} G^{∗}A^{∗}T TTC A^{∗}CC T (SEQ ID NO:37) |
| II′ | 24 mer | ATA CAG GTi aai iaa iAG AGG ACT (SEQ ID NO:17) |
| III | 12 mer | GTA TCA AGC AGG (SEQ ID NO: 18) |
| III′ | 12 mer | TAG GCC TGC TTG (SEQ ID NO: 19) |
| IV | 35 mer | CCT ACT CTC GTA TGA CCT CAA CTA CAT GGT CTA CA (SEQ ID NO: 20) |
| IV′ | 31 mer | TGT AGA CCA TGT AGT TGA GGT CAT ACG AGA G (SEQ ID NO:21) |

A* denotes 7-deaza adenosine; G* denotes 7-deaza-8-aza guanosine; i denotes inosine; a denotes abasic site; S denotes C₉ spacer; T* denotes T that serves as an attachment point to the linker/organic molecule; for validation purpose a benzyl substituent was attached via a triazole to a dU nucleotide by copper-mediated alkyne-azide cycloaddition of in situ formed benzyl azide to 5-ethynyl-dU. T* is here dU with a triazole-benzyl moiety in the 5-position.

TABLE 6

Functions of the partial sequences of oligonucleotides I, IIₐ₋ᵢ, II′, III, III′, IV and IV′ (FIG. 4 )

| | | | |
|---|---|---|---|
| partial sequence | function of partial sequence | length | sequence (5′-3′) |
| A | forward primer | 21 mer | AGG TCG GTG TGA ACG GAT TTG (SEO ID NO:9) |
| B | overhang for T4 DNA ligation | 4 mer | AGTC |
| S | spacer (Taq DNA polvmerase stopper) | - | C₉ spacer |
| B′ | overhang for T4 DNA ligation | 4 mer | GACT |
| Cₐ₋ᵢ | chemically modified DNA code (code 1) | 8 mer | Cₐ: CTA^{∗} TCT A^{∗}T |
| | | | C_{b}: TCA^{∗} A^{∗}TT CC |
| | | | C_{c}: A^{∗}CA^{∗} CTT TA^{∗} |
| | | | C_{d}: CA*A^{∗} TTA^{∗} CA^{∗} |
| | | | Cₑ: A^{∗}CC TA^{∗}C TT |
| | | | C_{f}: CTG^{∗} TCT G^{∗}T |
| | | | C_{g}: CG^{∗}A^{∗} TTC G^{∗}C |
| | | | Cₕ: CA^{∗} G^{∗}A^{∗}T TTC |
| C′ | universal sequence to chemically modified DNA code (code 1) | 8 mer | i aai iaa i |
| D | overhang for T4 DNA ligation | 4 mer | GTAT |
| D′ | overhang for T4 DNA ligation | 4 mer | TAGG |
| E | chemically un-modified DNA code (code 2) | 8 mer | CAA GCA GG |
| E′ | complementary sequence to chemically un-modified | 8 mer | GTT CGT CC |
| F | overhang for T4 DNA ligation | 4 mer | CCTA |
| F′ | overhang for T4 DNA ligation | 4 mer | TAGG |
| G | chemically un-modified DNA code (code 3) | 8 mer | CTC TCG TA |
| G′ | complementary sequence to chemically un-modified DNA code (code 3) | 8 mer | TACGAGAG |
| H | reverse primer | 23 mer | TGAC CTCA ACTA CATG GTCT ACA (SEO ID NO: 10) |
| H′ | complementary sequence to reverse primer | 23 mer | TGTA GACC ATGT AGTT GAGG TCA (SEO ID NO: 11) |

A* denotes 7-deaza adenosine; i denotes inosine; a denotes abasic site.

Agarose gel analysis of the ligation reaction of the hairpin DNA I with the chemically modified duplex DNA IIₐ₋ₑ/II′ (7-deaza adenosine was used) and native duplex DNA III/III′ with a marker panel and unligated DNA hairpin I as a reference showed successful and quantitative ligation of the three DNA molecules (FIG. 8).

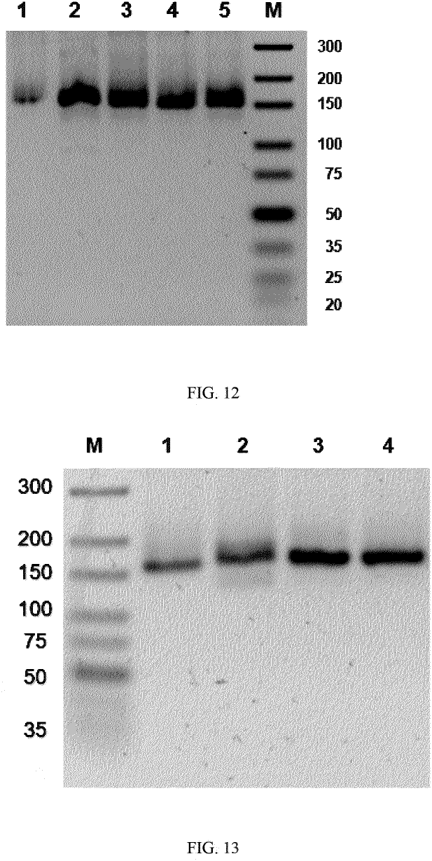

Agarose gel analysis of the ligation reaction of the chemically modified ligation product I-IIₐ₋ ₑIII/II′-III′ (7-deaza adenosine was used) with native duplex DNA IV/IV′ showed successful ligation of the two DNA molecules (FIG. 9).

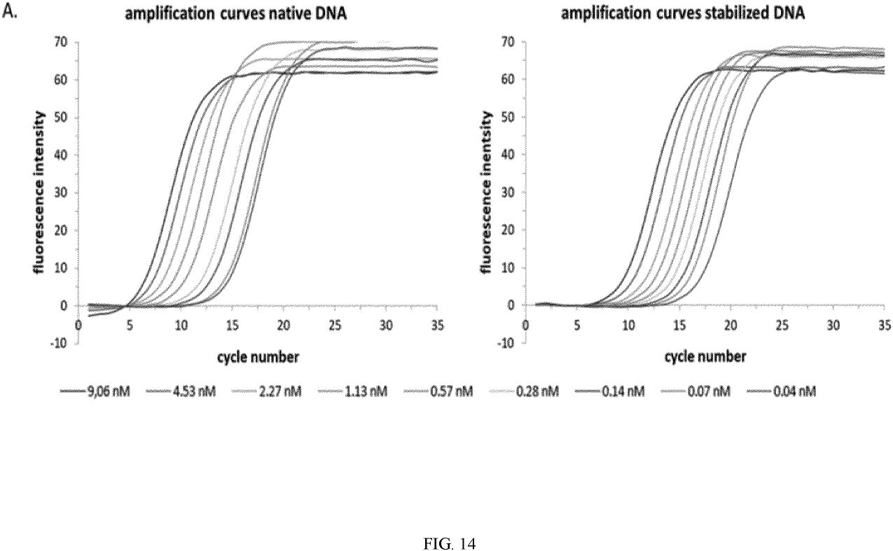

Agarose gel analysis of the ligation reaction of the hairpin DNA I with the chemically modified duplex DNA II_{f-i}/II′ (7-deaza adenosine and 7-deaza-8-aza guanosine was used) and native duplex DNA III/III′ with a marker panel and unligated DNA hairpin I as a reference showed successful and quantitative ligation of the three DNA molecules (FIG. 10).

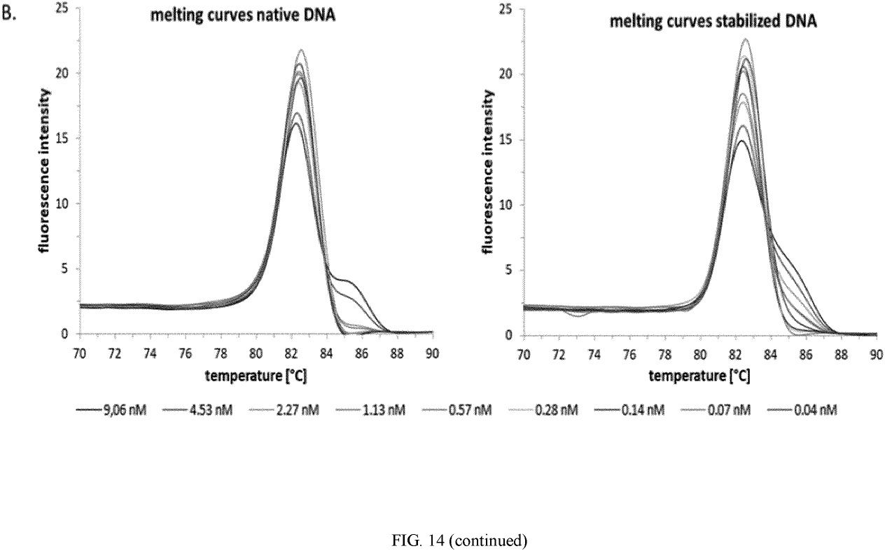

Agarose gel analysis of the ligation reaction of the chemically modified ligation product I-II_{f-} ᵢIII/II′-III′ (7-deaza adenosine and 7-deaza-8-aza guanosine was used) with native duplex DNA IV/IV′ showed successful ligation of the two DNA molecules (FIG. 11).

### Example 28: PCR Amplification of Chemically Modified Nucleic Acids

DNA obtained by enzymatic ligation of hairpin DNA I to chemically modified duplex DNA II/II′ and native duplex DNA III/III′ as well as IV/IV′ (FIGS. 8-11, Tables 5-6) were amplified by PCR (forward primer (54mer): 5′-TCG TCG GCA GCG TCA GAT GTG TAT AAG AGA CAG AGG TCG GTG TGA ACG GAT TTG-3′ (SEQ ID NO: 15), reverse primer (57mer): 5′-GTC TCG TGG GCT CGG AGA TGT GTA TAA GAG ACA GTG TAG ACC ATG TAG TTG AGG TCA-3′ (SEQ ID NO: 16).

For PCR amplification, 5 µL of gel extracted DNA, 5 U of Taq DNA polymerase (Thermo Fisher Scientific), 1x Taq Buffer (10 mM Tris-HCl, 50 mM KCl, 0.08% (v/v) Nonidet P40, pH = 8.8 at 25° C., Thermo Fisher Scientific), 3 mM MgCl2, 0.625 mM of each dNTP (dATP, dGTP, dTTP, and dCTP, corresponding to 2.5 mM of the mixture of dNTPs) and 1 µM of the reverse primer in a reaction volume of 39 µL. The PCR program started with pre-denaturation at 95° C. for 3 min, followed by denaturation for 30 s at 95° C., annealing for 30 s at 55° C., and elongation for 30 s at 72° C. After 10 cycles, 1 µM of the forward primer was added and additional 20 cycles were performed. After PCR, the time for elongation was prolonged to 5 min.

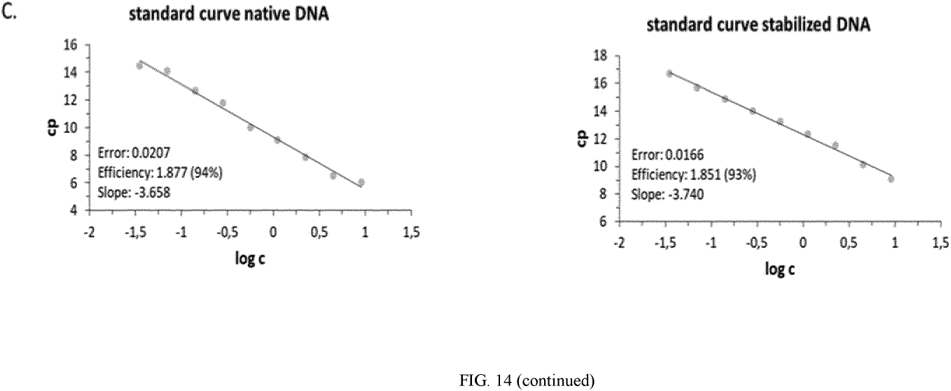

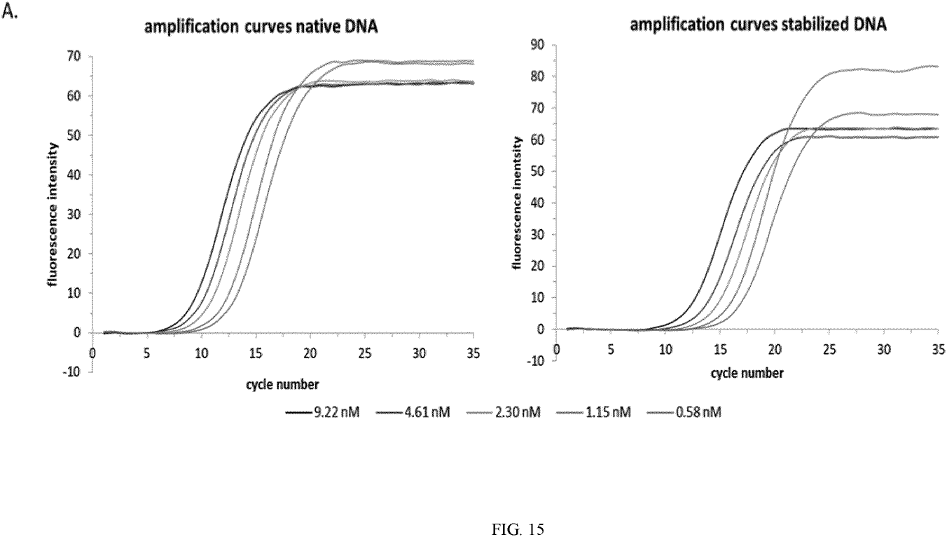

For analysis of DNA amplification reactions, agarose gel electrophoresis was performed. Using a 3% agarose gel, electrophoresis was carried out in TBE buffer (89 mM Tris-borate, 2 mM EDTA, pH = 8.3) at 150 V constant voltage for about 45 min. For staining of the DNA, Midori Green Direct (NIPPON Genetics) and as a reference, GeneRuler Ultra Low Range DNA Ladder (Thermo Fisher Scientific) was used. Imaging of the gels was performed using the Bio-Rad Gel Doc™ XR system. Results are shown in FIGS. 12-13.

### Example 29: Sequencing of Chemically Modified Nucleic Acids

DNA obtained by PCR amplification were purified using the QIAquick PCR Purification Kit (Qiagen) according to the protocol. Sequencing of the DNA was done by Microsynth Seqlab GmbH. Results showed that the chemically modified DNA was amplified and could be sequenced (data not shown).

### Example 30

For qPCR experiments the following were combined in PCR plate wells (GK480K-50, Kisker) in a total volume of 20 µL: DNA template (5 µL, ligation product 3), 200 nM forward primer (0.8 µL, 5 µM stock), 200 nM reverse primer (0.8 µL, 5 µM stock), SsoAdvanced universal SYBR® Green supermix (10 µL, Bio-Rad) and H2O (3.4 µL).

Forward primer: 5′-AGG TCG GTG TGA ACG GAT TTG AG-3′ (SEQ ID NO:22)

Reverse Primer: 5′-GTA GAC CAT GTA GTT GAG GTC A-3′ (SEQ ID NO:23)

For all qPCR experiments the following amplification method using the LightCycler® 480 II system from Roche was performed: hot start at 95° C. for 30 s, then 35 cycles of 95° C. for 15 s (denaturation), 60° C. for 30 s (annealing) and 72° C. for 30 s (elongation).

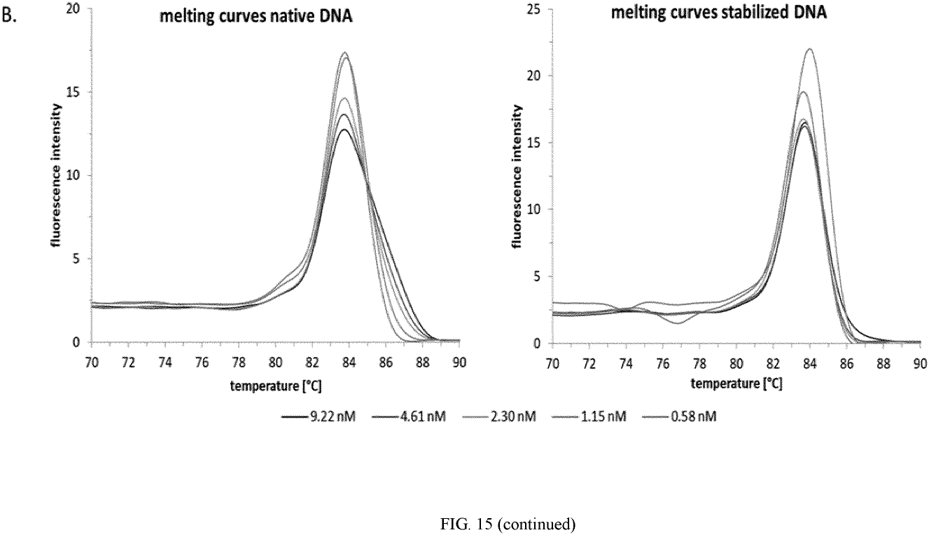

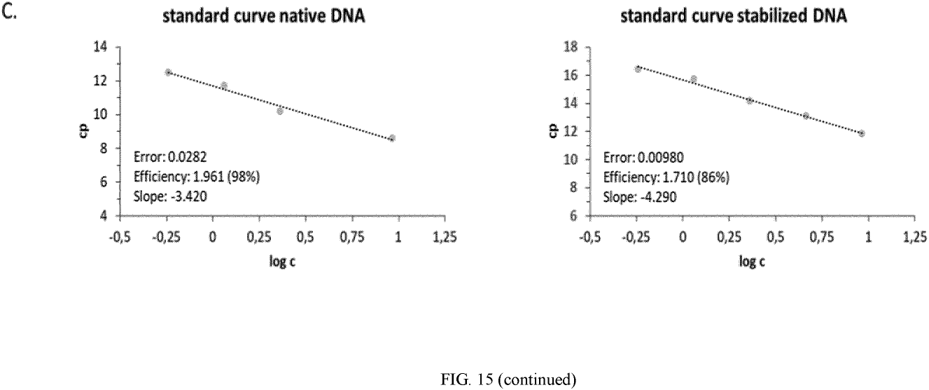

The specificity of the PCR amplification was analyzed by melting curve measurements. Analysis was done with the LightCycler® 480 - software version 1.5. Results are shown in FIGS. 14 and 15.

All documents cited herein, are hereby incorporated by reference in their entirety.

The non-limiting embodiments illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention. The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group. Further embodiments of the invention will become apparent from the following claims.

1. A compound library comprising a plurality of conjugate molecules, wherein each conjugate molecule comprises a small organic molecule covalently coupled to a nucleic acid moiety, wherein the nucleic acid moiety comprises or consists of a first nucleic acid selected from 7-deazapurines, 7-deaza-8-azapurines, and combinations thereof, and optionally modified pyrimidine nucleotides and/or unmodified pyrimidine nucleotides.

2. The compound library according to claim 1, wherein the first nucleic acid is selected from 7-deaza-8-azaadenosine, 7-deaza-8-azaguanosine, or combinations thereof.

3. The compound library according to claim 1, wherein the first nucleic acid comprises 7-aza-or 7-deaza-8-aza-modified inosine, 7-aza- or 7-deaza-8-aza-modified N⁶-methyladenosine, 7-aza- or 7-deaza-8-aza-modified xanthosine, or combinations thereof.

4. The compound library according to claim 1, wherein the plurality of conjugate molecules comprises at least ten conjugate molecules.

5. The compound library according to claim 1, wherein the nucleic acid moiety comprises at least 2 nucleotides.

6. The compound library according to claim 1, wherein the nucleic acid moiety comprises a sequence element of 3 to 18 nucleotides in length on its 5′ or 3′ end that consists exclusively of 7-deazapurines, 7-deaza-8-azapurines, and combinations thereof, and optionally modified pyrimidine nucleotides and/or unmodified pyrimidine nucleotides.

7. The compound library according to claim 1, wherein each conjugate has the structure 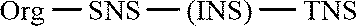
wherein “Org” represents the small organic molecule,
“SNS” represents a first nucleic acid identifier sequence that consists of 7-deazapurines and/or 7-deaza-8-azapurines, and, optionally, modified pyrimidine nucleotides and/or unmodified pyrimidine nucleotides,
“INS” represents an optional second nucleic acid identifier sequence that consists of 7-deazapurines and/or 7-deaza-8-azapurines, and, optionally, modified pyrimidine nucleotides and/or unmodified pyrimidine nucleotides, and
“TNS” represents a terminal nucleic acid identifier sequence that comprises unmodified purine residues,
wherein each of SNS and TNS is 3 to 18 nucleotides in length and INS is 3 to 50 nucleotides in length.

8. The compound library according to claim 1, wherein the nucleic acid moiety is RNA, DNA, or a hybrid thereof.

9. The compound library according to claim 1, wherein:
a) the conjugate molecules further comprise a linker between the nucleic acid moiety and the organic molecule;
b) the conjugate molecules differ from each other by comprising different nucleic acid moieties;
c) the organic molecule consists of 2 or more carbon atoms ; and/or
d) the organic molecule has a molecular weight of at most 900 daltons.

10. A method for synthesizing a compound library, wherein said library comprises a plurality of conjugate molecules, wherein said conjugates comprise a small organic molecule covalently coupled to a nucleic acid moiety, the synthesis of each conjugate molecule comprises:
reacting a first nucleic acid consisting of 7-deazapurines, 7-deaza-8-azapurines, and combinations thereof, and, optionally modified pyrimidine nucleotides and/or unmodified pyrimidine nucleotides, with an organic molecule under conditions to allow the conjugation of said molecules.

11. The method according to claim 10, wherein the 7-first nucleic acid is selected from 7-deaza-8-azaadenosine, 7-deaza-8-azaguanosine, or combinations thereof.

12. The method according to claim 10, wherein the first nucleic acid comprises 7-aza- or 7-deaza-8-aza-modified inosine, 7-aza- or 7-deaza-8-aza-modified N⁶-methyladenosine, 7-aza- or 7-deaza-8-aza-modified xanthosine, or combinations thereof.

13. The method according to claim 10, wherein reacting the nucleic acid with the organic molecule and subjecting the conjugate obtained to reaction conditions that allow modification of the small organic molecule comprise:
reacting the first nucleic acid with a first organic intermediate molecule under conditions to form a conjugate of the first nucleic acid and the first organic intermediate molecule; and
reacting the first organic intermediate molecule of the conjugate with a second organic intermediate molecule to form a conjugate molecule of the first nucleic acid and the reaction product of the first and second organic intermediate molecules.

14. The method according to claim 10, wherein the elongating the first nucleic acid moiety further comprises:
a) phosphorylating a 5′-terminal end of a sequence of the nucleic acid moiety; and
b) ligating a further nucleic acid sequence by a T4 ligase.

15. The method according to claim 10, wherein the method further comprises:
subjecting the conjugate to reaction conditions that allow modification of the small organic molecule; and
elongating the first nucleic acid moiety of the conjugate by adding a further nucleic acid sequence.

16. The method of claim 15, wherein the further nucleic acid sequence serves as an identifier nucleic acid sequence for the modified small organic molecule and the first nucleic acid sequence identifies the organic molecule.

17. The method of claim 15, wherein subjecting the conjugate to reaction conditions and elongating the first nucleic acid moiety are repeated at least once.

18. The method of claims 17, wherein the further nucleic acid sequence added in the first elongation is a second nucleic acid sequence and the further nucleic acid sequence added in the second or further elongation is a third or further nucleic acid sequence; wherein the second, third and further nucleic acid sequences are different from the fist nucleic acid sequence and different from each other.

19. The method of claim 10, wherein the further nucleic acid sequence added in the final elongation comprises unmodified purine nucleotides.

20. (canceled)

21. The method according to claim 10, wherein the nucleic acid moiety
a) consists of 2 to 20 nucleotides;
b) is RNA, DNA, or a mixture thereof;
c) is elongated in an additional reaction with modified and/or unmodified pyrimidine and/or purine nucleotides in the elongation step; and/or
d) combinations thereof.

22. (canceled)

23. (canceled)

24. (canceled)

25. (canceled)
